(19) United States
(12) Patent Application Publication
(10) Pub. No.: US 2021/0238249 A1
(43) Pub. Date: Aug. 5, 2021

(54) MODIFIED MITOCHONDRIA AND USE THEREOF

(71) Applicant: PAEAN BIOTECHNOLOGY INC., Daejeon (KR)

(72) Inventors: Kyuboem HAN, Daejeon (KR); Chun-Hyung KIM, Daejeon (KR); Yu Jin KIM, Daejeon (KR); Shin-Hye YU, Daejeon (KR); Nayoung KIM, Daejeon (KR); Mi Jin KIM, Daejeon (KR); Yong-Soo CHOI, Daejeon (KR); Seo Eun LEE, Daejeon (KR)

(21) Appl. No.: 17/049,301

(22) PCT Filed: Apr. 25, 2019
(86) PCT No.: PCT/KR2019/005020

§ 371 Date: Oct. 20, 2020

(30) Foreign Application Priority Data
Apr. 26, 2018 (KR) 10-2018-0048486

(51) Int. Cl. C07K 14/705; A61P 35/00; C07K 14/47; C12N 9/64; C12N 9/16; C07K 16/32; C07K 16/28; A61P 35/04

(52) U.S. Cl. CPC C07K 14/705; A61K 38/00; C07K 14/47; C07K 14/4746; C12N 9/6467; C12Y 304/21079; C12Y 301/03067; C12N 9/16; C07K 16/32; C07K 16/2854; C07K 16/2827; A61P 35/04; C07K 2319/30; C07K 2319/50; C07K 2319/95; A61P 35/00

(57) ABSTRACT

Mitochondria modified by a targeting protein, according to one embodiment of the present invention, can be effectively delivered to a target. In addition, when a protein of interest bound to the modified mitochondria is delivered into a cell, various activities can be exhibited. The modified mitochondria can effectively cause cancer tissue death, and thus can also be used as an anticancer agent. Furthermore, various activities are exhibited according to a protein of interest loaded on modified mitochondria, and thus the modified mitochondria can be applied in the treatment of various diseases. Additionally, a fusion protein comprising a protein of interest and a fusion protein comprising a targeting protein, according to one embodiment of the present invention, can be used in order to modify mitochondria. Moreover, mitochondria modified with the fusion proteins exhibits various effects in a target cell.

### TECHNICAL FIELD

The present invention provides a fusion protein capable of modifying mitochondria, mitochondria modified by the fusion protein, and a pharmaceutical composition comprising the same as an active ingredient.

### BACKGROUND ART

Mitochondria are cellular organelles of eukaryotic cells involved in the synthesis and regulation of adenosine triphosphate (ATP), an intracellular energy source. Mitochondria are associated with various metabolic pathways in vivo, for example, cell signaling, cell differentiation, cell death, as well as control of cell cycle and cell growth. Mitochondria have their own genomes and are organelles that play a central role in the energy metabolism of cells. Mitochondria produce energy through the electron transport and oxidative phosphorylation process, and play an important role in being involved in apoptosis signaling pathways.

It has been reported that a reduction in energy production due to a decrease in mitochondrial function causes various diseases. When the function of the electron transport chain reaction decreases according to the variation of the mitochondria genome and proteome, a reduction in ATP production, an excessive reactive oxygen production, a decrease in calcium regulation function and the like occur. In this case, a change in the membrane permeability of the mitochondria occurs, and the function of apoptosis may occur abnormally and lead to cancer and incurable diseases.

As such, human diseases that have been reported to result from mitochondrial dysfunction include mitochondria related genetic disease (Wallace D C 1999), diabetes mellitus (Maechler P 2001), heart disease (Sorescu D 2002), senile dementia such as Parkinson's disease or Alzheimer's disease (Lin M T 2006), and the occurrence of various cancers (Petros J A, 2005) and cancer metastasis (Ishikawa K, 2008) and the like have been reported. In addition, features commonly found in more than 200 types of various cancers consisted of impaired apoptosis function, increased inflammatory response, and increased abnormal metabolism. All of these processes are closely related to mitochondrial function, and the correlation between cancer and mitochondria is drawing attention.

On the other hand, it is known that normal cells produce 36 ATP per molecule of glucose through an electron transport system process, but cancer cells, unlike normal cells, produce 2 ATP per molecule of glucose through glycolysis under a sufficient oxygen condition (aerobic glycolysis). As such, it is known that cancer cells, unlike normal cells, use the inefficient glycolysis process in terms of energy in order to produce amino acids, lipids, nucleic acids and the like necessary for rapid cell proliferation. For this reason, it is known that cancer cells require less oxygen and produce a larger amount of lactic acid than normal cells.

Therefore, a change in the composition of the cancer microenvironment due to abnormal metabolism occurring in cancer cells, an inhibition of apoptosis caused by dysfunctional mitochondria, and an increase in inflammatory response, and abnormal metabolic reaction in cancer cells play a very important role in cancer proliferation. Thus, developing metabolism-related anticancer agents using these features may be a good way capable of solving the side effects and economic problems of conventional anticancer agents.

It is known that mitochondria enter into cells when the mitochondria present in the cells are isolated, and the cells are treated therewith in vitro, or the mitochondria are injected into the body. By using this phenomenon, it is possible that normal mitochondria isolated from cells are injected into the body to treat diseases caused by mitochondrial dysfunction, or in particular, to treat diseases by delivering effectively a specific protein into cells by using mitochondria as a carrier, but no reports have been made on this.

### Technical Problem

An object of the present invention is to provide an effective protein delivery system by showing that mitochondria can be used as a means to effectively deliver proteins capable of exhibiting various pharmacological effects into cells. In addition, an object of the present invention is to provide a recombinant protein for effectively delivering a drug, and to provide modified mitochondria that is produced using the same. In addition, an object of the present invention is to provide a pharmaceutical composition comprising the modified mitochondria as an active ingredient.

### Solution to Problem

In order to solve the above problems, there is provided modified mitochondria in which a foreign protein is bound to the outer membrane of the mitochondria. In addition, in order to prepare the modified mitochondria, there is provided a fusion protein comprising a mitochondrial outer membrane anchoring peptide and a desired pharmacological protein. In addition, there is provided a fusion protein comprising an antibody or a fragment thereof and a mitochondrial outer membrane anchoring peptide.

### Effect of the Invention

When the mitochondria to which the foreign protein is bound are administered to the human body, the foreign protein may be effectively delivered into the cell. In addition, the damaged function of the cell can be repaired by a pharmacologically active protein delivered into the cell. In addition, when the mitochondria to which the foreign protein comprising a pharmacologically active protein is bound are delivered into the cell, the pharmacologically active protein is dissociated from the mitochondria in the cell, and a useful role can be expected. In addition, the modified mitochondria comprising an antibody fragment may be effectively delivered to targeted cells. In particular, when a fragment of an antibody targeting a protein present on the surface of cancer tissue is bound to the surface of the mitochondria, the modified mitochondria may be effectively delivered into cancer cells. Therefore, the introduction of the modified mitochondria not only may restore the damaged electron transport system of the cells, but also may prevent or treat various diseases by the pharmacologically active protein bound to the modified mitochondria.

### BRIEF DESCRIPTION OF DRAWINGS

FIG. 1 is a schematic diagram of a method for preparing pTA-p53.

FIG. 2 is a schematic diagram of a method for preparing a pET15b-UB-p53 vector.

FIG. 3 shows the expression of a UB-p53 protein in E. coli.

FIG. 4 is a schematic diagram of a method for preparing a pET11C-TOM70-UB-p53 vector.

FIG. 5 shows the expression of a TOM70-UB-p53 protein in E. coli.

FIG. 6 is a schematic diagram of a method for preparing a pET11C-TOM70-(GGGGS)3-UB-p53 vector.

FIG. 7 shows the expression of a TOM70-(GGGGS)3-UB-p53 protein in E. coli.

FIG. 8 shows a method of preparing a pET11C-TOM70-(GGGGS)3-p53 vector.

FIG. 9 shows the expression of a TOM70-(GGGGS)3-p53 protein in E. coli.

FIG. 10 shows a method of preparing a pET15b-UB-p53-TOM7 vector.

FIG. 11 shows the expression of a UB-p53-TOM7 protein in E. coli.

FIG. 12 shows a method of preparing a pCMV-p53-myc/His vector.

FIG. 13 shows the expression of a p53-myc/His protein in transformed CHO.

FIG. 14 shows the results of purifying a TOM70-(GGGGS)3-p53 protein and then identifying the same.

FIG. 15 is a view showing a purified TOM70-(GGGGS)3-p53 protein.

FIG. 16 shows the results of purifying a TOM70-(GGGGS)3-UB-p53 protein and then identifying the same.

FIG. 17 is a view showing a purified TOM70-(GGGGS)3-UB-p53 protein.

FIG. 18 shows the results of purifying a UB-p53 protein and then identifying the same.

FIG. 19 is a view showing a purified UB-p53 protein.

FIG. 20 shows the results of purifying a UB-p53-TOM7 protein and then identifying the same.

FIG. 21 is a view showing a purified UB-p53-TOM7 protein.

FIG. 22 shows a method of preparing a pTA-GranzymeB vector.

FIG. 23 shows a method of preparing a pET11C-TOM70-(GGGGS)3-UB-GranzymeB vector.

FIG. 24 shows the expression of a TOM70-(GGGGS)3-UB-GranzymeB protein in E. coli.

FIG. 25 shows a method of preparing a pET15b-UB-GranzymeB-TOM7 vector.

FIG. 26 shows the expression of a UB-GranzymeB-TOM7 protein in E. coli.

FIG. 27 shows the results of purifying a TOM70-(GGGGS)3-UB-Granzyme B protein.

FIG. 28 is a view showing a purified TOM70-(GGGGS)3-UB-GranzymeB protein.

FIG. 29 shows a method of preparing a pTA-RKIP vector.

FIG. 30 shows a method of preparing a pET11C-TOM70-(GGGGS)3-UB-RKIP vector.

FIG. 31 shows the expression of a TOM70-(GGGGS)3-UB-RKIP protein in E. coli.

FIG. 32 shows the results of purifying a TOM70-(GGGGS)3-UB-RKIP protein.

FIG. 33 is a view showing a purified TOM70-(GGGGS)3-UB-RKIP protein.

FIG. 34 shows a method of preparing a pTA-PTEN vector.

FIG. 35 shows a method of preparing a pET11C-TOM70-(GGGGS)3-UB-PTEN vector.

FIG. 36 shows the expression of a TOM70-(GGGGS)3-UB-PTE protein in E. coli.

FIG. 37 shows the results of purifying a TOM70-(GGGGS)3-UB-PTEN protein.

FIG. 38 is a view showing a purified TOM70-(GGGGS)3-UB-PTEN protein.

FIG. 39 shows the results of purifying a UB-GFP-TOM7 protein and then identifying the same.

FIG. 40 is a view showing a purified UB-GFP-TOM7 protein.

FIG. 41 shows the results of purifying a TOM70-(GGGGS)3-UB-GFP protein and then identifying the same

FIG. 42 is a view showing a purified TOM70-(GGGGS)3-UB-GFP protein.

FIG. 43 shows a method of preparing a pET15b-UB-scFvHER2-TOM7 vector.

FIG. 44 shows the expression of a UB-scFvHER2-TOM7 protein in E. coli.

FIG. 45 shows a method of preparing a pCMV-scFvHER2-TOM7-myc/His vector.

FIG. 46 shows the expression of a scFvHER2-TOM7-myc/His protein in transformed CHO.

FIG. 47 shows the results of purifying a UB-ScFvHER2-TOM7 protein.

FIG. 48 is a view showing a purified UB-ScFvHER2-TOM7 protein.

FIG. 49 shows a method of preparing a pET15b-UB-scFvMEL-TOM7 vector.

FIG. 50 shows the expression of a UB-scFvMEL-TOM7 protein in E. coli.

FIG. 51 shows a method of preparing a pCMV-scFvMEL-TOM7-myc/His vector.

FIG. 52 shows the expression of a scFvMEL-TOM7-myc/His protein in transformed CHO.

FIG. 53 shows a method of preparing a pCMV-scFvPD-L1-TOM7-myc/His vector.

FIG. 54 shows the expression of a scFvPD-L1-TOM7-myc/His protein in transformed CHO.

FIG. 55 is a view confirming whether a fluorescent protein is bound to the outer membrane of the mitochondria. In this case, the mitochondria are stained with MitoTracker CMXRos to show red color, and TOM70-UB-GFP shows green color. The area where the two portions are overlapped shows yellow color. In this case, the magnification of 55a is 200-fold, and the magnification of 55b is 600-fold.

FIG. 56 shows the results of identifying the recombinant protein TOM70-(GGGGS)3-UB-p53 and UB-p53-TOM7 bound to the outer membrane of the foreign mitochondria using Western blot analysis.

FIG. 57 shows the results of observing the degree of intracellular injection according to the concentration of mitochondria using a fluorescence microscope after isolation of foreign mitochondria, and then injection of the mitochondria into cells.

FIG. 58 is a view confirming the influence of normal mitochondria on the proliferation of skin cancer cells.

FIG. 59 is a view confirming the influence of normal mitochondria on the inhibition of reactive oxygen species (ROS) production in skin cancer cells.

FIG. 60 is a view confirming the influence of normal mitochondria on drug resistance.

FIG. 61 is a view confirming the influence of normal mitochondria on the expression of an antioxidant gene in cells.

FIG. 62 is a view showing the influence of normal mitochondria on the expression of a gene involved in cancer cell metastasis.

FIG. 63 is a schematic diagram of a method for confirming loading of the recombinant protein p53 on the outer membrane of the foreign mitochondria and injection of the recombinant protein p53 into the cell.

FIG. 64 is a view confirming that the recombinant protein p53 is loaded on the outer membrane of the foreign mitochondria and that the p53 is injected into the cell. In this case, the magnification is 200-fold.

FIG. 65 is a view confirming that the recombinant protein p53 is loaded on the outer membrane of the foreign mitochondria and that the p53 is injected into the cell. In this case, the magnification is 600-fold.

FIG. 66 is a schematic diagram of a method for confirming the apoptosis ability of the modified mitochondria on which p53 injected into the cells is loaded, using a gastric cancer cell line.

FIG. 67a is a view confirming the apoptosis ability of the modified mitochondria on which p53 injected into gastric cancer cells is loaded, through a TUNEL assay. In this case, the magnification is 600-fold.

FIG. 67b is a view confirming the apoptosis ability of the modified mitochondria on which p53 injected into gastric cancer cells is loaded, through a fluorescence measurement.

FIG. 68 is a view confirming the effect of inhibiting cancer cell metastasis by the modified mitochondria loaded with RKIP in MDA-MB-231 cells.

FIG. 69 is a view confirming that a single chain variable fragment (ScFv) antibody for targeting cancer cells is expressed in cells.

FIG. 70 is a view confirming that a single chain variable fragment (ScFv) antibody for targeting cancer cells is expressed and bound to mitochondria present in the cell using an immunocytochemistry (ICC) experimental method. In this case, the magnification is 200-fold.

FIG. 71 is a view confirming that a single chain variable fragment (ScFv) antibody for targeting cancer cells is expressed and bound to mitochondria present in the cell using an immunocytochemistry (ICC) experimental method. In this case, the magnification is 600-fold.

FIG. 72 is a view comparing the effect of injecting the mitochondria to which a single chain variable fragment antibody for targeting cancer cells is bound into the gastric cancer cell line.

FIG. 73 is a schematic diagram of an animal experiment schedule using the modified mitochondria.

FIG. 74 is a photograph in which an increase in a tumor tissue is visually observed.

FIG. 75 is a view confirming the change in body weight of mice after administration of the mitochondria and the modified mitochondria.

FIG. 76 is a view confirming the tumor size after administration of the mitochondria and the modified mitochondria.

FIG. 77 is a view confirming that the modified mitochondria loaded with the TOM-UB-p53 protein is effective in inhibiting the proliferation of A431 cells.

FIG. 78 is a view confirming the function of the isolated mitochondria by ATP content.

FIG. 79 is a view confirming the function of the isolated mitochondria by membrane potential.

FIG. 80 is a view confirming the degree of damage of isolated mitochondria by measuring the mitochondrial ROS (mROS production)

FIG. 81a is a view showing the structure of the protein present in the outer membrane of the mitochondria and the amino acid sequence of the N terminal region of TOM70, TOM20 or OM45.

FIG. 81b is a view showing the amino acid sequence of the C terminal region of TOM5, TOM7, Fis1, VAMP1B, Cytb5, BCL-2 or BCL-X.

FIG. 82 is a view confirming whether the desired protein is dissociated according to the presence or absence of a linker between the outer membrane anchoring peptide and ubiquitin.

FIG. 83 is a view confirming that the desired protein bound to the modified mitochondria is separated off from the mitochondria in the cell.

### BEST MODE FOR CARRYING OUT THE INVENTION

Hereinafter, the present invention will be described in detail.

One aspect of the present invention provides modified mitochondria in which a foreign protein is bound to the outer membrane of the mitochondria.

The mitochondria may be obtained from mammals, and may be obtained from humans. Specifically, the mitochondria may be isolated from cells or tissues. For example, the mitochondria may be obtained from somatic cells, germ cells, or stem cells. In addition, the mitochondria may be normal mitochondria obtained from cells in which the biological activity of mitochondria is normal. In addition, the mitochondria may be cultured in vitro.

In addition, the mitochondria may be obtained from an autologous, allogenic, or xenogenic subject. Specifically, the autologous mitochondria refer to mitochondria obtained from tissues or cells of the same subject. In addition, the allogenic mitochondria refer to mitochondria obtained from a subject that belongs to the same species as the subject and has different genotypes for alleles. In addition, the xenogenic mitochondria refer to mitochondria obtained from a subject that belongs to the different species from the subject.

Specifically, the somatic cells may be muscle cells, hepatocytes, nerve cells, fibroblasts, epithelial cells, adipocytes, osteocytes, leukocytes, lymphocytes, platelets, or mucosal cells. In addition, the germ cells are cells that undergo meiosis and mitosis, and may be sperms or eggs. In addition, the stem cells may be any one selected from the group consisting of mesenchymal stem cells, adult stem cells, induced pluripotent stem cells, embryonic stem cells, bone marrow stem cells, neural stem cells, limbal stem cells, and tissue-derived stem cells. In this case, the mesenchymal stem cells may be any one selected from the group consisting of umbilical cord, umbilical cord blood, bone marrow, fat, muscle, nerve, skin, amniotic membrane, and placenta.

On the other hand, when the mitochondria are isolated from specific cells, the mitochondria can be isolated through various known methods, for example, using a specific buffer solution or using a potential difference and a magnetic field and the like.

As used herein, the term “foreign protein” refers to a protein that includes a desired protein capable of functioning inside and outside the cell. In this case, the foreign protein is a protein that does not exist in the mitochondria and may be a recombinant protein. Specifically, the foreign protein may comprise a mitochondria anchoring peptide and a desired protein. In addition, the foreign protein may be a recombinant fusion protein comprising a mitochondria anchoring peptide and a desired protein. In this case, the foreign protein may comprise a mitochondria anchoring peptide. Preferably, the mitochondria anchoring peptide may be a peptide that can be located on the mitochondrial outer membrane. Therefore, the foreign protein can be bound to the outer membrane of the mitochondria by a mitochondria anchoring peptide. The mitochondria anchoring peptide may be a peptide comprising an N terminal region or a C terminal region of a protein present in a mitochondrial membrane protein, and the N terminal region or the C terminal region of a protein present in the outer membrane of the mitochondria protein may be located on the outer membrane of the mitochondria. In this case, the anchoring peptide may further comprise a mitochondria signal sequence.

One embodiment of the protein present in a mitochondrial membrane protein may be any one selected from the group consisting of TOM20, TOM70, OM45, TOM5, TOM6, TOM7, TOM22, Fis1, Bcl-2, Bcl-x and VAMP1B. In particular, when the mitochondria anchoring peptide is derived from any one selected from the group consisting of TOM20, TOM70 and OM45, it may comprise the N terminal region of TOM20, TOM70 or OM45. One embodiment of the mitochondria anchoring peptide may be TOM70 derived from yeast represented by SEQ ID NO: 75, or TOM70 derived from human represented by SEQ ID NO: 76. Another embodiment may be TOM20 derived from yeast represented by SEQ ID NO: 77, or TOM20 derived from human represented by SEQ ID NO: 78. Another embodiment may be OM45 derived from yeast represented by SEQ ID NO: 79.

In addition, when the mitochondria anchoring peptide is derived from any one selected from the group consisting of TOM5, TOM6, TOM7, TOM22, Fis1, Bcl-2, Bcl-x and VAMP1B, it may comprise the C terminal region of any one selected from the group consisting of TOM5, TOM6, TOM7, TOM22, Fis1, Bcl-2, Bcl-x and VAMP1B. One embodiment of the mitochondria anchoring peptide may be TOM5 derived from yeast represented by SEQ ID NO: 80 or TOM5 derived from human represented by SEQ ID NO: 81. Another embodiment may be TOM7 derived from yeast represented by SEQ ID NO: 82, or TOM7 derived from human represented by SEQ ID NO: 83. Another embodiment may be TOM22 derived from yeast represented by SEQ ID NO: 84, or TOM22 derived from human represented by SEQ ID NO: 85. Another embodiment may be Fis1 derived from yeast represented by SEQ ID NO: 86, or Fis1 derived from human represented by SEQ ID NO: 87. Another embodiment may be Bcl-2 alpha derived from human represented by SEQ ID NO: 88. Another embodiment may be VAMP1 derived from yeast represented by SEQ ID NO: 89, or VAMP1 derived from human represented by SEQ ID NO: 90.

In this case, a desired protein capable of functioning inside and outside the cell included in the foreign protein may be any one selected from the group consisting of an active protein exhibiting an activity in a cell, a protein present in a cell, and a protein having the ability to bind to a ligand or receptor present in a cell membrane.

An embodiment of the active protein or the protein present in a cell may be any one selected from the group consisting of p53, Granzyme B, Bax, Bak, PDCD5, E2F, AP-1(Jun/Fos), EGR-1, Retinoblastoma(RB), phosphatase and tensin homolog(PTEN), E-cadherin, Neurofibromin-2(NF-2), poly[ADP-ribose] synthase 1(PARP-1), BRCA-1, BRCA-2, Adenomatous polyposis coli(APC), Tumor necrosis factor receptor-associated factor(TRAF), RAF kinase inhibitory protein(RKIP), p16, KLF-10, LKB1, LHX6, C-RASSF, DKK-3PD1, Oct3/4, Sox2, Klf4, and c-Myc. When the desired protein is selected from the above group, the desired protein may be bound to an anchoring peptide comprising the N terminal region of TOM20, TOM70 or OM45.

Such fusion protein may be bound in the following order:

N terminal-anchoring peptide comprising the N terminal region of TOM20, TOM70 or OM45-desired protein-C terminal.

In addition, the foreign protein may further comprise an amino acid sequence recognized by a proteolytic enzyme in eukaryotic cells, or ubiquitin or a fragment thereof between a mitochondria anchoring peptide and a desired protein. The proteolytic enzyme in eukaryotic cells refers to an enzyme that degrades a protein present in eukaryotic cells. In this case, because a foreign protein comprises an amino acid sequence recognized by the enzyme that degrades the protein, the foreign protein bound to the mitochondrial outer membrane may be isolated into an anchoring peptide and a desired protein in a cell.

In this case, the ubiquitin fragment may comprise the C terminal Gly-Gly of an amino acid sequence of SEQ ID NO: 71, and may comprise 3 to 75 amino acids consecutive from the C terminal. In addition, the foreign protein may further comprise a linker between a desired protein and ubiquitin or a fragment thereof. In this case, the linker may be composed of 1 to 150 amino acids, or be composed of 10 to 100 amino acids, or be composed of 20 to 50 amino acids, but is not limited thereto. The linker may be composed of amino acids that are appropriately selected from 20 amino acids, preferably be composed of glycine and/or serine. One embodiment of the linker may be composed of 5 to 50 amino acids consisting of glycine and serine. One embodiment of the linker may be (G4S)n, in which n is an integer of 1 to 10, and n may be 1, 2, 3, 4, 5, 6, 7, 8, 9, or 10.

In addition, the protein having the ability to bind to a ligand or receptor present in a cell membrane may be a ligand or receptor present on the surface of a tumor cell. In this case, the ligand or receptor present on the surface of a tumor cell may be, but is not limited to, any one selected from the group consisting of CD19, CD20, melanoma antigen E(MAGE), NY-ESO-1, carcinoembryonic antigen(CEA), mucin 1 cell surface associated(MUC-1), prostatic acid phosphatase(PAP), prostate specific antigen(PSA), survivin, tyrosine related protein 1(tyrp1), tyrosine related protein 1(tyrp2), Brachyury, Mesothelin, Epidermal growth factor receptor(EGFR), human epidermal growth factor receptor 2(HER-2), ERBB2, Wilms tumor protein(WT1), FAP, EpCAM, PD-L1, ACPP, CPT1A, IFNG, CD274, FOLR1, EPCAM, ICAM2, NCAM1, LRRC4, UNC5H2 LILRB2, CEACAM, Nectin-3 and a combination thereof.

In addition, the protein having the ability to bind to a ligand or receptor present in a cell membrane may be an antibody or a fragment thereof that binds to any one selected from the above group. In particular, a fragment of an antibody refers to a fragment having the same complementarity determining region (CDR) as that of the antibody. Specifically, it may be Fab, scFv, F (ab′)2 or a combination thereof.

In this case, the desired protein may be bound to an anchoring peptide comprising an C terminal region of any one selected from the group consisting of TOM5, TOM6, TOM7, TOM22, Fis1, Bcl-2, Bcl-x and VAMP1B, and the foreign protein may be bound in the following order: N terminal-desired protein-anchoring peptide comprising a C terminal region of any one selected from the group consisting of TOM5, TOM6, TOM7, TOM22, Fis1, Bcl-2, Bcl-x and VAMP1B-C terminal.

In addition, the foreign protein may further comprise a linker between a desired protein and a C terminal region of any one selected from the group consisting of TOM5, TOM6, TOM7, TOM22, Fis1, Bcl-2, Bcl-x and VAMP1B. In this case, a linker is as described above. In this case, a desired protein, an active protein, a protein present in a cell, and a protein having the ability to bind to a ligand or receptor present in a cell membrane and the like are as described above.

In one embodiment of the desired protein, an antibody or a fragment thereof targeting a specific cell may be in a form bound to the anchoring peptide comprising the C terminal region of any one selected from the group consisting of TOM5, TOM6, TOM7, TOM22, Fis1, Bcl-2, Bcl-x and VAMP1B. The modified mitochondria to which the desired protein is bound can be easily introduced into a specific target, so that the mitochondria can be efficiently entered into a specific cell.

One embodiment of the modified mitochondria may be in a form to which one or more desired proteins are bound. Specifically, it may be in a form to which a desired protein comprising p53 and a desired protein comprising anti-HER-2 antibody or a fragment thereof are bound. Such modified mitochondria may effectively deliver the mitochondria into cancer cells expressing HER-2. In addition, cancer cells may be effectively killed by p53 bound to the modified mitochondria.

Depending on the purpose of the modified mitochondria, a desired protein comprising one or more active proteins may be constructed and be allowed to be bound to the mitochondria. In addition, a desired protein targeting a cell may be constructed in various ways depending on the targeted cell.

In another aspect of the present invention, there is provided a pharmaceutical composition comprising the above described modified mitochondria as an active ingredient. In this case, use of the pharmaceutical composition may be for the prevention or treatment of cancer. In this case, the cancer may be any one selected from the group consisting of gastric cancer, liver cancer, lung cancer, colorectal cancer, breast cancer, prostate cancer, ovarian cancer, pancreatic cancer, cervical cancer, thyroid cancer, larynx cancer, acute myeloid leukemia, brain tumor, neuroblastoma, retinoblastoma, head and neck cancer, salivary gland cancer and lymphoma.

Specifically, when the active protein kills tumor cells, like p53, or when a protein that inhibits the proliferation is bound to the mitochondria, the modified mitochondria to which p53 is bound may be used as an anticancer agent. In addition, when a protein such as RKIP capable of inhibiting metastasis of cancer cells is bound to the mitochondria, the modified mitochondria to which RKIP is bound may be used as an inhibitor of tumor metastasis. When any one selected from the group consisting of Granzyme B, Bax, Bak, PDCD5, E2F, AP-1(Jun/Fos), EGR-1, Retinoblastoma(RB), phosphatase and tensin homolog(PTEN), E-cadherin, Neurofibromin-2(NF-2), poly [ADP-ribose] synthase 1(PARP-1), BRCA-1, BRCA-2, Adenomatous polyposis coli(APC), Tumor necrosis factor receptor-associated factor(TRAF), p16, KLF-10, LKB1, LHX6, C-RASSF, DKK-3PD1 and a combination thereof, which are proteins that inhibit the proliferation of cancer cells, or control the phosphorylation reaction in cancer cells, or inhibit the metastasis of cancer cells, is bound to the mitochondria, the modified mitochondria to which the active protein is bound may be used as an anticancer agent.

In addition, for the pharmaceutical composition, the mitochondria may be included at a concentration of 0.1 μg/mL to 500 μg/mL, 0.2 μg/mL to 450 μg/mL, or 0.5 μg/mL to 400 μg/mL, but is not limited thereto. The inclusion of the mitochondria in the above range may facilitate the dose adjustment of mitochondria upon administration and may enhance the degree of improvement of the symptoms of a disease of a patient. In this case, the dose of mitochondria may be determined through the quantification of mitochondria by quantifying the membrane protein of the isolated mitochondria. Specifically, the isolated mitochondria may be quantified through the Bradford protein assay (a paper written by James D. McCully (J Vis Exp. 2014; (91): 51682.).

In addition, for the pharmaceutical composition, an active protein binding to mitochondria may be included at a concentration of 0.1 μg/mL to 500 μg/mL, 0.2 μg/mL to 450 μg/mL, or 0.5 μg/mL to 400 μg/mL, but is not limited thereto. The inclusion of the active protein in the above range may facilitate the dose adjustment of an active protein upon administration and may enhance the degree of improvement of the symptoms of a disease of a patient.

In addition, for the pharmaceutical composition, a targeting protein capable of delivering mitochondria to a specific cell may be included at a concentration of 0.1 μg/mL to 500 μg/mL, 0.2 μg/mL to 450 μg/mL or 0.5 μg/mL to 400 μg/mL, but is not limited thereto. The inclusion of the targeting protein in the above range may facilitate the dose adjustment of a targeting protein upon administration and may enhance the degree of improvement of the symptoms of a disease of a patient.

In particular, the pharmaceutical composition according to the present invention may be administered with mitochondria in an amount of, but not limited thereto, 0.01-5 mg/kg, 0.1-4 mg/kg, or 0.25-2.5 mg/kg per one time on the basis of the body weight of an individual to be administered. That is, it is most preferable in terms of the cell activity to administer the pharmaceutical composition such that the amount of the modified mitochondria falls within the above range on the basis of the body weight of an individual having cancer tissues. In addition, the pharmaceutical composition may be administered 1-10 times, 3-8 times, or 5-6 times, and preferably 5 times. In this case, the administration interval may be 1-7 days, or 2-5 days, and preferably 3 days.

In addition, the pharmaceutical composition according to the present invention may be administered to a human or other mammal that is susceptible to cancer or suffering from cancer. In addition, the pharmaceutical composition may be an injectable preparation that may be intravenously administered or an injectable preparation that may be topically administered, and may be preferably a preparation for injections.

Therefore, the pharmaceutical composition according to the present invention may be prepared as a physically or chemically highly stable injectable preparation by adjusting the pH of the composition by means of a buffer solution such as an acid aqueous solution or phosphate which may be used in an injectable preparation, in order to ensure the stability of the product during distribution of injectable preparations.

Specifically, the pharmaceutical composition of the present invention may contain water for injection. The water for injection is distilled water prepared for dissolving a solid injectable preparation or diluting a water-soluble injectable preparation, and may be glucose injection, xylitol injection, D-mannitol injection, fructose injection, saline, dextran 40 injection, dextran 70 injection, amino acid injection, Ringer's solution, lactic acid-Ringer's solution, phosphate buffer solution having a pH of 3.5 to 7.5, sodium dihydrogen phosphate-citrate buffer solution or the like.

In addition, the pharmaceutical composition of the present invention may include a stabilizer or a dissolution aid. For example, the stabilizer may be sodium pyrosulfite or ethylenediaminetetraacetic acid, and the dissolution aid may be hydrochloric acid, acetic acid, sodium hydroxide, sodium hydrogen carbonate, sodium carbonate or potassium hydroxide.

In addition, the present invention may provide a method for preventing or treating cancer including administering the above-mentioned pharmaceutical composition to an individual. Here, the individual may be a mammal, and preferably a human.

One aspect of the present invention provides a method for preparing the modified mitochondria, comprising a step of mixing the isolated mitochondria with a desired protein comprising an active protein and/or a desired protein comprising a target targeting protein.

In this case, the desired protein and the mitochondria may be mixed in an appropriate ratio. For example, the desired protein:mitochondria may be mixed in a ratio of 1:100 to 100:1 based on a weight ratio. Specifically, they may be mixed in a ratio of 1:10, 1:5, 1:4, 1:3, 1:2 or 1:1. In addition, the ratio may be 10:1, 5:1, 4:1, 3:1 or 2:1.

In another aspect of the present invention, there is provided a method for preparing the modified mitochondria from transformed cells by injecting a polynucleotide encoding the above described desired protein into eukaryotic cells. Specifically, there is provided a method for preparing the above described fusion protein, comprising a step of transforming the above described polynucleotide into prokaryotic cells or eukaryotic cells without a ubiquitin degrading enzyme or a proteolytic enzyme in eukaryotic cells; and a step of obtaining a fusion protein. This preparation method is suitable when the desired protein does not comprise an amino acid sequence recognized by a proteolytic enzyme in eukaryotic cells or ubiquitin or a fragment thereof.

In another aspect of the present invention, a desired protein may be prepared using a prokaryotic cell or a prokaryotic cell extract. In addition, there is provided a method for preparing the modified mitochondria using eukaryotic cells without a ubiquitin degrading enzyme or a proteolytic enzyme, or a eukaryotic cell extract.

In another aspect of the present invention, there is provided use of the mitochondria as a means of delivery of a foreign protein. Specifically, the modified mitochondria may be used as a means of intracellular and extracellular delivery of a foreign protein comprising a desired protein capable of functioning inside and outside the cell. The mitochondria may be effectively introduced into cells, and in this case, a foreign protein desired to be delivered to cells may be effectively delivered into cells. In this case, the mitochondria may be used as an effective protein delivery system. The desired protein is as described above.

Another aspect of the present invention provides a fusion protein comprising a mitochondrial outer membrane anchoring peptide and a desired protein. In this case, the desired protein is as described above.

As used herein, the term “mitochondrial outer membrane anchoring peptide” may be the N terminal or C terminal of a protein present in the outer membrane of the mitochondria. The mitochondrial outer membrane anchoring peptide may have an amino acid sequence that is specifically located in the outer membrane of the mitochondria. In this case, the mitochondrial outer membrane anchoring peptide allows the fusion protein disclosed in the present invention to be attached to the outer membrane of the mitochondria. In this case, the mitochondrial outer membrane anchoring peptide may be used in the same sense as the mitochondrial outer membrane targeting peptide.

In addition, the mitochondrial outer membrane anchoring peptide prevents the fusion protein disclosed in the present invention from entering the inside of the mitochondria. The TOM (translocase of the outer membrane) complex present in the mitochondrial outer membrane has a mitochondria target sequence and a single outer membrane anchoring domain at the amino terminus, and most of the carboxy terminus may have a structure that is exposed to the cytoplasm (FIG. 81a). The TOM (translocase of the outer membrane) complex present in the mitochondrial outer membrane has a mitochondria target sequence and a single outer membrane anchoring domain at the carboxyl terminus, and most of the amino terminus may also have a structure that is exposed to the cytoplasm (FIG. 81b). In addition, the protein present in the outer membrane of the mitochondria may be selected from proteins present in the mitochondria that are present in a eukaryotic cell. For example, it may be selected from proteins present in the mitochondrial outer membrane that are present in yeast, animal cells, or human cells.

In this case, an embodiment of the protein present in the mitochondrial outer membrane may be any one protein selected from the group consisting of TOM20, TOM70, OM45, TOM5, TOM6, TOM7, TOM22, Fis1, Bcl-2, Bcl-x and VAMP1B, or a fragment thereof. In this case, the mitochondrial outer membrane anchoring peptide may be a fragment of any one protein selected from the group consisting of TOM20, TOM70, OM45, TOM5, TOM6, TOM7, TOM22, Fis1, Bcl-2, Bcl-x and VAMP1B. In this case, the outer membrane anchoring peptide may be a C terminal or N terminal polypeptide of TOM20, TOM70, OM45, TOM5, TOM6, TOM7, TOM22, Fis1, Bcl-2, Bcl-x and VAMP1B located in the mitochondrial outer membrane.

In particular, when the mitochondrial outer membrane anchoring peptide is fused to the N terminal of the desired protein, the mitochondrial outer membrane anchoring peptide may comprise a terminal sequence of a protein selected from the group consisting of TOM20, TOM70, and OM45. Preferably, it may be an N terminal sequence of a protein selected from the group consisting of TOM20, TOM70, and OM45. An embodiment of the mitochondrial outer membrane anchoring peptide is as described above.

In addition, when the mitochondrial outer membrane anchoring peptide is fused to the C terminal of the desired protein, the outer membrane targeting protein may comprise a terminal sequence of a protein selected from the group consisting of TOM5, TOM6, TOM7, TOM22, Fis1, Bcl-2, Bcl-X, and VAMP1B. Preferably, it may be a C terminal sequence of a protein selected from the group consisting of TOM5, TOM6, TOM7, TOM22, Fis1, Bcl-2, Bcl-X, and VAMP1B. An embodiment of the mitochondrial outer membrane anchoring peptide is as described above.

As used herein, the term “active protein” may be a protein exhibiting physiological activity. One embodiment of such an active protein may be a protein having decreased function or a modified protein present in damaged cancer cells. One embodiment of the active protein may be a protein that enhances the activity of cells. An embodiment of such an active protein is as described above.

The fusion protein may be a protein to which a mitochondrial outer membrane targeting protein and a desired protein are bound from the N terminal to the C terminal. In this case, it may further comprise ubiquitin or a fragment thereof having a ubiquitin protease specific cleavage site (Glycin-Glycin) between the mitochondrial outer membrane targeting protein and the desired protein. In this case, in order to facilitate cleavage by the ubiquitin protease, it may further comprise a linker containing hydrophilic and polar amino acids, serine, glycine and threonine, between the mitochondrial outer membrane targeting protein and the ubiquitin protein.

As used herein, the term “ubiquitin” refers to a protein that participates in the proteolytic process, also referred to as UB. One embodiment of ubiquitin may be ubiquitin present in the human body or ubiquitin present in yeast. Ubiquitin present in the human body is composed of 76 amino acids. In this case, ubiquitin may be used in a mature form. As used herein, the term “mature form” may refer to a protein in a form from which a signal peptide is removed.

In addition, an enzyme referred to as ubiquitin protease or UBP (ubiquitin-specific protease) is naturally present in eukaryotic cells and may induce the natural dissociation of a desired protein by cleaving the C terminal amino acid glycine-glycine site of ubiquitin in a cell.

In this case, the fragment of ubiquitin may comprise the Gly-Gly amino acid of the C terminal of ubiquitin, and may comprise 3 to 75 amino acids consecutive from the C terminal. Specifically, an embodiment of the fragment of ubiquitin may be Arg-Gly-Gly, Leu-Arg-Gly-Gly, Arg-Leu-Arg-Gly-Gly, or Leu-Arg-Leu-Arg-Gly-Gly. In addition, the fragment of ubiquitin may have an amino acid sequence of SEQ ID NO: 71.

The fusion protein comprising the mitochondrial outer membrane targeting protein and the desired protein may be referred to as a fusion protein that modifies the mitochondria activity. Such fusion protein may have any one of the following structures:

<Structural Formula 1>

N terminal-mitochondrial outer membrane anchoring peptide-desired protein-C terminal

<Structural Formula 2>

N terminal-mitochondrial outer membrane anchoring peptide-ubiquitin or fragment thereof-desired protein-C terminal

<Structural Formula 3>

N terminal-mitochondrial outer membrane targeting peptide-linker 1-ubiquitin or fragment thereof-desired protein-C terminal

<Structural Formula 4>

N terminal-mitochondrial outer membrane anchoring peptide-ubiquitin or fragment thereof-linker 2-desired protein-C terminal

<Structural Formula 5>

N terminal-mitochondrial outer membrane anchoring peptide-linker 1-ubiquitin or fragment thereof-linker 2-desired protein-C terminal In the above Structural Formulae 1 to 5, the outer membrane anchoring peptide may be a terminal sequence of a protein selected from the group consisting of TOM20, TOM70 and OM45, and the desired protein may be any one selected from the group consisting of p53, Granzyme B, Bax, Bak, PDCD5, E2F, AP-1(Jun/Fos), EGR-1, Retinoblastoma(RB), phosphatase and tensin homolog(PTEN), E-cadherin, Neurofibromin-2(NF-2), poly[ADP-ribose] synthase 1 (PARP-1), BRCA-1, BRCA-2, Adenomatous polyposis coli(APC), Tumor necrosis factor receptor-associated factor(TRAF), RAF kinase inhibitory protein(RKIP), p16, KLF-10, LKB1, LHX6, C-RASSF and DKK-3PD1.

In this case, the linker 1 or 2 may be a polypeptide composed of 1 to 100, 1 to 80, 1 to 50, or 1 to 30 amino acids, respectively, and may be preferably a polypeptide composed of 1 to 30 amino acids that consist of serine, glycine or threonine alone or in combination. In addition, the linker 1 or 2 may be a polypeptide composed of 5 to 15 amino acids, respectively, and may be preferably a polypeptide composed of 5 to 15 amino acids that consist of serine, glycine or threonine alone or in combination. One embodiment of the linker may be (GGGGS)3 (SEQ ID NO: 70).

<Structural Formula 6>

N terminal-desired protein-mitochondrial outer membrane anchoring peptide-C terminal

<Structural Formula 7>

N terminal-desired protein-ubiquitin or a fragment thereof-mitochondrial outer membrane anchoring peptide-C terminal

<Structural Formula 8>

N terminal-desired protein-linker 1-ubiquitin or a fragment thereof-mitochondrial outer membrane anchoring peptide-C terminal

<Structural Formula 9>

N terminal-desired protein-ubiquitin or a fragment thereof-linker 2-mitochondrial outer membrane anchoring peptide-C terminal

<Structural Formula 10>

N terminal-desired protein-linker 1-ubiquitin or fragment thereof-linker 2-mitochondrial outer membrane targeting peptide-C terminal

In the above Structural Formulae 6 to 10, the outer membrane anchoring peptide may be a terminal sequence of a protein selected from the group consisting of TOM5, TOME, TOM7, TOM22, Fis1, Bcl-2, Bcl-X, and VAMP1B, and the desired protein may be any one selected from the group consisting of p53, Granzyme B, Bax, Bak, PDCD5, E2F, AP-1(Jun/Fos), EGR-1, Retinoblastoma(RB), phosphatase and tensin homolog(PTEN), E-cadherin, Neurofibromin-2(NF-2), poly [ADP-ribose] synthase 1(PARP-1), BRCA-1, BRCA-2, Adenomatous polyposis coli(APC), Tumor necrosis factor receptor-associated factor(TRAF), RAF kinase inhibitory protein(RKIP), p16, KLF-10, LKB1, LHX6, C-RASSF, DKK-3PD1, Oct3/4, Sox2, Klf4, and c-Myc. In this case, the linker 1 or 2 is as described above.

One aspect of the present invention provides a polynucleotide encoding a fusion protein comprising a mitochondrial outer membrane anchoring peptide and a desired protein.

In addition, one aspect of the present invention provides a vector loaded with the polynucleotide encoding a fusion protein comprising a desired protein.

In addition, one aspect of the present invention provides a host cell in which a vector loaded with a polynucleotide encoding a fusion protein comprising the desired protein is introduced.

One aspect of the present invention provides a fusion protein comprising a target targeting protein and a mitochondrial outer membrane targeting protein.

In this case, the target targeting protein and the mitochondrial outer membrane anchoring peptide may be bound from the N terminal to the C terminal. Here, the mitochondrial outer membrane anchoring peptide may be any one selected from the group consisting of TOM20, TOM70, OM45, TOM5, TOME, TOM7, TOM22, Fis1, Bcl-2, Bcl-x and VAMP1B.

As used herein, the term “target” refers to a place where the modified mitochondria should be delivered. One embodiment of the target may be a cancer cell. Specifically, one embodiment of the target may be a biomarker present on the surface of cancer cells. Specifically, the target may be a tumor-associated antigen (TAA). In this case, the tumor-associated antigen may be any one selected from the group consisting of CD19, CD20, melanoma antigen E(MAGE), NY-ESO-1, carcinoembryonic antigen(CEA), mucin 1 cell surface associated(MUC-1), prostatic acid phosphatase(PAP), prostate specific antigen(PSA), survivin, tyrosine related protein 1(tyrp1), tyrosine related protein 1(tyrp2), Brachyury, Mesothelin, Epidermal growth factor receptor(EGFR), human epidermal growth factor receptor 2(HER-2), ERBB2, Wilms tumor protein(WT1), FAP, EpCAM, PD-L1, ACPP, CPT1A, IFNG, CD274, FOLR1, EPCAM, ICAM2, NCAM1, LRRC4, UNC5H2 LILRB2, CEACAM, Nectin-3 and a combination thereof.

As used herein, the term “target targeting protein” may be a protein sequence capable of binding to the above described target. In this case, one embodiment of the target targeting protein may be a protein that binds to a biomarker present on the surface of cancer cells. In this case, an embodiment of the biomarker present on the surface of cancer cells may be, but is not limited to, ICAM2, NCAM1, LRRC4, UNC5H2 LILRB2, CEACAM, or Nectin-3. In this case, the target targeting protein may be included in the above described foreign protein.

One embodiment of the target targeting protein may be an antibody or a fragment thereof. In particular, it may be an antibody or a fragment thereof that specifically binds to the tumor-associated antigen. In addition, the fragment of the antibody may be any one selected from the group consisting of Fab, Fab′, scFv and F(ab)2.

One embodiment of the target targeting protein may be scFvHER capable of binding to an epidermal growth factor receptor. Another embodiment may be scFvMEL capable of targeting melanoma. Another embodiment may be scFvPD-L1 capable of binding to PD-L1 overexpressed on the surface of cancer cells. Another embodiment may be PD-1 capable of binding to PDL-1 overexpressed on the surface of cancer cells.

One aspect of the present invention may further comprise ubiquitin or a fragment thereof between the target targeting protein and the mitochondrial outer membrane targeting protein. The fusion protein comprising the mitochondria target targeting protein and the desired protein may be referred to as a fusion protein that modifies the mitochondria activity. Such fusion protein may have any one of the following structures:

<Structural Formula 11>

N terminal-target targeting protein-mitochondrial outer membrane anchoring peptide-C terminal

<Structural Formula 12>

N terminal-target targeting protein-ubiquitin or a fragment thereof-mitochondrial outer membrane anchoring peptide-C terminal

<Structural Formula 13>

N terminal-target targeting protein-linker 1-ubiquitin or a fragment thereof-mitochondrial outer membrane anchoring peptide-C terminal

<Structural Formula 14>

N terminal-target targeting protein-ubiquitin or fragment thereof-linker 2-mitochondrial outer membrane anchoring peptide-C terminal

<Structural Formula 15>

N terminal-target targeting protein-linker 1-ubiquitin or fragment thereof-linker 2-mitochondrial outer membrane anchoring peptide-C terminal

In the above Structural Formulae 11 to 15, the outer membrane anchoring peptide may be a terminal sequence of a protein selected from the group consisting of TOM5, TOME, TOM7, TOM22, Fis1, Bcl-2, Bcl-X, and VAMP1B, and the target targeting protein may be any one selected from the group consisting of tumor associated antigens, CD19, CD20, melanoma antigen E(MAGE), NY-ESO-1, carcinoembryonic antigen(CEA), mucin 1 cell surface associated(MUC-1), prostatic acid phosphatase(PAP), prostate specific antigen(PSA), survivin, tyrosine related protein 1(tyrp1), tyrosine related protein 1(tyrp2), Brachyury, Mesothelin, Epidermal growth factor receptor(EGFR), human epidermal growth factor receptor 2(HER-2), ERBB2, Wilms tumor protein(WT1), FAP, EpCAM, PD-L1, ACPP, CPT1A, IFNG, CD274, FOLR1, EPCAM, ICAM2, NCAM1, LRRC4, UNC5H2 LILRB2, CEACAM, Nectin-3 and a combination thereof. In addition, the target targeting protein may be an antibody specifically binding to a tumor associated antigen or a fragment thereof. In this case, linker 1 or 2, and the amino acid sequence recognized by a proteolytic enzyme are as described above.

<Structural Formula 16>

N terminal-mitochondrial outer membrane anchoring peptide-target targeting protein-C terminal

<Structural Formula 17>

N terminal-mitochondrial outer membrane anchoring peptide-ubiquitin or a fragment thereof-target targeting protein-C terminal

<Structural Formula 18>

N terminal-mitochondrial outer membrane anchoring peptide-linker 1-ubiquitin or a fragment thereof-target targeting protein-C terminal

<Structural Formula 19>

N terminal-mitochondrial outer membrane anchoring peptide-ubiquitin or fragment thereof-linker 2-target targeting protein-C terminal

<Structural Formula 20>

N terminal-mitochondrial outer membrane anchoring peptide-linker 1-ubiquitin or fragment thereof-linker 2-target targeting protein-C terminal

In the above Structural Formulae 16 to 20, the outer membrane anchoring peptide may be any one selected from the group consisting of TOM20, TOM70 and OM45. In addition, the target targeting protein, ubiquitin or a fragment thereof, and linker 1 or 2 are as described above.

One aspect of the present invention provides a polynucleotide encoding a fusion protein comprising a target targeting protein.

In addition, one aspect of the present invention provides a vector loaded with the polynucleotide encoding a fusion protein comprising a target targeting protein.

In addition, one aspect of the present invention provides a host cell in which a vector loaded with a polynucleotide encoding a fusion protein comprising the target targeting protein is introduced. The host cell may be a prokaryotic cell or a eukaryotic cell. In this case, preferably, the eukaryotic cell may be a strain from which an enzyme that degrades ubiquitin is removed.

In addition, one aspect of the present invention provides a method of preparing—modified mitochondria from the transformed cells by injecting a polynucleotide encoding the fusion protein into eukaryotic cells.

### MODE FOR THE INVENTION

Hereinafter, a preferred embodiment will be presented to help the understanding of the present invention. However, the following examples are provided only to easily understand the present invention, and the present invention is not limited to the following examples.

### I. Preparation of Fusion Protein Comprising Mitochondrial Outer Membrane Anchoring Peptide, Linker, Ubiquitin and Desired Protein

### Example 1. Preparation of Fusion Protein Comprising p53

### Example 1.1. Amplification of p53 Gene

In order to express the human p53 into a recombinant protein, total RNA was extracted from human epithelial cells, and cDNA was synthesized therefrom. Specifically, human dermal fibroblast cells were cultured in 10% serum medium under a condition of 5% carbon dioxide and 37° C. (1×10⁶ cells). Thereafter, the culture solution was removed and washed twice by adding a PBS buffer solution to the cells, and 0.5 ml of RNA extract (Trizol reagent, Thermo Fisher Scientific) was added directly. The mixture to which the RNA extract was added was stood at ambient temperature for 10 minutes, and then 0.1 ml of chloroform was added and stirred for 15 seconds, and then centrifuged at about 12,000×g for 10 minutes. Next, the separated supernatant was taken, and the same volume of isopropyl alcohol was added and centrifuged again at 12,000×g for 10 minutes. Thereafter, the liquid was removed and washed once with 75% ethanol, and the RNA was dried at ambient temperature.

About 50 ul of purified distilled water without RNAase was added, and the quantity and purity of RNA was measured using a spectrophotometer. In order to synthesize cDNA, 2 ug of purified total RNA was subjected to a binding reaction with oligo dT at 70° C. for 5 minutes. Thereafter, 10× reverse transcription reaction buffer solution, 10 mM dNTP, RNAse inhibitor and M-MLV reverse transcriptase (Enzynomics, Korea) were added, and cDNA synthesis reaction was performed at 42° C. for 60 minutes. Thereafter, the reverse transcriptase was inactivated by heating at 72° C. for 5 minutes, and then RNase H was added to remove single-stranded RNA, which was used as a template for the polymerase chain reaction of the p53 gene.

In order to obtain the gene of p53 in which the signal peptide sequence was removed from human dermal fibroblast cells, a primer (T2p53) encoding from the amino terminus glutamic acid and a primer (Xp53) encoding from the carboxyl terminus were synthesized, and then PCR was performed using the cDNA prepared above as a template. The sequence of each primer is as described in Table 1 below.

TABLE 1

| Primer | Sequence | SEQ ID NO. |
|---|---|---|
| T2p53 | 5′-AAA AAA CCG CGG TGG TGA GGA GCC GCA GTC AGA TCC TAG-3′ | SEQ ID NO: 1 |
| Xp53 | 5′-AAA AAA CTC GAG TGA GTC TGA GTC AGG CCC TTC TG-3′ | SEQ ID NO: 2 |

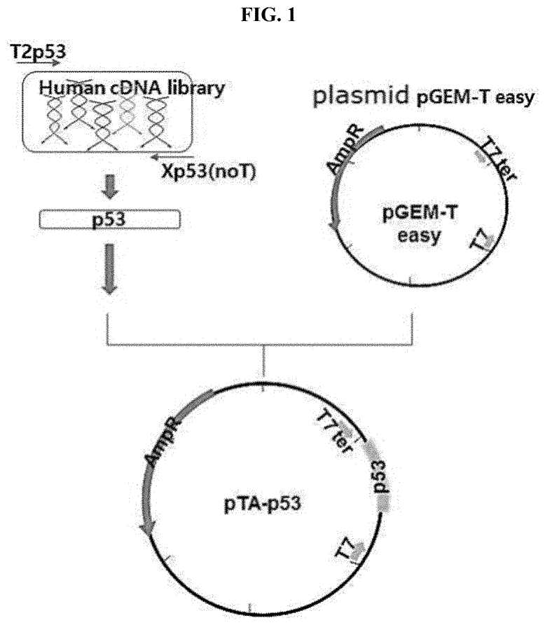

0.2 pmol T2p53 primer and 0.2 pmol Xp53 primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 40 cycles. After the reaction, the amplified DNA fragment of about 1.2 kbp was isolated by electrophoresis on 1% agarose gel, and then inserted into a pGEM-T easy (Promega, USA) vector using T4DNA ligase. As a result of sequencing the DNA thus obtained, it was confirmed that the cDNA encoding a human p53 protein was obtained. The obtained p53 gene was designated as pTA-p53, and the base sequence thereof is the same as the base sequence of SEQ ID NO: 3 (FIG. 1).

### Example 1.2. Preparation of E. coli Expression Vector for p53

### Example 1.2.1. Preparation of a Plasmid, pET15b-UB-p53

In order to prepare a p53 protein in a form to which ubiquitin is fused, the following expression vector was prepared. In order to obtain the ubiquitin gene, NdeUB primer and T2UB primer were prepared. The sequence of each primer is as described in Table 2 below.

TABLE 2

| Primer | Sequence | SEQ ID NO. |
|---|---|---|
| NdeUB | 5′-GGA TTC CAT ATG CAA CTT TTC GTC AAA ACT CTA AC-3′ | SEQ ID NO: 4 |
| T2UB | 5′-ATG ACC ACC GCG GAG TCT CAA CAC CAA-3′ | SEQ ID NO: 5 |

0.2 pmol NdeUB primer and 0.2 pmol T2UB primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 25 cycles to obtain the ubiquitin (UB) gene. The amplified ubiquitin gene was cleaved by the restriction enzymes NdeI and SacII, and the plasmid pTA-p53 was cleaved by the restriction enzymes SacII and XhoI. Thereafter, the DNA fragments of about 210 bp and 1,200 bp were obtained by electrophoresis on 2% agarose gel, respectively, and then inserted into a pET15b vector cleaved by the restriction enzymes NdeI and XhoI using a T4DNA ligase to obtain the plasmid pET15b-UB-p53 (FIG. 2). In this case, UB-p53 was represented by the base sequence of SEQ ID NO: 6.

E. coli BL21(DE3) strain was transformed using the plasmid pET15b-UB-p53. Thereafter, the transformed strain was cultured in a Luria-Bertani (LB) solid medium to which the antibiotic ampicillin was added, and then the colonies obtained herein were cultured in a LB liquid medium under a condition of 37° C. Thereafter, when the cell density reached about 0.2 absorbance at OD600, IPTG was added so that a final 1 mM concentration was made, and then the shaking culture was performed further for about 4 hours.

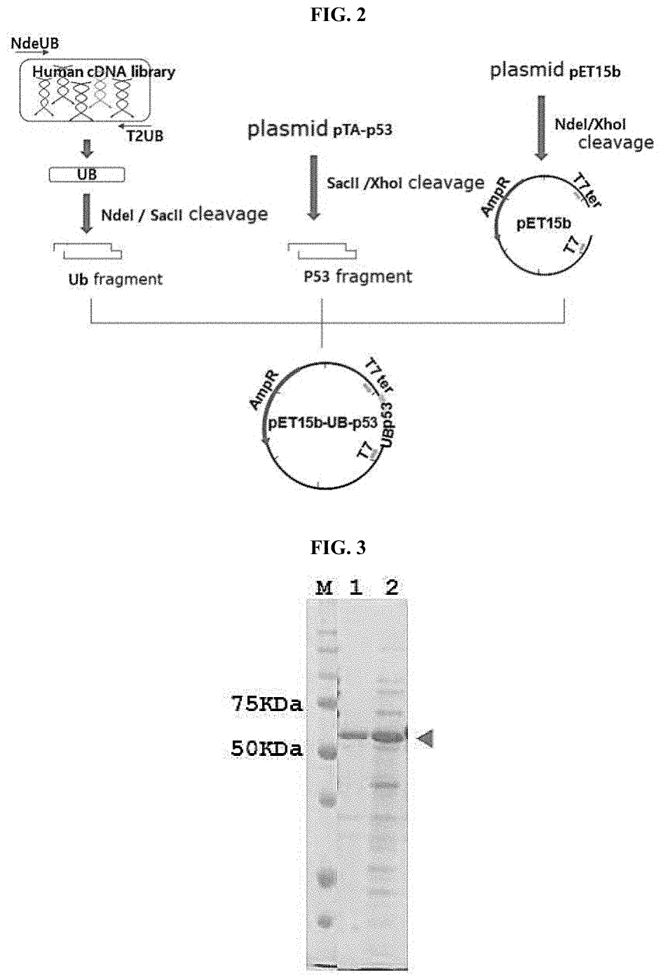

A portion of E. coli cells was obtained by centrifugation, and then the cells were crushed, and then SDS-polyacrylamide electrophoresis was performed. As shown in FIG. 3, it was confirmed that a ubiquitin-fused p53 protein having a size of about 60 kDa was expressed. In this case, lane M in FIG. 3 shows a protein molecular weight marker, and lane 1 shows the precipitate centrifuged after E. coli was crushed 4 hours after adding IPTG, and lane 2 shows the supernatant centrifuged after E. coli was crushed.

### Example 1.2.2. Preparation of a Plasmid, pET11C-TOM70-UB-p53

In order to prepare the p53 protein in the form to which TOM70 binding to the mitochondrial outer membrane and ubiquitin were fused, the expression vector capable of expressing p53 in the form to which TOM70 and ubiquitin were fused was prepared. In order to obtain TOM70 and ubiquitin genes, NdeTOM70 primer, TOM70-AS primer, TOM70UB-S primer and T2UB-AS primer were prepared. The sequence of each primer is as described in Table 3 below.

TABLE 3

| Primer | Sequence | SEQ ID NO. |
|---|---|---|
| NdeTOM70 | 5′-GAA TTC CAT ATG AAA AGT TTT ATA ACT CGG AAT AAA | SEQ ID NO: 7 |
| | ACT GCA ATT TTC GCA ACT GTT GC-3′ | |
| TOM70-AS | 5′-GGT GCA TAC TAC TAT TAT CAAA CTT TTC GTC AAA ACT | SEQ ID NO: 8 |
| | C-3′ | |
| TOM70UB-S | 5′-GGC TAC GT ATT TAT TTC CAA CTT TTC GTC AAA ACT C-3′ | SEQ ID NO: 9 |
| T2UB-AS | 5′-GGC ACC ACC GCG GAG TCT CAA CAC 3′ | SEQ ID NO: 10 |

In order to obtain the TOM70 gene, 0.2 pmol NdeTOM70 primer and 0.2 pmol TOM70-AS primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 25 cycles to obtain a TOM70 gene. The amplified DNA fragment was referred to as N-TOM70. The plasmid pET15b-UB-p53 obtained in Example 1.2.1. above was used as a template, and 0.2 pmol TOM70UB-S primer and 0.2 pmol T2UB-AS primer were added, and dNTP 0.2 nM, lx AccuPrime Taq DNA polymerase reaction buffer solution (Invitrogen, USA) and 1 unit of AccuPrime Taq DNA polymerase were mixed. Thereafter, in a polymerase chain reaction apparatus, amplification reactions of 95° C. for 40 seconds, 58° C. for 30 seconds, 72° C. for 1 minute were performed at 25 cycles to obtain a UB gene. The amplified DNA fragment was referred to as C-UB.

The amplified DNA N-TOM70 and C-UB were used as templates, and 0.2 pmol NdeTOM70 primer, 0.2 pmol T2UB-AS primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 25 cycles to obtain the ubiquitin gene TOM70-UB to which the amplified TOM70 was fused.

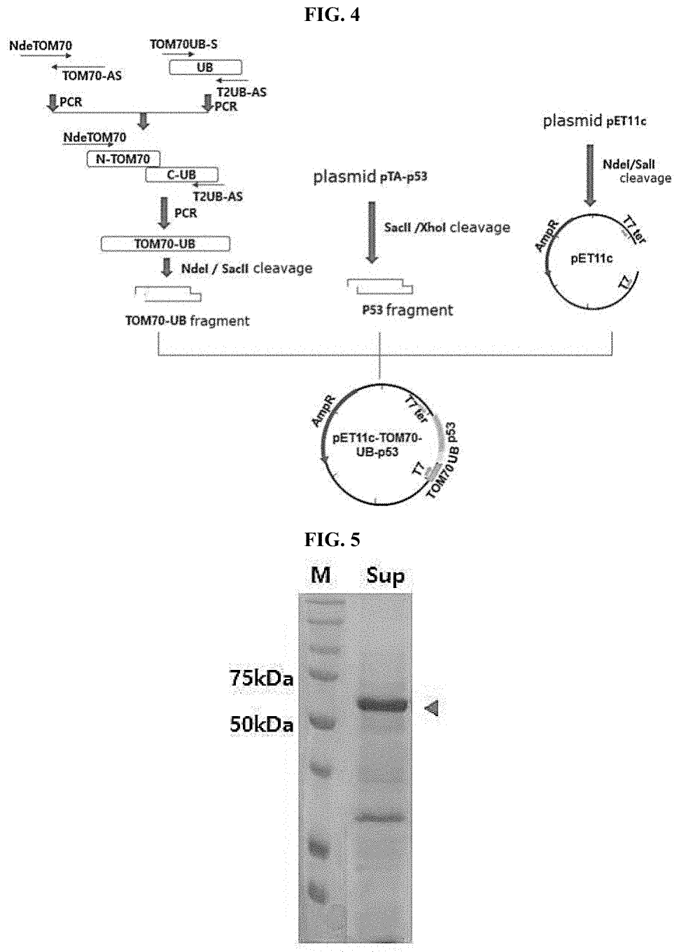

The amplified TOM70-UB gene was cleaved by the restriction enzymes NdeI and SacII, and the plasmid pTA-p53 was cleaved by the SacII and XhoI, and the DNA fragments of 330 bp and 1,500 bp were obtained by electrophoresis on 2% agarose gel, respectively. Thereafter, it was inserted into a pET11c vector cleaved by the restriction enzymes NdeI and SalI using a T4DNA ligase to obtain the plasmid pET11c-TOM70-UB-p53 (FIG. 4). In this case, TOM70-UB-p53 was represented by the base sequence of SEQ ID NO: 11.

E. coli BL21(DE3) strain was transformed using a plasmid pET11c-TOM70-UB-p53. Thereafter, the transformed strain was cultured in a Luria-Bertani (LB) solid medium to which the antibiotic ampicillin was added, and then the colonies obtained herein were cultured in a LB liquid medium in a 37° C. shaking incubator. Thereafter, when the cell density reached about 0.2 absorbance at OD600, IPTG was added so that a final 1 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 5, it was confirmed that p53 protein having a size of about 62 kDa in the form to which TOM70 and ubiquitin were fused was expressed. In this case, lane M shows a protein molecular weight marker, and lane 1 shows the supernatant centrifuged after E. coli was crushed 4 hours after adding IPTG.

### Example 1.2.3. Preparation of a Plasmid, pET11c-TOM70-(GGGGS)3-UB-p53

In order to prepare a p53 protein in the form to which TOM70 binding to the mitochondrial outer membrane, a linker (GGGGSGGGGSGGGGS (SEQ ID NO: 70)) and ubiquitin were fused, an expression vector capable of expressing a p53 protein in the form to which TOM70, the linker, and ubiquitin were fused was prepared. In order to obtain a linker gene bound to TOM70, TOM70(G)3-AS primer, (G)3UB-S primer and Xp53 (noT) primer were prepared. The sequence of each primer is as described in Table 4 below.

TABLE 4

| Primer | Sequence | SEQ ID NO. |
|---|---|---|
| TOM70(G)₃- | 5′-GCC CCC GGA TCC TCC ACC CCC GCT TCC GCC ACC TCC ATA | SEQ ID NO: 12 |
| AS | ATA GT AGT ATG CAC CAA TAG-3′ | |
| (G)₃UB-S | 5′-GGT GGA GGA TCC GGG GGC GC GGA AGC CAA ATC-3′ | SEQ ID NO: 13 |
| Xp53(noT) | 5′-AAA AAA CTC GAG GTC TGA GTC AGG CCC TTC TG-3′ | SEQ ID NO: 14 |

The plasmid pET11c-TOM70-UB-p53 obtained in Example 1.2.2. above was used as a template, and 0.2 pmol NdeTOM70 primer and 0.2 pmol TOM70(G)3-AS primer were added, and dNTP 0.2 nM, 1× AccuPrime Taq DNA polymerase reaction buffer solution (Invitrogen, USA) and 1 unit of AccuPrime Taq DNA polymerase were mixed. Thereafter, in a polymerase chain reaction apparatus, amplification reactions of 95° C. for 40 seconds, 58° C. for 30 seconds, 72° C. for 1 minute were performed at 25 cycles to obtain a gene TOM70-G3 in which a gene TOM70 and a linker were bound. In addition, the plasmid pET15b-UB-p53 obtained in Example 1.2.1. above was used as a template, and 0.2 pmol (G)3UB-S primer and 0.2 pmol Xp53 (noT) primer were mixed with dNTP 0.2 nM, 1× AccuPrime Taq DNA polymerase reaction buffer solution (Invitrogen, USA) and 1 unit of AccuPrime Taq DNA polymerase.

Thereafter, in a polymerase chain reaction apparatus, amplification reactions of 95° C. for 40 seconds, 58° C. for 30 seconds, 72° C. for 1 minute were performed at 25 cycles to obtain UB-p53, p53 fused with the gene ubiquitin. The amplified TOM70-G3 gene was cleaved by the restriction enzymes NdeI and BamHI, and the amplified UB-p53 gene was cleaved by BamHI and XhoI, and the DNA fragments of 100 bp and 1,500 bp were obtained by electrophoresis on 2% agarose gel, respectively. Thereafter, it was inserted into a pET11c vector cleaved by the restriction enzymes NdeI and SalI using a T4DNA ligase to obtain the plasmid pET11c-TOM70-(GGGGS)3-UB-p53 (FIG. 6). In this case, TOM70-(GGGGS)3-UB-p53 was represented by the base sequence of SEQ ID NO: 15.

E. coli BL21(DE3) strain was transformed using the plasmid pET11c-TOM70-(GGGGS)3-UB-p53. Thereafter, the transformed strain was cultured in a Luria-Bertani (LB) solid medium to which the antibiotic ampicillin was added, and then the colonies obtained herein were cultured in a LB liquid medium under a condition of 37° C. Thereafter, when the cell density reached about 0.2 absorbance at OD600, IPTG was added so that a final 1 mM concentration was made, and then the shaking culture was performed further for about 4 hours.

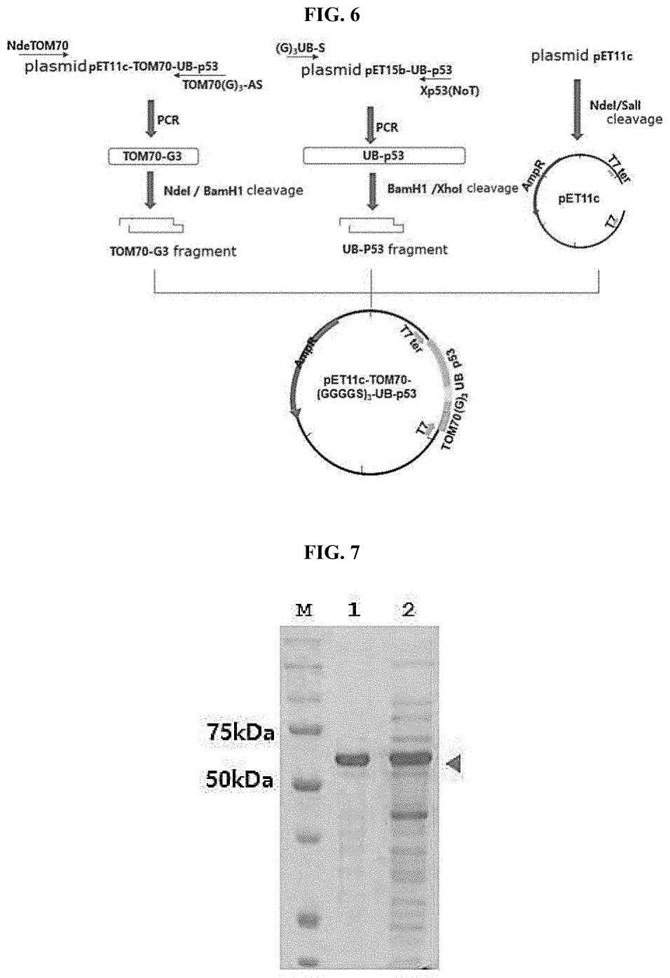

A portion of E. coli cells was obtained by centrifugation, and then the cells were crushed, and then SDS-polyacrylamide electrophoresis was performed. As shown in FIG. 7, it was confirmed that p53 protein having a size of about 62 kDa in the form to which TOM70, the linker and ubiquitin were fused was expressed. In this case, lane M shows a protein molecular weight marker, lane 1 shows the precipitate centrifuged after E. coli was crushed 4 hours after adding IPTG, and lane 2 shows the supernatant centrifuged after crushing E. coli.

### Example 1.2.4. Preparation of a Plasmid, pET11c-TOM70-(GGGGS)3-p53

In order to prepare a p53 protein in the form to which TOM70 binding to the mitochondrial outer membrane and a linker (GGGGSGGGGSGGGGS) were fused, an expression vector capable of expressing a p53 protein in the form to which TOM70 and the linker were fused was prepared. In order to obtain a p53 gene to which TOM70 and the linker were fused, a primer (B(G)3p53) was prepared. The sequence of each primer is as described in Table 5 below.

TABLE 5

| Primer | Sequence | SEQ ID NO. |
|---|---|---|
| B(G)₃p53 | 5′-GGT GGA GGA TCC GGG GGC GGC GGA AGC GAG GAG CCG | SEQ ID NO: 16 |
| | CAG TCA GAT CCT AGC-3′ | |

The plasmid pET11c-TOM70-UB-p53 obtained in Example 1.2.2. above was used as a template, and 0.2 pmol NdeTOM70 primer and 0.2 pmol TOM70(G)3-AS primer were added, and dNTP 0.2 nM, 1× AccuPrime Taq DNA polymerase reaction buffer solution (Invitrogen, USA) and 1 unit of AccuPrime Taq DNA polymerase were mixed. Thereafter, in a polymerase chain reaction apparatus, amplification reactions of 95° C. for 40 seconds, 58° C. for 30 seconds, 72° C. for 1 minute were performed at 25 cycles to obtain a gene TOM70. The amplified DNA fragment was referred to as TOM70-G3.

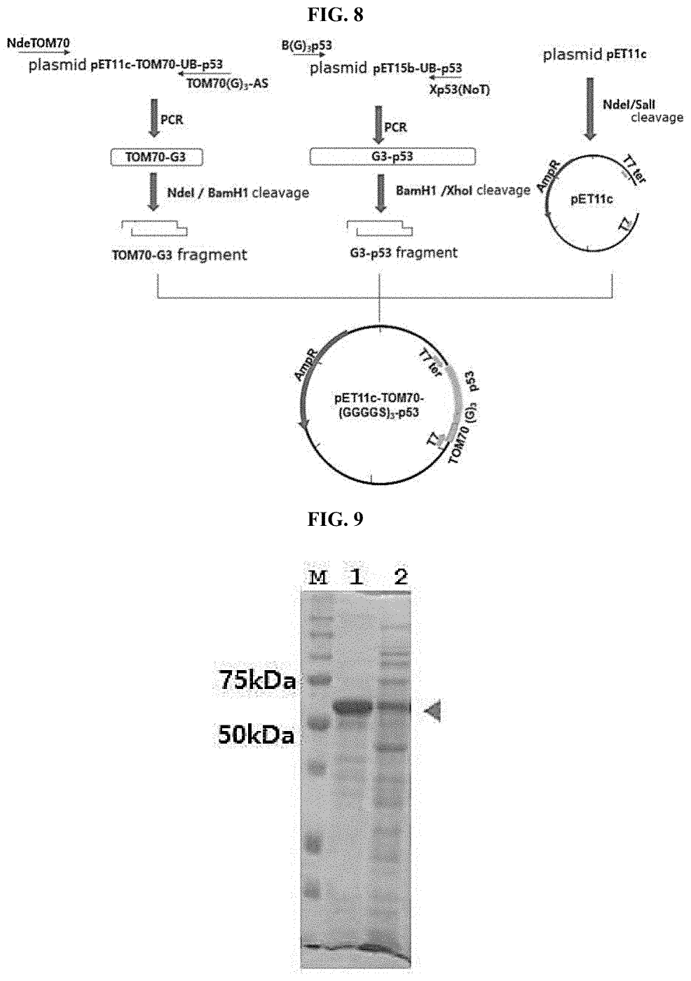

The plasmid pET15b-UB-p53 obtained in Example 1.2.1. above was used as a template, and 0.2 pmol B(G)3p53 primer and 0.2 pmol Xp53 (noT) primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 25 cycles. The amplified DNA fragment was referred to as G3-p53. The amplified DNA fragment, TOM70-G3, was cleaved by NdeI and BamHI, and the DNA fragment G3-53 was cleaved by the restriction enzymes BamHI and XhoI. Then, the DNA fragments of about 150 bp and 1,300 bp were obtained by electrophoresis on 2% agarose gel, respectively, and then inserted into a pET11c vector cleaved by the restriction enzymes NdeI and SalI using a T4DNA ligase to obtain the plasmid pET11c-TOM70-(GGGGS)3-p53 (FIG. 8). In this case, TOM70-(GGGGS)3-p53 was represented by the base sequence of SEQ ID NO: 17.

E. coli BL21(DE3) strain was transformed using the plasmid pET11c-TOM70-(GGGGS)3-p53. Thereafter, the transformed strain was cultured in a Luria-Bertani (LB) solid medium to which the antibiotic ampicillin was added, and then the colonies obtained herein were cultured in a LB liquid medium in a 37° C. shaking incubator. Thereafter, when the cell density reached about 0.2 absorbance at OD600, IPTG was added so that a final 1 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 9, it was confirmed that a p53 protein having a size of about 60 kDa in the form to which TOM70 was fused was expressed. In this case, lane M shows a protein molecular weight marker, lane 1 shows the precipitate centrifuged after E. coli was crushed 4 hours after adding IPTG, and lane 2 shows the supernatant centrifuged after crushing E. coli.

### Example 1.2.5. pET15b-UB-p53-TOM7

In order to prepare a p53 protein in the form to which ubiquitin and TOM7 binding to the mitochondrial outer membrane were fused, an expression vector capable of expressing p53 in a form to which ubiquitin, p53 and TOM were fused in the order was prepared. In order to obtain a p53 gene to which TOM7 and ubiquitin were fused, Xp53(noT) primer, XTOM7 primer and LTOM7 primer were prepared. The sequence of each primer is as described in Table 6 below.

TABLE 6

| Primer | Sequence | SEQ ID NO. |
|---|---|---|
| Xp53(noT) | 5′-AAA AAA CTC GAG GTC TGA GTC AGG CCC TTC TG-3′ | SEQ ID NO: 18 |
| XTOM7 | 5′-AAA AAA CTC GAG ttt gcc att cgc tgg ggc ttt atc-3′ | SEQ ID NO: 19 |
| LTOM7 | 5′-AAA AAA GTC GAC TTA TCC CCA AAG TAG GCT CAA AAC | SEQ ID NO: 20 |
| | AG-3′ | |

The plasmid pET15b-UB-p53 obtained in Example 1.2.1. above was used as a template, and 0.2 pmol NdeUB primer and 0.2 pmol Xp53(noT) primer were added, and dNTP 0.2 nM, 1× AccuPrime Taq DNA polymerase reaction buffer solution (Invitrogen, USA) and 1 unit of AccuPrime Taq DNA polymerase were mixed. Thereafter, in a polymerase chain reaction apparatus, amplification reactions of 95° C. for 40 seconds, 58° C. for 30 seconds, 72° C. for 1 minute were performed at 25 cycles to obtain a gene UB-p53. In addition, cDNA prepared above was used as a template, and 0.2 pmol XTOM7 primer and 0.2 pmol LTOM7 primer were mixed with dNTP 0.2 nM, 1× AccuPrime Taq DNA polymerase reaction buffer solution (Invitrogen, USA) and 1 unit of AccuPrime Taq DNA polymerase.

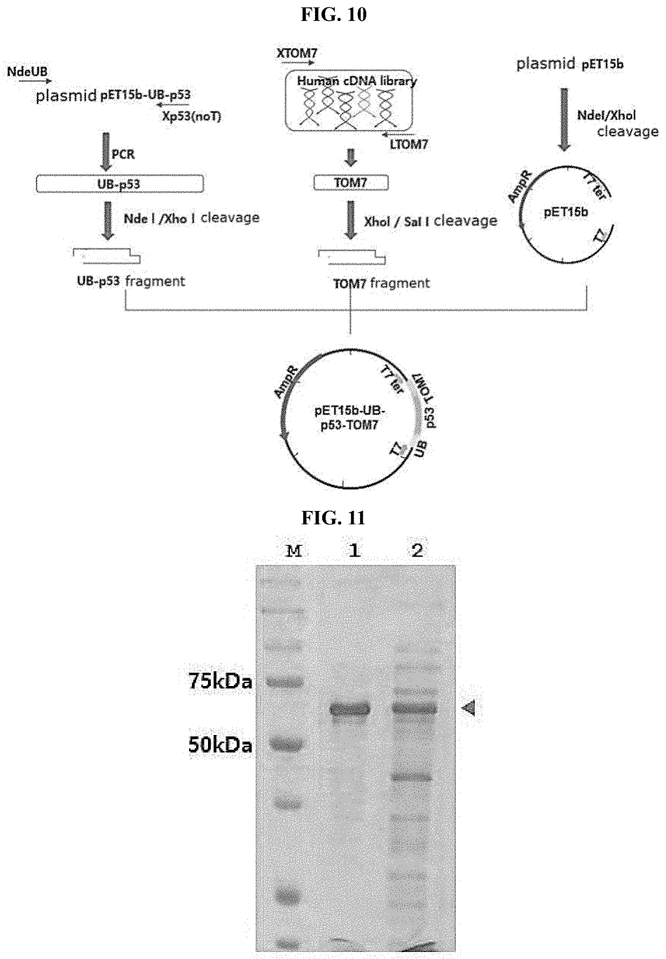

Thereafter, in a polymerase chain reaction apparatus, amplification reactions of 95° C. for 40 seconds, 58° C. for 30 seconds, 72° C. for 1 minute were performed at 40 cycles to obtain a gene TOM7. The amplified DNA fragment, UB-p53, was cleaved by the restriction enzymes, NdeI and XhoI, and the amplified TOM7 gene was cleaved by the restriction enzymes XhoI and SalI. The DNA fragments of about 1,500 bp and 150 bp were obtained by electrophoresis on 2% agarose gel, respectively, and then inserted into a pET15b vector cleaved by the restriction enzymes NdeI and XhoI using a T4DNA ligase to obtain the plasmid pET15b-UB-p53-TOM7 (FIG. 10). In this case, UB-p53-TOM7 was represented by the base sequence of SEQ ID NO: 21.

E. coli BL21(DE3) strain was transformed using the plasmid pET15b-UB-p53-TOM7. Thereafter, the transformed strain was cultured in a Luria-Bertani (LB) solid medium to which the antibiotic ampicillin was added, and then the colonies obtained herein were cultured in a LB liquid medium under a condition of 37° C. Thereafter, when the cell density reached about 0.2 absorbance at OD600, IPTG was added so that a final 0.5 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 11, it was confirmed that a p53 protein having a size of about 60 kDa in the form to which ubiquitin and TOM7 were fused was expressed. In this case, lane M shows a protein molecular weight marker, lane 1 shows the precipitate centrifuged after E. coli was crushed 4 hours after adding IPTG, and lane 2 shows the supernatant centrifuged after crushing E. coli.

### Example 1.2.6. Construction of a Mammalian Expression Vector, pCMV-p53-myc/His

An expression vector for animal cells capable of expressing p53 was prepared. In order to obtain a p53 gene, Rp53 primer was prepared. The sequence of each primer is as described in Table 7 below.

TABLE 7

| Primer | Sequence | SEQ ID NO. |
|---|---|---|
| Rp53 | 5′-AAA AAA GAA TTC ATG GTC TGA GTC AGG CCC TTC TG-3′ | SEQ ID NO: 23 |

The plasmid pET-UB-p53 obtained in Example 1.2.1. above was used as a template, and 0.2 pmol Rp53 primer and 0.2 pmol Xp53(noT) primer were mixed with dNTP 0.2 nM, lx AccuPrime Taq DNA polymerase reaction buffer solution (Invitrogen, USA) and 1 unit of AccuPrime Taq DNA polymerase were mixed. Thereafter, in a polymerase chain reaction apparatus, amplification reactions of 95° C. for 40 seconds, 58° C. for 30 seconds, 72° C. for 1 minute were performed at 25 cycles to obtain a gene p53.

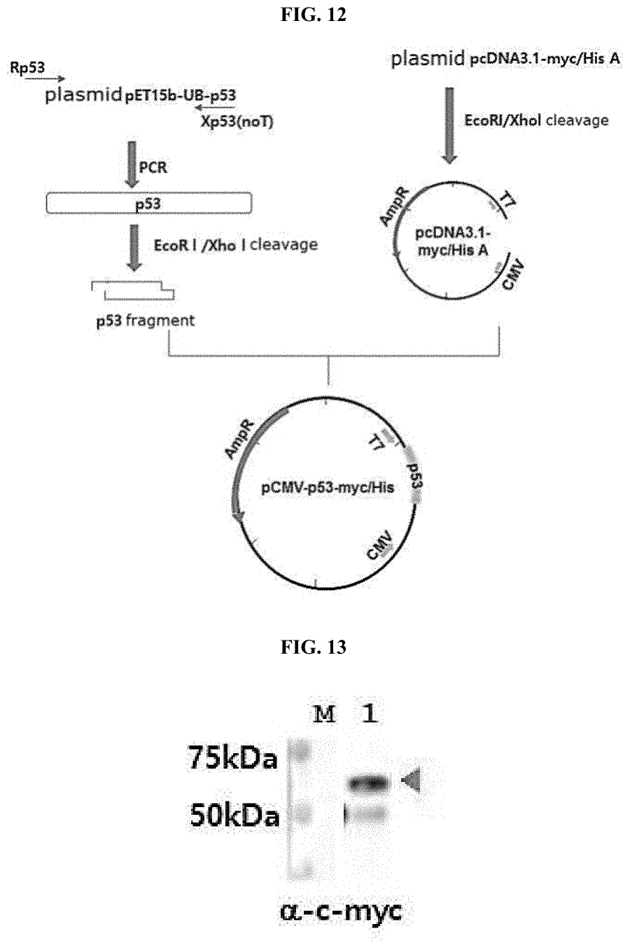

The amplified p53 gene was cleaved by the restriction enzymes EcoRI and XhoI, and the DNA fragment of about 1,300 bp was obtained by electrophoresis on 2% agarose gel, and then it was inserted into a pcDNA3.1-myc/His A vector cleaved by the restriction enzymes EcoRI and XhoI using a T4DNA ligase to obtain the plasmid pCMV-p53-myc/His (FIG. 12). In this case, p53-myc/His was represented by the base sequence of SEQ ID NO: 23.

It was transfected into an animal cell CHO using the plasmid pCMV-p53-myc/His, and the cells was crushed, and then SDS-polyacrylamide electrophoresis was performed, and it was shown by Western blot using an anti-c-myc antibody. As shown in FIG. 13, it was confirmed that a p53 protein having a size of about 55 kDa was expressed. In this case, lane M shows a protein molecular weight marker, and lane 1 shows that it was transfected into an animal cell CHO, and the cells was crushed, and then SDS-polyacrylamide electrophoresis was performed, and then it was confirmed by Western blot using an anti-c-myc antibody.

### Example 1.3. Isolation and Purification of Fusion Protein Comprising p53

### Example 1.3.1. Isolation and Purification of Recombinant TOM70-(GGGGS)3-p53 Protein Derived from E. coli

E. coli BL21(DE3) production strain expressing the recombinant TOM70-(GGGGS)3-p53 protein was inoculated into a LB liquid medium, and cultured under a condition of 37° C. Thereafter, when the absorbance reached 0.4 at OD600, 0.5 mM IPTG was added, and the shaking culture was performed further for 4 hours to express the TOM70-(GGGGS)3-p53 protein.

After the culture was completed, the cells were recovered using centrifugation, and the recovered cells were washed once using PBS, and then the cells were suspended using a PBS solution, and the suspended cells were subjected to a crushing process using a sonicator. The crushed cells were centrifuged using a high speed centrifuge, and then insoluble fractions were recovered, and the recovered insoluble fractions were washed three times using 50 mM Tris, 100 mM ethylenediaminetetraacetic acid (EDTA) pH 8.0 solution. Thereafter, it was dissolved in 6 M guanidine, 100 mM sodium phosphate, 10 mM Tris pH 8.0 solution and filtered using a 0.45 μm filter, and then loaded on a pre-packed nickel chromatography column to perform primary purification.

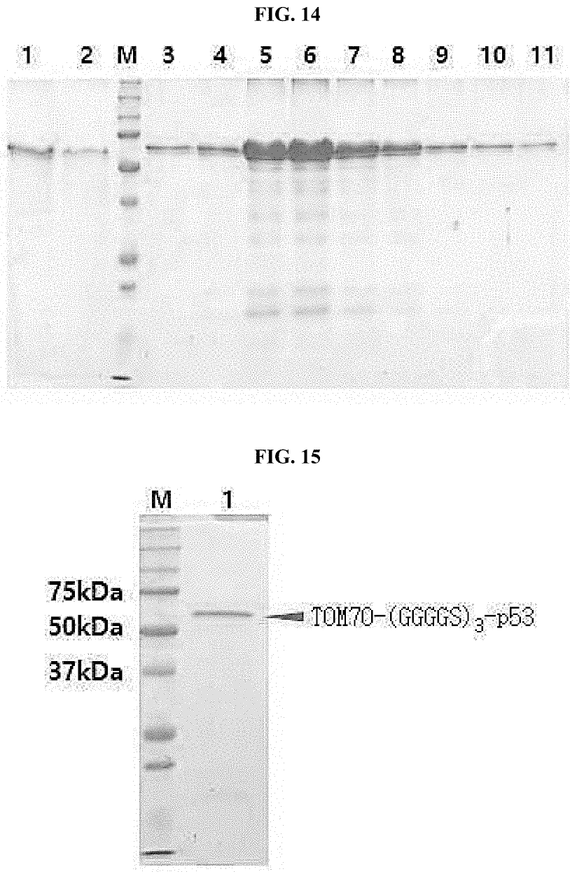

The solution comprising the TOM70-(GGGGS)3-p53 protein was loaded, and then until the impurities unbound were not detected, a washing solution was flowed using 8 M urea, 50 mM sodium phosphate, 500 mM NaCl, 10 mM imidazole, pH 8.0 solution, and the protein was eluted using 8 M urea, 50 mM sodium phosphate, 500 mM NaCl, 500 mM imidazole, pH 8.0 solution while changing the imidazole concentration to 50 mM, 100 mM, 250 mM, 500 mM (FIG. 14). In this case, lane M in FIG. 14 shows a protein molecular weight marker, and lane 1 shows a nickel affinity chromatography loading sample. Lane 2 shows that it was not bound to a nickel affinity resin. Lanes 3 to 4 show the results of elution with 8 M UREA/50 mM Na-phosphate/500 mM NaCl/50 mM imidazole solution. Lanes 5 to 7 show the results of elution with 8M UREA/50 mM Na-phosphate/500 mM NaCl/100 mM Imidazole solution. Lanes 8 to 9 show the results of elution with 8M UREA/50 mM Na-phosphate/500 mM NaCl/250 mM Imidazole solution. Lanes 10 to 11 show the results of elution with 8M UREA/50 mM Na-phosphate/500 mM NaCl/500 mM Imidazole solution.

The eluted solution recovered from the nickel chromatography was solution-exchanged with PBS using the principle of osmotic pressure. After the solution exchange was completed, the eluted solution was subjected to centrifugation to recover the supernatant, and a protein amount of the recovered eluted solution was measured by protein quantification method and confirmed using SDS-PAGE. As shown in FIG. 15, after the confirmation was completed, the TOM70-(GGGGS)3-p53 protein was quenched with the liquid nitrogen and stored in a cryogenic freezer at −80° C. In this case, lane M shows a protein molecular weight marker, and lane 1 shows the TOM70-(GGGGS)3-p53 protein obtained after dialysis in PBS buffer solution.

### Example 1.3.2. Isolation and Purification of Recombinant TOM70-(GGGGS)3-UB-p53 Protein Derived from E. coli

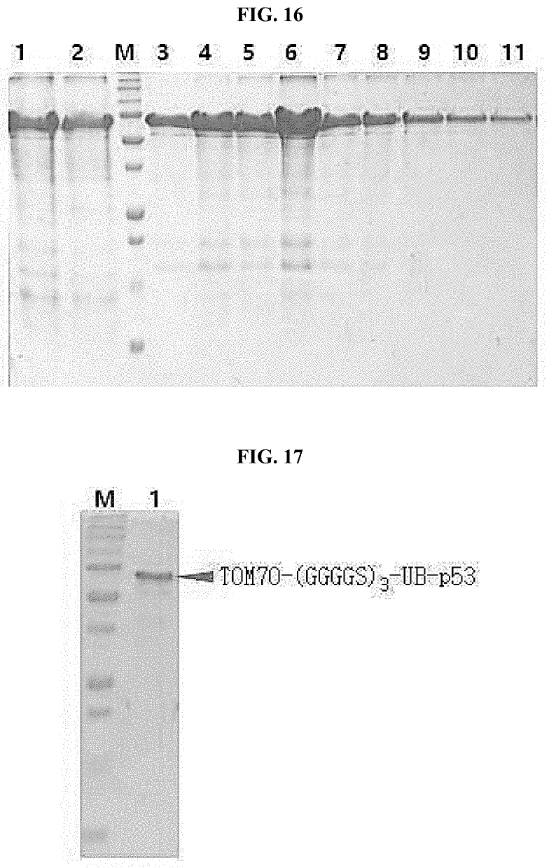

E. coli expressing the TOM70-(GGGGS)3-UB-p53 recombinant protein was used to isolate and purify the TOM70-(GGGGS)3-UB-p53 protein in the same method as in Example 1.3.1. As a result, the TOM70-(GGGGS)3-UB-p53 protein was eluted (FIG. 16). In this case, lane M in FIG. 16 shows a protein molecular weight marker, and lane 1 shows a nickel affinity chromatography loading sample. Lane 2 shows that it was not bound to a nickel affinity resin. Lane 3 shows the results of elution with 8M UREA/50 mM Na-phosphate/500 mM NaCl/50 mM Imidazole solution. Lanes 4 to 7 show the results of elution with 8M UREA/50 mM Na-phosphate/500 mM NaCl/100 mM Imidazole solution. Lanes 8 to 11 show the results of elution with 8M UREA/50 mM Na-phosphate/500 mM NaCl/250 mM Imidazole solution.

The protein amount of the recovered eluted solution was measured by protein quantification method and confirmed using SDS-PAGE. As shown in FIG. 17, after the confirmation was completed, the TOM70-(GGGGS)3-UB-p53 protein was quenched with the liquid nitrogen and stored in a cryogenic freezer at −80° C. In this case, lane M in FIG. 17 shows a protein molecular weight marker, and lane 1 shows the TOM70-(GGGGS)3-UB-p53 protein obtained after dialysis in PBS buffer solution.

### Example 1.3.3. Isolation and Purification of Recombinant UB-p53 Protein Derived from E. coli

BL21(DE3) production strain expressing the UB-p53 protein in the mature form to which ubiquitin was fused was innoculated into a LB liquid medium, and cultured in a shaking incubator at 37° C. When the absorbance reached 0.4 at OD600, 0.5 mM IPTG was added, and the shaking culture was performed further for 4 hours to express the UB-p53 protein in the mature form to which ubiquitin was fused.

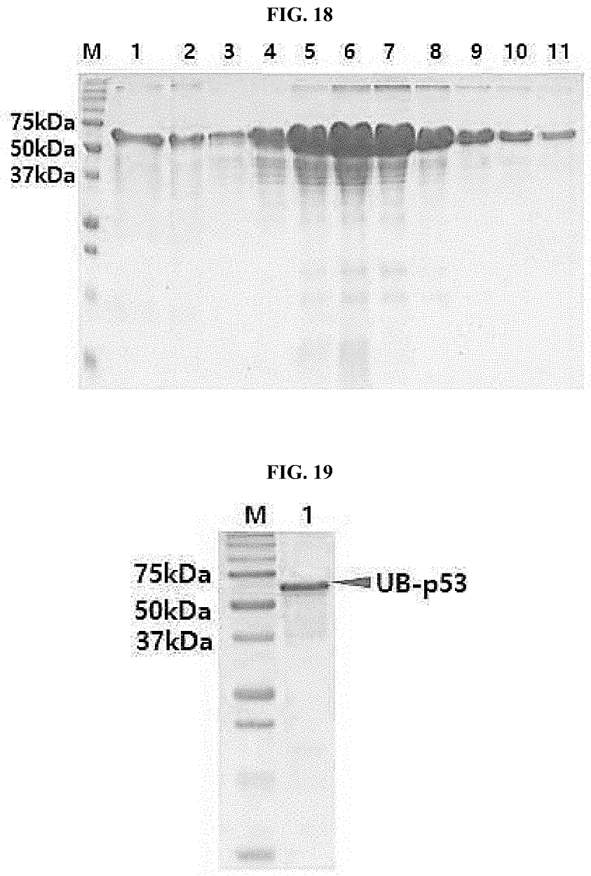

Then, the UB-p53 protein was isolated and purified in the same method as in Example 1.3.1. As a result, the UB-p53 protein was eluted (FIG. 18). In this case, lane M in FIG. 18 shows a protein molecular weight marker, and lane 1 shows a nickel affinity chromatography loading sample. Lane 2 shows that it was not bound to a nickel affinity resin. Lane 3 shows the results of elution with 8M UREA/50 mM Na-phosphate/500 mM NaCl/50 mM Imidazole solution. Lanes 4 to 6 show the results of elution with 8M UREA/50 mM Na-phosphate/500 mM NaCl/100 mM Imidazole solution. Lane 7 to 9 show the results of elution with 8M UREA/50 mM Na-phosphate/500 mM NaCl/250 mM Imidazole solution. Lanes 10 to 11 show the results of elution with 8M UREA/50 mM Na-phosphate/500 mM NaCl/500 mM Imidazole solution.

The protein amount of the recovered eluted solution was measured by protein quantification method and confirmed using SDS-PAGE. As shown in FIG. 19, after the confirmation was completed, the UB-p53 protein was quenched with the liquid nitrogen and stored in a cryogenic freezer at −80° C. In this case, lane M in FIG. 19 shows a protein molecular weight marker, and lane 1 shows the UB-p53 protein obtained after dialysis in PBS buffer solution.

### Example 1.3.4. Isolation and Purification of Recombinant UB-p53-TOM7 Protein Derived from E. coli

E. coli BL21(DE3) production strain expressing the UB-p53-TOM7 protein in the mature form to which ubiquitin was fused was innoculated into a LB liquid medium, and cultured under a condition of 37° C. When the absorbance reached 0.4 at OD600, 0.5 mM IPTG was added, and the shaking culture was performed further for 4 hours to express the UB-p53-TOM7 protein in the mature form to which ubiquitin was fused.

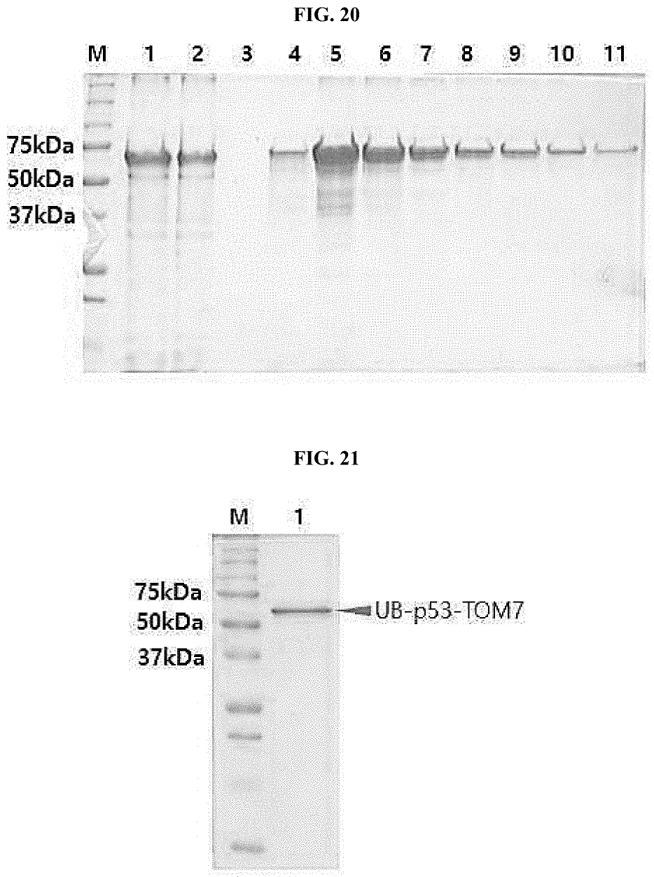

Then, the UB-p53-TOM7 protein was isolated and purified in the same method as in Example 1.3.1. As a result, the UB-p53-TOM7 protein was eluted (FIG. 20). In this case, lane M in FIG. 20 shows a protein molecular weight marker, and lane 1 shows a nickel affinity chromatography loading sample. Lane 2 shows that it was not bound to a nickel affinity resin. Lane 3 shows the results of elution with 8M UREA/50 mM Na-phosphate/500 mM NaCl/10 mM Imidazole solution. Lane 4 shows the results of elution with 8M UREA/50 mM Na-phosphate/500 mM NaCl/50 mM Imidazole solution. Lanes 5 to 7 show the results of elution with 8M UREA/50 mM Na-phosphate/500 mM NaCl/100 mM Imidazole solution. Lanes 8 to 9 show the results of elution with 8M UREA/50 mM Na-phosphate/500 mM NaCl/250 mM Imidazole solution. Lanes 10 to 11 show the results of elution with 8M UREA/50 mM Na-phosphate/500 mM NaCl/500 mM Imidazole solution.

The protein amount of the recovered eluted solution was measured by protein quantification method and confirmed using SDS-PAGE. As shown in FIG. 21, after the confirmation was completed, the UB-p53 protein was quenched with the liquid nitrogen and stored in a cryogenic freezer at −80° C. In this case, lane M in FIG. 21 shows a protein molecular weight marker, and lane 1 shows the UB-p53-TOM7 protein obtained after dialysis in PBS buffer solution.

### Example 2. Preparation of Fusion Protein Comprising Granzyme B

### Example 2.1. Amplification of Granzyme B Gene

In order to express the human Granzyme B into a recombinant protein, total RNA was extracted from human natural killer cells, and cDNA was synthesized therefrom. Specifically, human natural killer cells were cultured in 10% serum medium under a condition of 5% carbon dioxide and 37° C. (1×10⁶ cells). Thereafter, the RNA was obtained in the same method as in Example 1.1., and then it was used as a template for the polymerase chain reaction of the Granzyme B gene.

In order to obtain the gene of Granzyme B in which the signal peptide sequence was removed from human natural killer cells, T2GZMB primer encoding from the amino terminus isoleucine and XGZMB(noT) primer encoding from the carboxyl terminus were synthesized, and then PCR was performed using the cDNA prepared above as a template. The sequence of each primer is as described in Table 8 below.

TABLE 8

| Primer | Sequence | SEQ ID NO. |
|---|---|---|
| T2GZMB | 5′-AAA AAA CCG CGG TGG TAT CAT CGG GGG ACA TGA GGC | SEQ ID NO: 24 |
| | ACA TGA GGC CAA GCC-3′ | |
| XGZMB(noT) | 5′-AAA AAA CTC GAG GTA GCG TTT CAT GGT TTT CTT TAT | SEQ ID NO: 25 |
| | CC-3′ | |

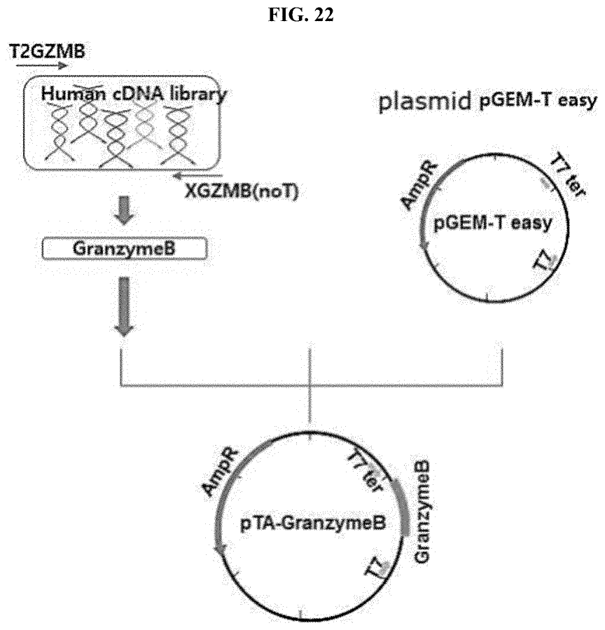

The cDNA prepared above was used as a template, and 0.2 pmol T2GZMB primer and 0.2 pmol XGZMB(noT) primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 40 cycles. After the reaction, the amplified DNA fragment of about 700 bp was isolated by electrophoresis on 1% agarose gel, and then inserted into a pGEM-T easy (Promega, USA) vector using a T4DNA ligase. As a result of sequencing the DNA thus obtained, it was confirmed that the cDNA encoding a human Granzyme B protein was obtained. The obtained Granzyme B gene was designated as pTA-Granzyme B, and the Granzyme B gene was represented by the base sequence of SEQ ID NO: 26 (FIG. 22).

### Example 2.2. Preparation of an E. coli Expression Vector for Granzyme B Protein

### Example 2.2.1. Preparation of a Plasmid, pET11c-TOM70-(GGGGS)3-UB-Granzyme B

In order to prepare a Granzyme B protein in the form to which TOM70 binding to the mitochondrial outer membrane, a linker (GGGGSGGGGSGGGGS) and ubiquitin were fused, the expression vector capable of expressing Granzyme B in the form to which TOM70, the linker and ubiquitin were fused was prepared.

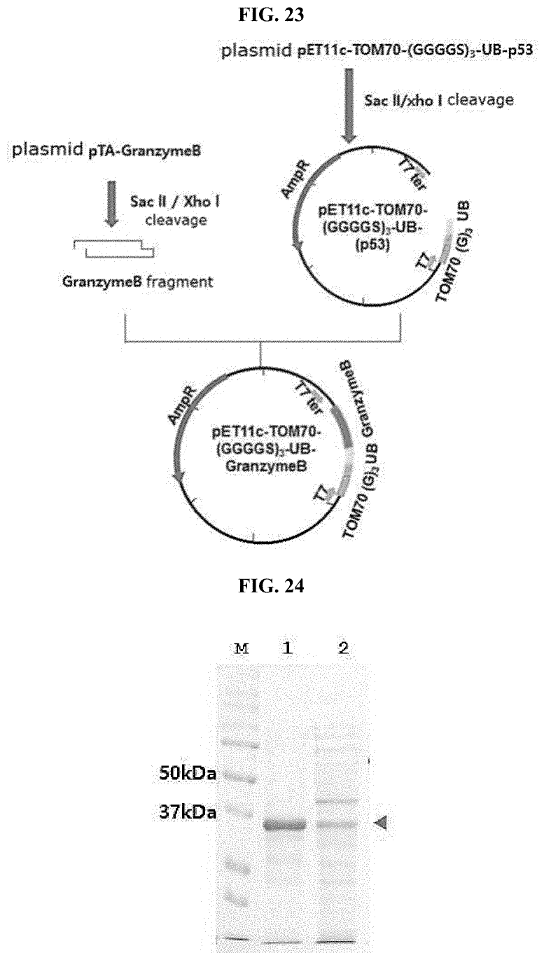

The plasmid pTA-GranzymeB gene obtained in Example 2.1. above was cleaved by the restriction enzymes SacII and XhoI, and and the DNA fragment of about 700 bp was obtained by electrophoresis on 2% agarose gel. Thereafter, it was inserted into a pET11c-TOM70-(GGGGS)3-UB-(p53) vector cleaved by the restriction enzymes SacII and XhoI using a T4DNA ligase to obtain the plasmid pET11c-TOM70-(GGGGS)3-UB-Granzyme B (SEQ ID NO: 27) (FIG. 23).

E. coli BL21(DE3) strain was transformed using a plasmid pET11c-TOM70-(GGGGS)3-UB-Granzyme B. Thereafter, the transformed strain was cultured in a Luria-Bertani (LB) solid medium to which the antibiotic ampicillin was added, and then the colonies obtained herein were cultured in a LB liquid medium in a 37° C. shaking incubator. Then, when the cell density reached about 0.2 absorbance at OD600, IPTG was added so that a final 0.5 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 24, it was confirmed that a Granzyme B protein having a size of about 35 kDa in the form to which TOM70, a linker and ubiquitin were fused was expressed. In this case, lane M in FIG. 24 shows a protein molecular weight marker, lane 1 shows the precipitate centrifuged after E. coli was crushed 4 hours after adding IPTG, and lane 2 shows the supernatant centrifuged after E. coli was crushed.

### Example 2.2.2. Preparation of a Plasmid, pET15b-UB-Granzyme B-TOM7

In order to prepare a Granzyme B protein in the form to which ubiquitin and TOM7 binding to the mitochondrial outer membrane were fused, the expression vector capable of expressing the Granzyme B protein in the form to which ubiquitin, Granzyme B, and TOM7 were fused in the order was prepared.

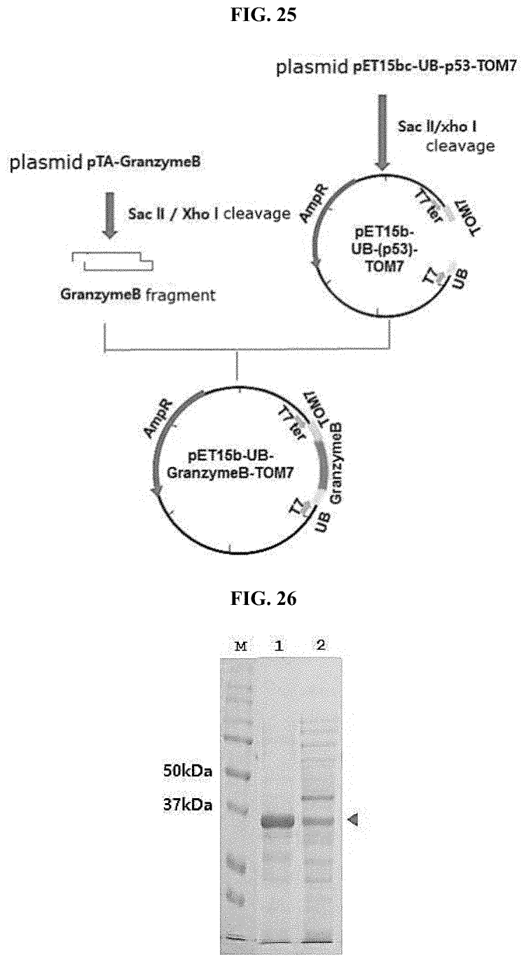

The plasmid pTA-Granzyme B gene obtained in Example 2.1. above was cleaved by the restriction enzymes SacII and XhoI, and and the DNA fragment of about 700 bp was obtained by electrophoresis on 2% agarose gel. Thereafter, it was inserted into a pET15b-UB-(p53)-TOM7 vector cleaved by the restriction enzymes SacII and XhoI using a T4DNA ligase to obtain the plasmid pET15b-UB-GranzymeB-TOM7(FIG. 25). Here, the UB-GranzymeB-TOM7 was represented by the base sequence of SEQ ID NO: 28.

E. coli BL21(DE3) strain was transformed using the plasmid pET15b-UB-Granzyme B-TOM7. Thereafter, the transformed strain was cultured in a Luria-Bertani (LB) solid medium to which the antibiotic ampicillin was added, and then the colonies obtained herein were cultured in a LB liquid medium under a condition of 37° C. Then, when the cell density reached about 0.2 absorbance at OD600, IPTG was added so that a final 0.5 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 26, it was confirmed that a Granzyme B protein having a size of about 35 kDa in the form to which ubiquitin and TOM70 were fused was expressed. In this case, lane M in FIG. 26 shows a protein molecular weight marker, lane 1 shows the precipitate centrifuged after E. coli was crushed 4 hours after adding IPTG, and lane 2 shows the supernatant centrifuged after E. coli was crushed.

### Example 2.3. Isolation and Purification of Recombinant TOM70-(GGGGS)3-UB-Granzyme B Protein Derived from E. coli

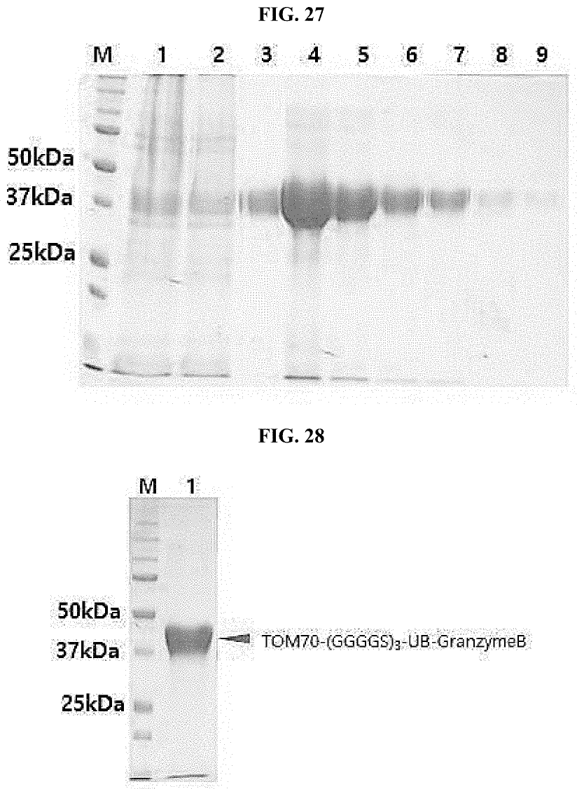

The TOM70-(GGGGS)3-UB-GranzymeB protein was isolated and purified in the same method as in Example 1.3.1. As a result, the TOM70-(GGGGS)3-UB-GranzymeB protein was eluted (FIG. 27). In this case, lane M in FIG. 27 shows a protein molecular weight marker, and lane 1 shows a nickel affinity chromatography loading sample. Lane 2 shows that it was not bound to a nickel affinity resin. Lanes 3 and 4 show the results of elution with 8M UREA/50 mM Na-phosphate/500 mM NaCl/50 mM Imidazole solution. Lanes 5 to 7 show the results of elution with 8M UREA/50 mM Na-phosphate/500 mM NaCl/100 mM Imidazole solution. Lanes 8 to 9 show the results of elution with 8M UREA/50 mM Na-phosphate/500 mM NaCl/250 mM Imidazole solution.

The protein amount of the recovered eluted solution was measured by protein quantification method and confirmed using SDS-PAGE. As shown in FIG. 28, after the confirmation was completed, the TOM70-(GGGGS)3-UB-Granzyme B protein was quenched with the liquid nitrogen and stored in a cryogenic freezer at −80° C. In this case, lane M in FIG. 28 shows a protein molecular weight marker, and lane 1 shows the TOM70-(GGGGS)3-UB-Granzyme B protein obtained after dialysis in PBS buffer solution.

### Example 3. Preparation of Fusion Protein Comprising RKIP

### Example 3.1. Amplification of RKIP Gene

In order to express the human RKIP (Raf Kinase Inhibitory Protein) gene into a recombinant protein, total RNA was extracted from human epithelial cells, and cDNA was synthesized therefrom. Human dermal fibroblast cells were cultured in 10% serum medium under a condition of 5% carbon dioxide and 37° C. (1×10⁶ cells). Thereafter, the RNA was obtained in the same method as in Example 1.1., and then it was used as a template for the polymerase chain reaction of the RKIP gene.

In order to obtain the gene of RKIP in which the signal peptide sequence was removed from human dermal fibroblast cells, T2RKIP primer encoding from the amino terminus proline and XRKIP(noT) primer encoding from the carboxyl terminus were synthesized, and then PCR was performed using the cDNA prepared above as a template. The sequence of each primer is as described in Table 9 below.

TABLE 9

| Primer | Sequence | SEQ ID NO. |
|---|---|---|
| T2RKIP | 5′-AAA AAA CCG CGG TGG Tcc ggt gga cct cag caa gtg gtc-3′ | SEQ ID NO: 29 |
| XRKIP(noT) | 5′-AAA AAA CTC GAG CTT CCC AGA CAG CTG CTC GTA CAG TTT | SEQ ID NO: 30 |
| | GG-3′ | |

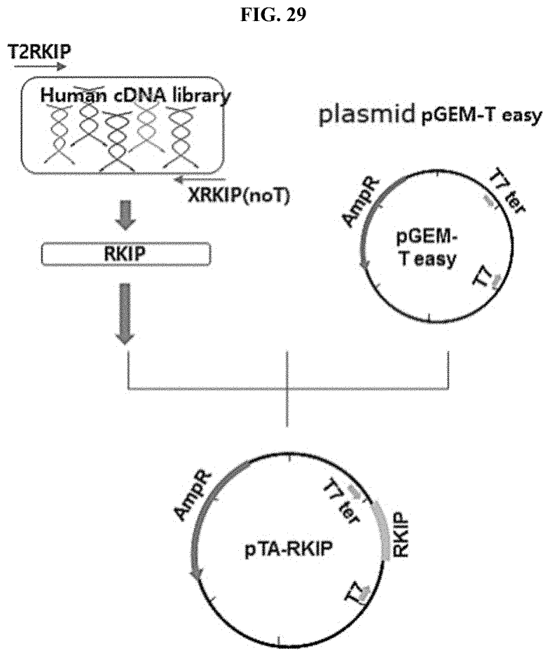

The cDNA prepared above was used as a template, and 0.2 pmol T2RKIP primer and 0.2 pmol XRKIP(noT) primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 40 cycles. After the reaction, the amplified DNA fragment of about 560 bp was isolated by electrophoresis on 1% agarose gel, and then inserted into a pGEM-T easy (Promega, USA) vector using a T4DNA ligase. As a result of sequencing the DNA thus obtained, it was confirmed that the cDNA encoding a human RKIP protein was obtained. The obtained RKIP gene was designated as pTA-RKIP (FIG. 29), and the base sequence of the RKIP gene was represented by the base sequence of SEQ ID NO: 31.

### Example 3.2. Preparation of an E. coli Expression Vector for RKIP Protein

### Example 3.2.1. Preparation of a Plasmid, pET11c-TOM70-(GGGGS)3-UB-RKIP

In order to prepare the RKIP protein in the form to which TOM70 binding to the mitochondrial outer membrane, a linker (GGGGSGGGGSGGGGS) and ubiquitin were fused, the expression vector capable of expressing RKIP in the form to which TOM70, a linker, and ubiquitin were fused was prepared.

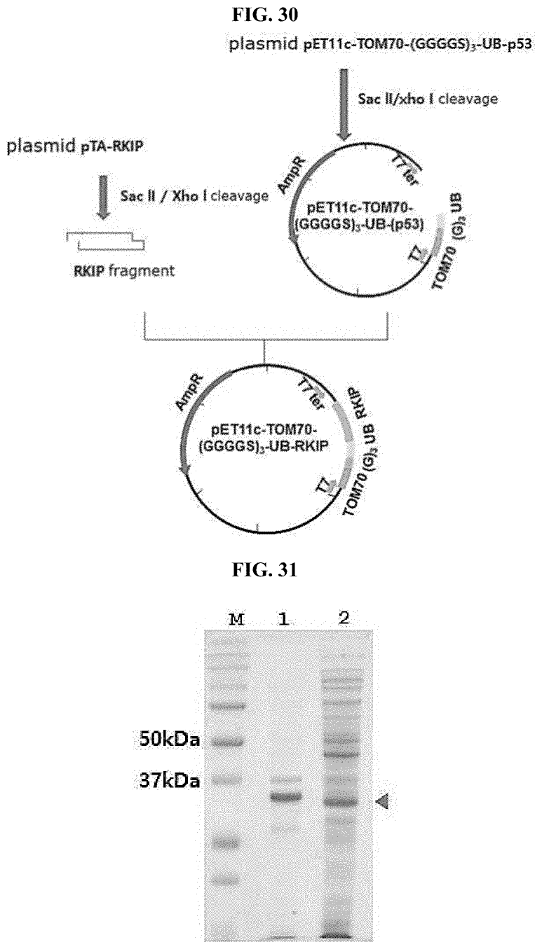

The plasmid pTA-RKIP gene obtained in Example 3.1. was cleaved by the restriction enzymes SacII and XhoI, and the DNA fragment of about 560 bp was obtained by electrophoresis on 2% agarose gel, and then it was inserted into a pET11c-TOM70-(GGGGS)3-UB-(p53) vector cleaved by the restriction enzymes SacII and XhoI using a T4DNA ligase to obtain the plasmid pET11-TOM70-(GGGGS)3-UB-RKIP (FIG. 30). Here, TOM70-(GGGGS)3-UB-RKIP was represented by the base sequence of SEQ ID NO: 32.

E. coli BL21(DE3) strain was transformed using the plasmid pET11c-TOM70-(GGGGS)3-UB-RKIP. Thereafter, the transformed strain was cultured in a Luria-Bertani (LB) solid medium to which the antibiotic ampicillin was added, and then the colonies obtained herein were cultured in a LB liquid medium in a 37° C. shaking incubator. Then, when the cell density reached about 0.2 absorbance at OD600, IPTG was added so that a final 0.5 mM concentration was made, and then the shaking culture was performed further for about 4 hours. A portion of E. coli cells was obtained by centrifugation, and then the cells were crushed, and then SDS-polyacrylamide electrophoresis was performed. As shown in FIG. 31, it was confirmed that a RKIP protein having a size of about 33 kDa in the form to which TOM70, a linker and ubiquitin were fused was expressed. In this case, lane M in FIG. 31 shows a protein molecular weight marker, lane 1 shows the precipitate centrifuged after E. coli was crushed 4 hours after adding IPTG, and lane 2 shows the supernatant centrifuged after E. coli was crushed.

### Example 3.3. Isolation and Purification of Recombinant TOM70-(GGGGS)3-UB-RKIP Protein Derived from E. coli

E. coli BL21(DE3) production strain expressing a recombinant TOM70-(GGGGS)3-UB-RKIP was inoculated into a LB liquid medium, and cultured under a condition of 37° C. When the absorbance reached 0.3 at OD600, it was put in a refrigerator to lower the temperature of the culture solution, and the temperature of the incubator was changed to 18° C., and then 0.5 mM IPTG was added, and the shaking culture was performed further for 1 day to express the TOM70-(GGGGS)3-UB-RKIP protein.

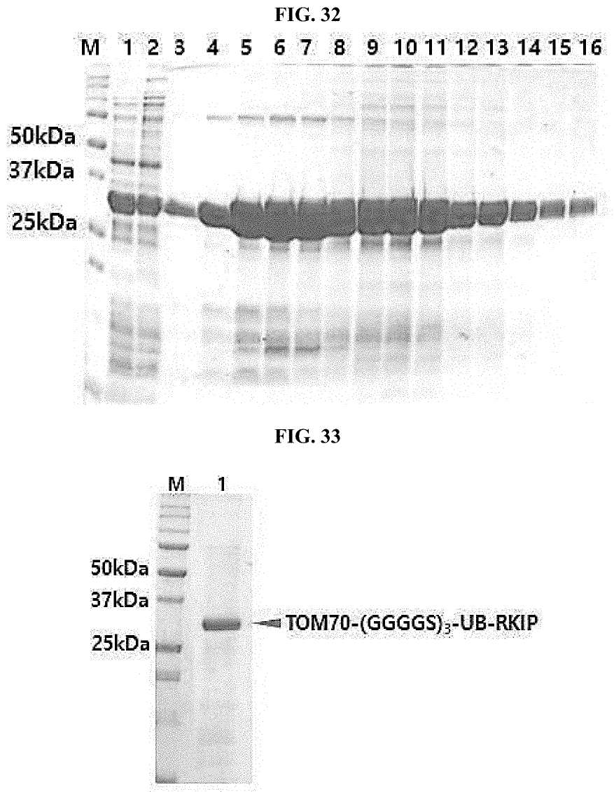

Then, the TOM70-(GGGGS)3-UB-RKIP protein was isolated and purified in the same method as in Example 1.3.1. As a result, the TOM70-(GGGGS)3-UB-RKIP protein was eluted (FIG. 32). In this case, lane M in FIG. 32 shows a protein molecular weight marker, and lane 1 shows a nickel affinity chromatography loading sample. Lane 2 shows that it was not bound to a nickel affinity resin. Lane 3 shows the results of elution with 50 mM Na-phosphate/500 mM NaCl/10 mM Imidazole. Lanes 4 to 6 show the results of elution with 50 mM Na-phosphate/500 mM NaCl/50 mM Imidazole. Lanes 7 to 8 show the results of elution with 50 mM Na-phosphate/500 mM NaCl/100 mM Imidazole. Lanes 9 to 10 show the results of elution with 50 mM Na-phosphate/500 mM NaCl/175 mM Imidazole. Lanes 11 to 13 show the results of elution with 50 mM Na-phosphate/500 mM NaCl/250 mM Imidazole. Lanes 14 to 16 show the results of elution with 50 mM Na-phosphate/500 mM NaCl/500 mM Imidazole. The protein amount of the recovered eluted solution was measured by protein quantification method and confirmed using SDS-PAGE. As shown in FIG. 33, after the confirmation was completed, the TOM70-(GGGGS)3-UB-RKIP protein was quenched with the liquid nitrogen and stored in a cryogenic freezer at −80° C. In this case, lane M in FIG. 33 shows a protein molecular weight marker, and lane 1 shows the TOM70-(GGGGS)3-UB-RKIP protein obtained after dialysis in PBS buffer solution.

### Example 4. Preparation of Fusion Protein Comprising PTEN

### Example 4.1. Amplification of PTEN Gene

In order to express the human PTEN (Phosphatase and Tensin homolog) into a recombinant protein, total RNA was extracted from human epithelial cells, and cDNA was synthesized therefrom. Fibroblast cells (human dermal fibroblast cells) were cultured in 10% serum medium under a condition of 5% carbon dioxide and 37° C. (1×10⁶ cells). Thereafter, the RNA was obtained in the same method as in Example 1.1., and then it was used as a template for the polymerase chain reaction of the PTEN gene.

In order to obtain the gene of PTEN in which the signal peptide sequence was removed from human dermal fibroblast cells, T2PTEN primer encoding from the amino terminus threonine and XPTEN(noT) primer encoding from the carboxyl terminus were synthesized, and then PCR was performed using the cDNA prepared above as a template. The sequence of each primer is as described in Table 10 below.

TABLE 10

| Primer | Sequence | SEQ ID NO. |
|---|---|---|
| T2PTEN | 5′-AAA AAA CCG CGG TGG Tac agc cat cat caa aga gat cgt tag-3′ | SEQ ID NO: 33 |
| XPTEN(noT) | 5′-AAA AAA CTC GAG GAC TTT TGT AAT TTG TGT ATG CTG-3′ | SEQ ID NO: 34 |

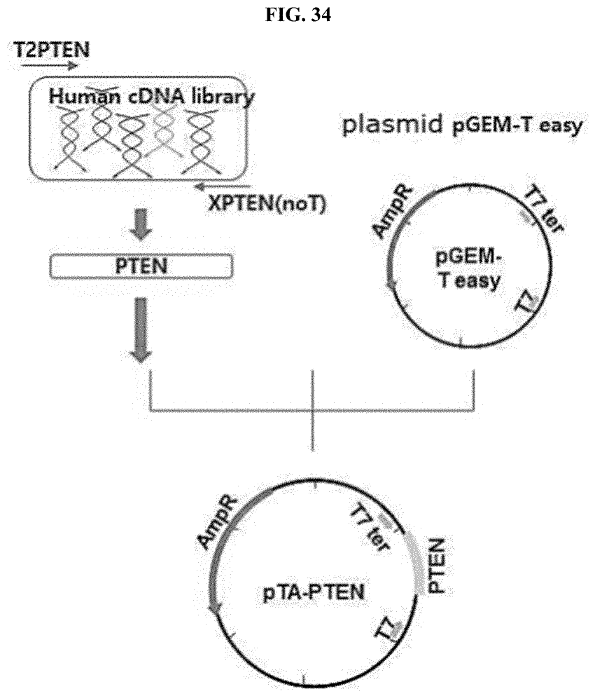

The cDNA prepared above was used as a template, and 0.2 pmol T2PTEN primer and 0.2 pmol XPTEN(noT) primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 40 cycles. After the reaction, the amplified DNA fragment of about 1,200 bp was isolated by electrophoresis on 1% agarose gel, and then inserted into a pGEM-T easy (Promega, USA) vector using a T4DNA ligase. As a result of sequencing the DNA thus obtained, it was confirmed that the cDNA encoding a human RKIP protein was obtained. The obtained PTEN gene was designated as pTA-PTEN (FIG. 34), and the base sequence of the PTEN was represented by the base sequence of SEQ ID NO: 35.

### Example 4.2. Preparation of an E. coli Expression Vector for PTEN Protein

### Example 4.2.1. Preparation of a Plasmid, pET11c-TOM70-(GGGGS)3-UB-PTEN

In order to prepare a PTEN protein in the form to which TOM70 binding to the mitochondrial outer membrane, a linker (GGGGSGGGGSGGGGS) and ubiquitin were fused, the expression vector capable of expressing the PTEN gene in the form to which TOM70, the linker and ubiquitin were fused was prepared.

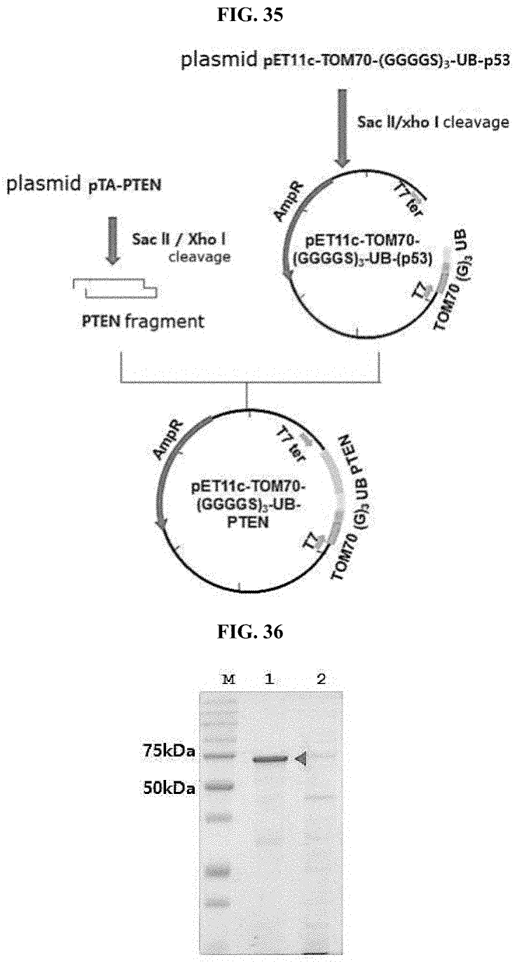

The plasmid pTA-PTEN gene obtained in Example 4.1. above was cleaved by the restriction enzymes SacII and XhoI, and and the DNA fragment of about 1,200 bp was obtained by electrophoresis on 2% agarose gel. Thereafter, it was inserted into a pET11c-TOM70-(GGGGS)3-UB-(p53) vector cleaved by the restriction enzymes SacII and XhoI using a T4DNA ligase to obtain the plasmid pET11c-TOM70-(GGGGS)3-UB-PTEN (FIG. 35). Here, TOM70-(GGGGS)3-UB-PTEN was represented by the base sequence of SEQ ID NO: 36.

E. coli BL21(DE3) strain was transformed using the plasmid pET11c-TOM70-(GGGGS)3-UB-PTEN. Thereafter, the transformed strain was cultured in a Luria-Bertani (LB) solid medium to which the antibiotic ampicillin was added, and then the colonies obtained herein were cultured in a LB liquid medium under the condition of 37° C. Then, when the cell density reached about 0.2 absorbance at OD600, IPTG was added so that a final 0.5 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 36, it was confirmed that a PTEN protein having a size of about 73 kDa in the form to which TOM70, a linker and ubiquitin were fused was expressed. In this case, lane M in FIG. 36 shows a protein molecular weight marker, lane 1 shows the precipitate centrifuged after E. coli was crushed 4 hours after adding IPTG, and lane 2 shows the supernatant centrifuged after E. coli was crushed.

### Example 4.3 Isolation and Purification of Recombinant TOM70-(GGGGS)3-UB-PTEN Protein Derived from E. coli

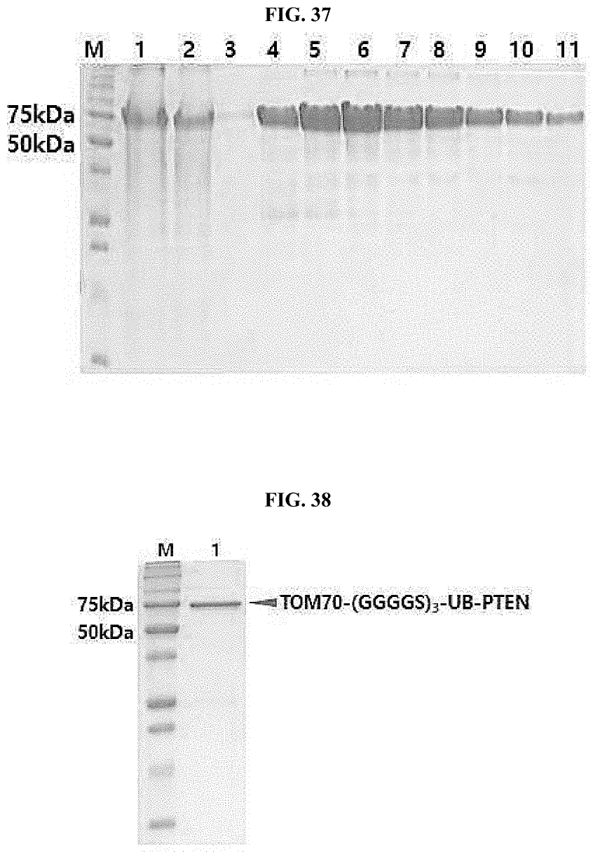

The TOM70-(GGGGS)3-UB-PTEN protein was isolated and purified in the same method as in Example 1.3.1. As a result, the TOM70-(GGGGS)3-UB-PTEN protein was eluted (FIG. 37). In this case, lane M in FIG. 37 shows a protein molecular weight marker, and lane 1 shows a nickel affinity chromatography loading sample. Lane 2 shows that it was not bound to a nickel affinity resin. Lane 3 shows the results of elution with 8M UREA/50 mM Na-phosphate/500 mM NaCl/10 mM Imidazole solution. Lane 4 shows the results of elution with 8M UREA/50 mM Na-phosphate/500 mM NaCl/50 mM Imidazole solution. Lanes 5 to 8 show the results of elution with 8M UREA/50 mM Na-phosphate/500 mM NaCl/100 mM Imidazole solution. Lanes 9 to 10 show the results of elution with 8M UREA/50 mM Na-phosphate/500 mM NaCl/250 mM Imidazole solution. Lane 11 shows the results of elution with 8M UREA/50 mM Na-phosphate/500 mM NaCl/500 mM Imidazole solution.

The protein amount of the recovered eluted solution was measured by protein quantification method and confirmed using SDS-PAGE. As shown in FIG. 38, after the confirmation was completed, the TOM70-(GGGGS)3-UB-PTEN protein was quenched with the liquid nitrogen and stored in a cryogenic freezer at −80° C. In this case, lane M in FIG. 38 shows a protein molecular weight marker, and lane 1 shows the TOM70-(GGGGS)3-UB-PTEN protein obtained after dialysis in PBS buffer solution.

### Example 5. Preparation of Fusion Protein Comprising Mitochondrial Outer Membrane Protein, Ubiquitin and GFP

### Example 5.1. Isolation and Purification of Recombinant UB-GFP-TOM7 Protein Derived from E. coli

E. coli BL21(DE3) production strain expressing a UB-GFP-TOM7 protein in the mature form to which ubiquitin was fused was inoculated into a LB liquid medium, and cultured under a condition of 37° C. When the absorbance reached 0.3 at OD600, it was put in a refrigerator to lower the temperature of the culture solution, and the temperature of the incubator was changed to 18° C., and then 0.5 mM IPTG was added, and the shaking culture was performed further for 1 day to express the GFP-TOM7 protein in the mature form to which ubiquitin was fused.

After the culture was completed, the cells were recovered using centrifugation, and the recovered cells were washed once using PBS, and then the cells were suspended using 50 mM sodium phosphate, 500 mM NaCl, 10 mM imidazole, pH 8.0 solution, and the suspended cells were subjected to a crushing process using a sonicator. The crushed cells were centrifuged using a high speed centrifuge, and then the supernatant was recovered, and the recovered supernatant was filtered using a 0.45 μm filter, and then loaded on a pre-packed nickel chromatography column to perform primary purification.

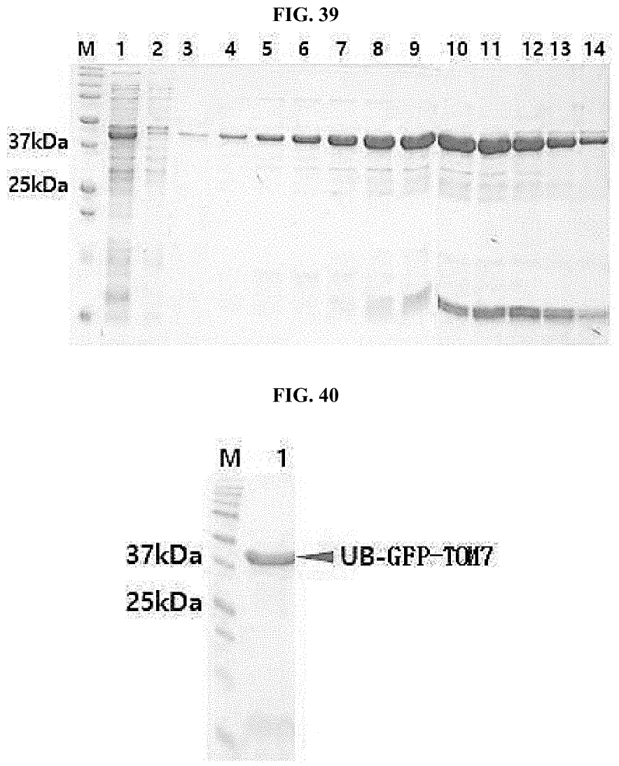

The crushing solution comprising the UB-GFP-TOM7 protein in the mature form to which ubiquitin was fused was loaded, and then until the impurities unbound were not detected, a washing solution was flowed using 50 mM sodium phosphate, 500 mM NaCl, 20 mM imidazole, pH 8.0 solution, and the protein was eluted according to the concentration gradient using 50 mM sodium phosphate, 500 mM NaCl, 500 mM imidazole, pH 8.0 solution (FIG. 39). In this case, lane M in FIG. 39 shows a protein molecular weight marker, and lane 1 shows a nickel affinity chromatography loading sample. Lane 2 shows that it was not bound to a nickel affinity resin. Lane 3 shows the results of elution with 50 mM Na-phosphate/500 mM NaCl/20 mM Imidazole. Lane 4 shows the results of elution with 50 mM Na-phosphate/500 mM NaCl/55 mM Imidazole. Lane 5 shows the results of elution with 50 mM Na-phosphate/500 mM NaCl/60 mM Imidazole. Lane 6 shows the results of elution with 50 mM Na-phosphate/500 mM NaCl/65 mM Imidazole. Lane 7 shows the results of elution with 50 mM Na-phosphate/500 mM NaCl/70 mM Imidazole. Lane 8 shows the results of elution with 50 mM Na-phosphate/500 mM NaCl/75 mM Imidazole. Lane 9 shows the results of elution with 50 mM Na-phosphate/500 mM NaCl/80 mM Imidazole. Lane 10 shows the results of elution with 50 mM Na-phosphate/500 mM NaCl/85 mM Imidazole. Lane 11 shows the results of elution with 50 mM Na-phosphate/500 mM NaCl/90 mM Imidazole. Lane 12 shows the results of elution with 50 mM Na-phosphate/500 mM NaCl/95 mM Imidazole. Lane 13 shows the results of elution with 50 mM Na-phosphate/500 mM NaCl/100 mM Imidazole. Lane 14 shows the results of elution with 50 mM Na-phosphate/500 mM NaCl/105 mM Imidazole.

In order to remove imidazole in the eluted solution, dialysis was performed using the principle of osmotic pressure in a 50 mM sodium phosphate, 500 mM NaCl, pH 8.0 solution (FIG. 40). The final UB-GFP-TOM7 protein that was identified was quenched with the liquid nitrogen and stored in a cryogenic freezer at −80° C. In this case, lane M in FIG. 40 shows a protein molecular weight marker, and lane 1 shows a protein obtained after dialysis was performed in a 50 mM Na-phosphate/500 mM NaCl solution after mixing a fusion protein fraction.

### Example 5.2. Isolation and Purification of Recombinant TOM70-(GGGGS)3-UB-GFP Protein Derived from E. coli

E. coli BL21(DE3) production strain expressing a recombinant protein TOM70-(GGGGS)3-UB-GFP was inoculated into a LB liquid medium, and cultured under a condition of 37° C. When the absorbance reached 0.3 at OD600, it was put in a refrigerator to lower the temperature of the culture solution, and the temperature of the incubator was changed to 18° C., and then 0.5 mM IPTG was added, and the shaking culture was performed further for 1 day to express the recombinant protein TOM70-(GGGGS)3-UB-GFP.

After the culture was completed, the cells were recovered using centrifugation, and the recovered cells were washed once using PBS, and then the cells were suspended using 50 mM sodium phosphate, 500 mM NaCl, 10 mM imidazole, pH 8.0 solution, and the suspended cells were subjected to a crushing process using a sonicator. The crushed cells were centrifuged using a high speed centrifuge, and then the supernatant was recovered, and the recovered supernatant was filtered using a 0.45 μm filter, and then loaded on a pre-packed nickel chromatography column to perform primary purification.

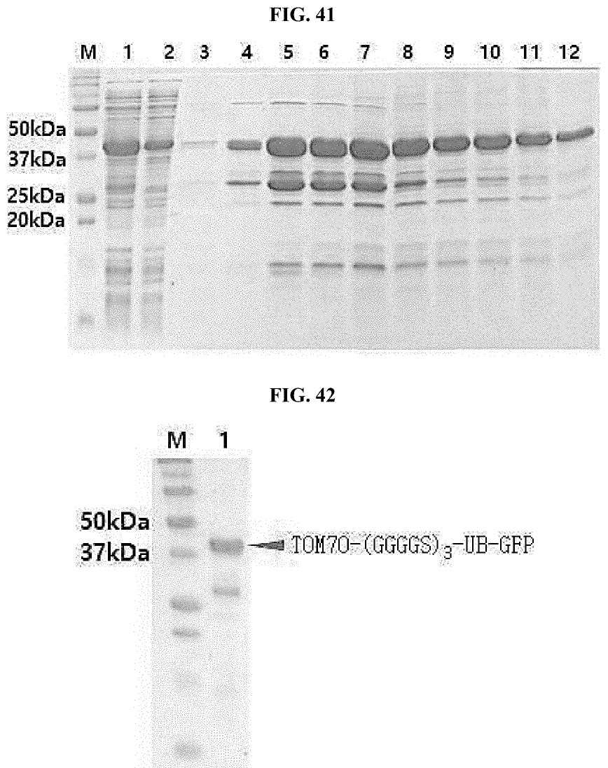

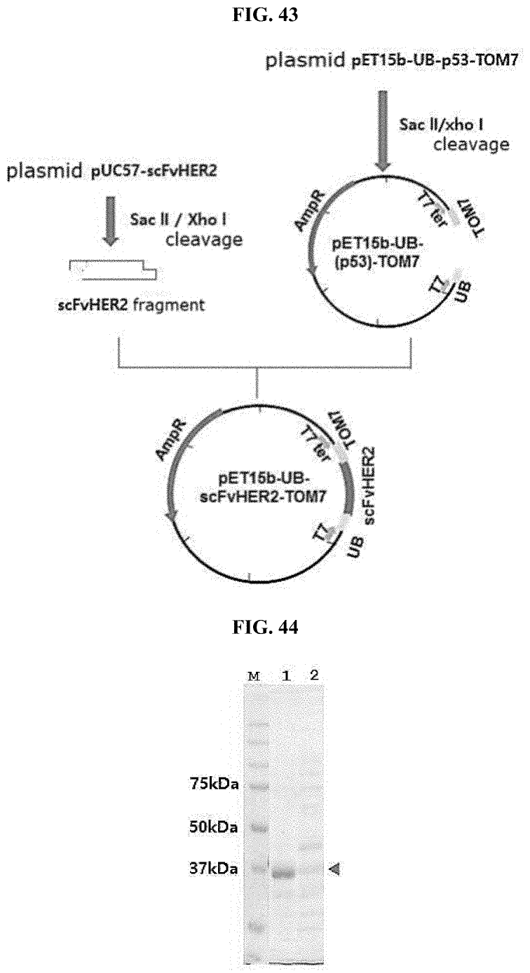

The crushing solution comprising the recombinant protein the TOM70-(GGGGS)3-UB-GFP was loaded on the column containing nikel resins, and then until the impurities unbound were not detected, a washing solution was flowed using 50 mM sodium phosphate, 500 mM NaCl, 20 mM imidazole, pH 8.0 solution. Then, the protein was eluted using 50 mM sodium phosphate, 500 mM NaCl, 500 mM imidazole, pH 8.0 solution, while changing the concentration of imidazole to 50 mM, 100 mM, 250 mM, 500 mM (FIG. 41). In this case, lane M in FIG. 41 shows a protein molecular weight marker, and lane 1 shows a nickel affinity chromatography loading sample. Lane 2 shows that it was not bound to a nickel affinity resin. Lane 3 shows the results of elution with 50 mM Na-phosphate/500 mM NaCl/20 mM Imidazole. Lane 4 shows the results of elution with 50 mM Na-phosphate/500 mM NaCl/50 mM Imidazole. Lanes 5 to 8 show the results of elution with 50 mM Na-phosphate/500 mM NaCl/100 mM Imidazole. Lanes 9 to 11 show the results of elution with 50 mM Na-phosphate/500 mM NaCl/250 mM Imidazole. Lane 12 shows the results of elution with 50 mM Na-phosphate/500 mM NaCl/500 mM Imidazole.

The eluted solution recovered from the nickel chromatography was solution-exchanged with PBS buffer solution using the principle of osmotic pressure. After the solution exchange was completed, the final protein TOM70-(GGGGS)3-UB-GFP that was recovered was identified using protein quantification and SDS-PAGE. As shown in FIG. 42, after the identification was completed, the TOM70-(GGGGS)3-UB-GFP protein was quenched with the liquid nitrogen and stored in a cryogenic freezer at −80° C. In this case, lane M in FIG. 42 shows a protein molecular weight marker, and lane 1 shows TOM70-(GGGGS)3-UB-GFP protein obtained after dialysis was performed in a PBS buffer solution.

### II. Preparation of Fusion Protein Comprising Mitochondrial Outer Membrane Targeting Protein and Target Targeting Protein

### Example 6. Preparation of Fusion Protein Comprising scFvHER2

### Example 6.1. Synthesis of scFvHER2 Gene

In order to express the human scFvHER2 into a recombinant protein, the scFvHER2 gene obtained by requesting gene synthesis from Bionics Co., Ltd. was designated as pUC57-scFvHER2, and the base sequence of scFvHER2 was the same as the base sequence of SEQ ID NO: 37.

### Example 6.2. Preparation of scFvHER2 Protein Expression Vector

### Example 6.2.1. pET15b-UB-scFvHER2-TOM7

In order to prepare a scFvHER2 protein in the form to which ubiquitin and TOM7 binding to the mitochondrial outer membrane were fused, the expression vector capable of expressing the scFvHER2 gene in the form to which ubiquitin and TOM7 were fused was prepared.

The plasmid pUC57-scFvHER2 gene obtained in Example 6.1. was cleaved by the restriction enzymes SacII and XhoI, and the DNA fragment of about 750 bp was obtained by electrophoresis on 2% agarose gel, and then it was inserted into a pET15b-UB-(p53)-TOM7 vector cleaved by the restriction enzymes SacII and XhoI using a T4DNA ligase to obtain the plasmid pET15b-UB-scFvHER2-TOM7 (FIG. 39). In this case, UB-scFvHER2-TOM7 was represented by the base sequence of SEQ ID NO: 38.

E. coli BL21(DE3) strain was transformed using the plasmid pET15b-UB-scFvHER2-TOM7. Thereafter, the transformed strain was cultured in a Luria-Bertani (LB) solid medium to which the antibiotic ampicillin was added, and then the colonies obtained herein were cultured in a LB liquid medium under the condition of 37° C. Then, when the cell density reached about 0.2 absorbance at OD600, IPTG was added so that a final 1 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 44, it was confirmed that a scFvHER2 protein having a size of about 35 kDa in the form to which ubiquitin and TOM7 were fused was expressed. In this case, lane M in FIG. 44 shows a protein molecular weight marker, lane 1 shows the precipitate centrifuged after E. coli was crushed 4 hours after adding IPTG, and lane 2 shows the supernatant centrifuged after E. coli was crushed.

### Example 6.2.2. Preparation of pCMV-scFvHER2-TOM7-myc/His

In order to prepare a scFvHER2 protein in the form to which TOM7 binding to the mitochondrial outer membrane was fused, an expression vector for animal cells capable of expressing scFvHER2 in the form to which TOM7 was fused was prepared. In order to obtain the TOM7 and scFvHER2 genes, RscFvHER2 primer and XTOM7(noT) primer were prepared. The sequence of each primer is as described in Table 11 below.

TABLE 11

| Primer | Sequence | SEQ ID NO. |
|---|---|---|
| RscFvHER2 | 5′-AAA AAA GAA TTC ATG GAA GTG CAA CTT GTT GAG AGT GG- | SEQ ID NO: 39 |
| | 3′ | |
| XTOM7(noT) | 5′-AAA AAA CTC GAG TCC CCA AAG TAG GCT CAA AAC AG-3′ | SEQ ID NO: 40 |

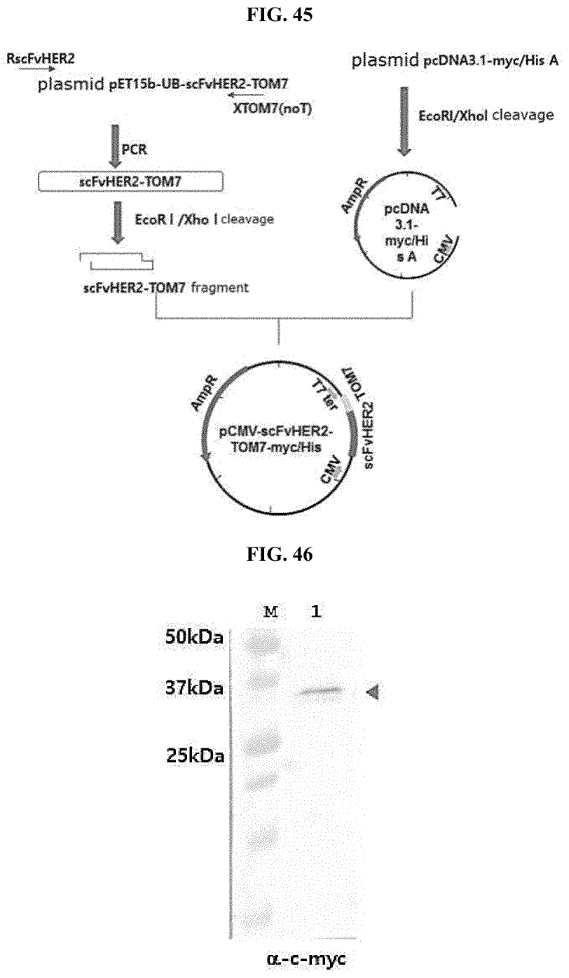

The plasmid pET15b-UB-scFvHER2-TOM7 obtained in Example 6.2.1. was used as a template, and 0.2 pmol primer (RscFvHER2) and 0.2 pmol primer (XTOM7(noT)) were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 25 cycles to obtain a gene scFvHER2-TOM7. The amplified scFvHER2-TOM7 gene was cleaved by the restriction enzymes EcoRI and XhoI, and the DNA fragments of about 850 bp, respectively, were obtained by electrophoresis on 1% agarose gel, and then inserted into a pcDNA3.1-myc/His A vector cleaved by the restriction enzymes EcoRI and XhoI using a T4DNA ligase to obtain the plasmid pCMV-scFvHER2-TOM7-myc/His (FIG. 45).

In this case, scFvHER2-TOM7-myc/His was represented by the base sequence of SEQ ID NO: 41. It was transfected into an animal cell CHO using the plasmid pCMV-scFvHER2-TOM7-myc/His, and the cells was crushed, and then SDS-polyacrylamide electrophoresis was performed, and it was shown by Western blot using an anti-c-myc antibody. As shown in FIG. 46, it was confirmed that a scFvHER2 protein having a size of about 35 kDa in the form to which TOM7 was fused was expressed. In this case, lane M in FIG. 46 shows a protein molecular weight marker, and lane 1 shows that it was transfected into an animal cell CHO, and the cells was crushed, and then SDS-polyacrylamide electrophoresis was performed, and then it was confirmed by Western blot using an anti-c-myc antibody.

### Example 6.3. Isolation and Purification of Recombinant UB-ScFvHER2-TOM7 Protein Derived from E. coli

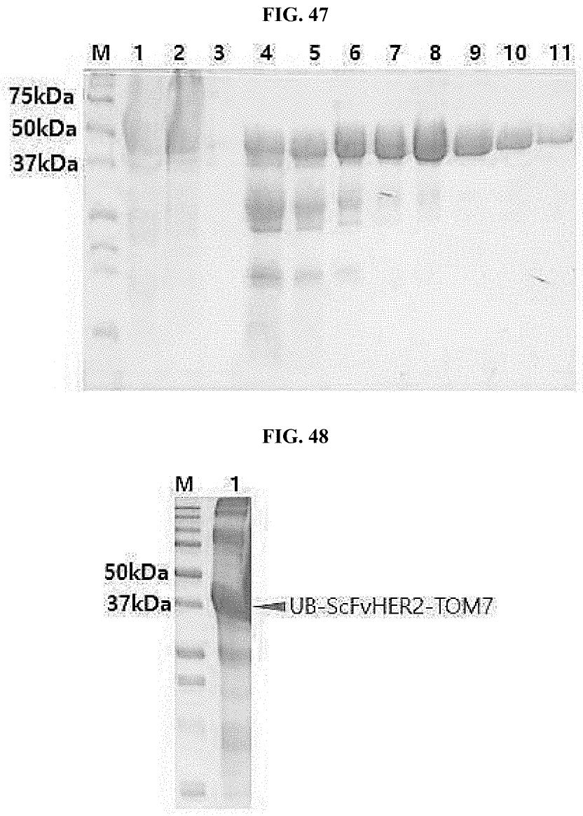

The UB-ScFvHER2-TOM7 protein was isolated and purified in the same method as in Example 1.3.1. As a result, the UB-ScFvHER2-TOM7 protein was eluted (FIG. 47). In this case, lane M in FIG. 47 shows a protein molecular weight marker, and lane 1 shows a nickel affinity chromatography loading sample. Lane 2 shows that it was not bound to a nickel affinity resin. Lane 3 shows the results of elution with 8M UREA/50 mM Na-phosphate/500 mM NaCl/10 mM Imidazole. Lanes 4 to 5 show the results of elution with 8M UREA/50 mM Na-phosphate/500 mM NaCl/50 mM Imidazole. Lanes 6 to 8 show the results of elution with 8M UREA/50 mM Na-phosphate/500 mM NaCl/100 mM Imidazole. Lanes 9 to 10 show the results of elution with 8M UREA/50 mM Na-phosphate/500 mM NaCl/250 mM Imidazole. Lane 11 shows the results of elution with 8M UREA/50 mM Na-phosphate/500 mM NaCl/500 mM Imidazole.

The protein amount of the recovered eluted solution was measured by protein quantification method and confirmed using SDS-PAGE. As shown in FIG. 48, after the confirmation was completed, the UB-ScFvHER2-TOM7 protein was quenched with the liquid nitrogen and stored in a cryogenic freezer at −80° C. In this case, lane M in FIG. 48 shows a protein molecular weight marker, and lane 1 shows the UB-ScFvHER2-TOM7 protein obtained after dialysis in PBS buffer solution.

### Example 7. Preparation of Fusion Protein Comprising scFvMEL

### Example 7.1. Synthesis of scFvMEL Gene

In order to express the human scFvMEL into a recombinant protein as an antibody fragment against melanoma, the scFvMEL gene obtained by requesting gene synthesis from Bionics Co., Ltd. was designated as pUC57-scFvMEL, and the base sequence of scFvMEL was the same as the base sequence of SEQ ID NO: 42.

### Example 7.2. Preparation of scFvMEL Protein Expression Vector

### Example 7.2.1. Preparation of pET15b-UB-scFvMEL-TOM7

In order to prepare a scFvMEL protein in the form to which ubiquitin and TOM7 binding to the mitochondrial outer membrane were fused, an expression vector capable of expressing scFvMEL in the form to which ubiquitin and TOM7 were fused was prepared.

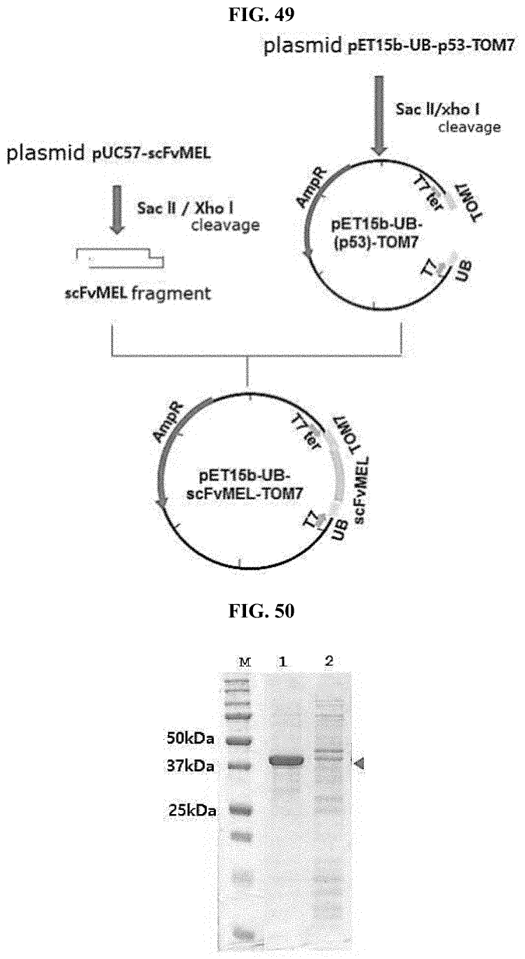

The plasmid pUC57-scFvMEL gene obtained in Example 7.1. was cleaved by the restriction enzymes SacII and XhoI, and the DNA fragment of about 750 bp was obtained by electrophoresis on 2% agarose gel, and then inserted into a pET15b-UB-(p53)-TOM7 vector cleaved by the restriction enzymes SacII and XhoI using a T4DNA ligase to obtain the plasmid pET15b-UB-scFvMEL-TOM7 (FIG. 49). In this case, UB-scFvMEL-TOM7 was represented by the base sequence of SEQ ID NO: 43.

E. coli BL21(DE3) strain was transformed using the plasmid pET15b-UB-scFvMEL-TOM7. Thereafter, the transformed strain was cultured in a Luria-Bertani (LB) solid medium to which the antibiotic ampicillin was added, and then the colonies obtained herein were cultured in a LB liquid medium in a 37° C. shaking incubator. Thereafter, when the cell density reached about 0.2 absorbance at OD600, IPTG was added so that a final 1 mM concentration was made, and then the shaking culture was performed further for about 4 hours.

A portion of E. coli cells was obtained by centrifugation, and then the cells were crushed, and then SDS-polyacrylamide electrophoresis was performed. As shown in FIG. 50, it was confirmed that a scFvMEL protein having a size of about 35 kDa in the form to which ubiquitin and TOM7 were fused was expressed. In this case, lane M in FIG. 50 shows a protein molecular weight marker, lane 1 shows the precipitate centrifuged after E. coli was crushed 4 hours after adding IPTG, and lane 2 shows the supernatant centrifuged after E. coli was crushed.

### Example 7.2.2. Preparation of pCMV-scFvMEL-TOM7-myc/His

In order to prepare a scFvMEL protein in the form to which TOM7 binding to the mitochondrial outer membrane was fused, an expression vector for animal cells capable of expressing scFvMEL in the form to which TOM7 was fused was prepared. In order to obtain the TOM7 and scFvMEL genes, a primer (RscFvMEL) was prepared. The sequence of each primer is as described in Table 12 below.

TABLE 12

| Primer | Sequence | SEQ ID NO. |
|---|---|---|
| RscFvMEL | 5′-AAA AAA GAA TTC ATG AAA ACA AGT AAC CCA GGA GTG-3′ | SEQ ID NO: 44 |

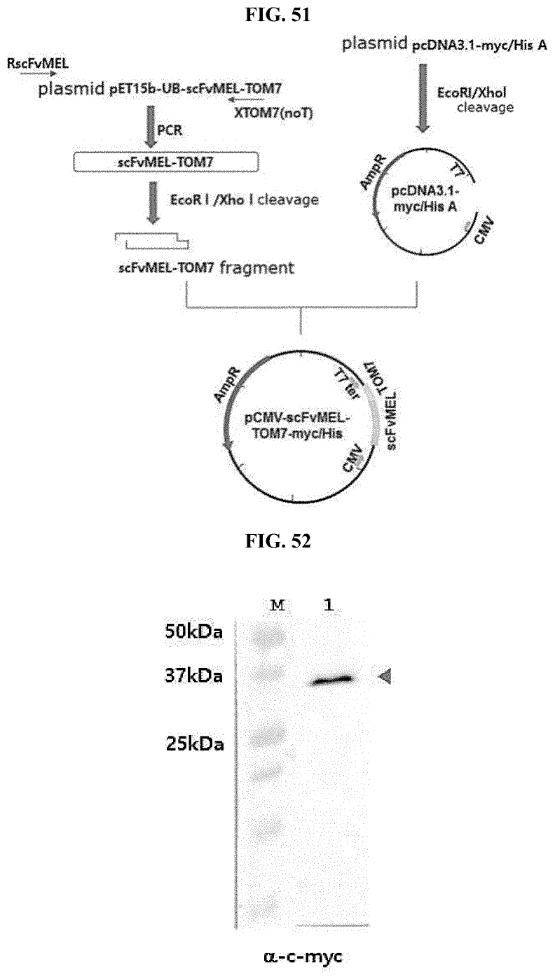

The plasmid pET15b-UB-scFvMEL-TOM7 obtained in Example 6.2.1. was used as a template, and 0.2 pmol RscFvMEL primer and 0.2 pmol XTOM7(noT) primer were mixed with dNTP 0.2 nM, 1× AccuPrime Taq DNA polymerase reaction buffer solution (Invitrogen, USA) and 1 unit of AccuPrime Taq DNA polymerase. Thereafter, in a polymerase chain reaction apparatus, amplification reactions of 95° C. for 40 seconds, 58° C. for 30 seconds, 72° C. for 1 minute were performed at 25 cycles to obtain scFvMEL-TOM7. The amplified scFvMEL-TOM7 gene was cleaved by the restriction enzymes EcoRI and XhoI, and the DNA fragment of about 850 bp was obtained by electrophoresis on 1% agarose gel. Then, it was inserted into a pcDNA3.1-myc/His A vector cleaved by the restriction enzymes EcoRI and XhoI using a T4DNA ligase to obtain the plasmid pCMV-scFvMEL-TOM7-myc/His (FIG. 51). Here, scFvMEL-TOM7-myc/His was represented by the base sequence of SEQ ID NO: 45.

It was transfected into an animal cell CHO using the plasmid pCMV-scFvMEL-TOM7-myc/His, and the cells was crushed, and then SDS-polyacrylamide electrophoresis was performed, and it was shown by Western blot using an anti-c-myc antibody. As shown in FIG. 52, it was confirmed that a scFvMEL protein having a size of about 35 kDa in the form to which TOM7 was fused was expressed. In this case, lane M in FIG. 52 shows a protein molecular weight marker, and lane 1 shows that it was transfected into an animal cell CHO, and the cells was crushed, and then SDS-polyacrylamide electrophoresis was performed, and then it was confirmed by Western blot using an anti-c-myc antibody.

### Example 8. Preparation of Fusion Protein Comprising scFvPD-L1

### Example 8.1. Synthesis of scFvPD-L1 Gene

In order to express the human scFvPD-L1 into a recombinant protein, the scFvPD-L1 gene obtained by requesting gene synthesis from Bionics Co., Ltd. was designated as pUC57-scFvPD-L1, whose base sequence was the same as the base sequence of SEQ ID NO: 46.

### Example 8.2. Preparation of scFvPD-L1 Protein Expression Vector

### Example 8.2.1. Preparation of pCMV-scFvPD-L1-TOM7-Myc/his

In order to prepare a scFvPD-L1 protein in the form to which TOM7 binding to the mitochondrial outer membrane was fused, an expression vector for animal cells capable of expressing scFvPD-L1 in the form to which ubiquitin and TOM7 was fused was prepared. The plasmid pUC57-scFvPD-L1 was cleaved by the restriction enzymes EcoRI and

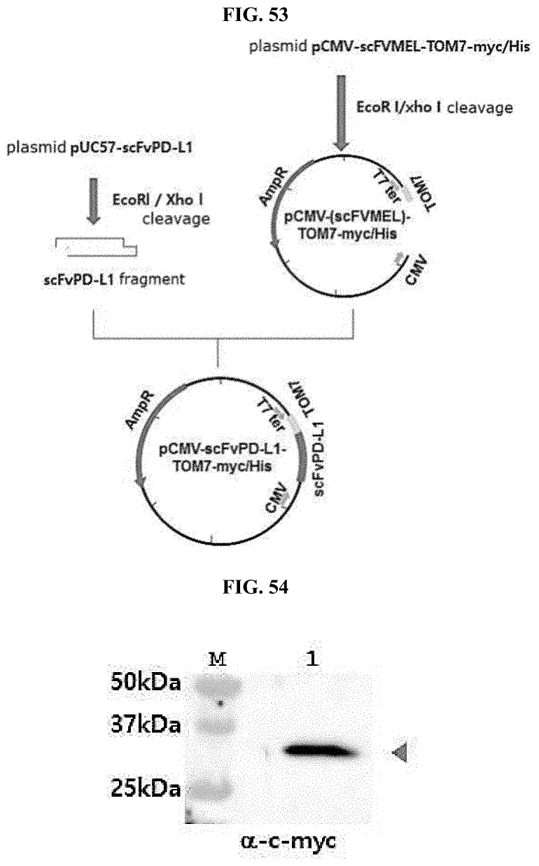

XhoI, and the DNA fragment of about 760 bp was obtained by electrophoresis on 1% agarose gel. Then, it was inserted into a pCMV-(scFvMEL)-TOM7-myc/His vector cleaved by the restriction enzymes EcoRI and XhoI using a T4DNA ligase to obtain the plasmid pCMV-scFvPD-L1-TOM7-myc/His (FIG. 53). In this case, scFvPD-L1-TOM7-myc/His was represented by the base sequence of SEQ ID NO: 47.

It was transfected into an animal cell CHO using the plasmid pCMV-scFvPD-L1-TOM7-myc/His, and the cells was crushed, and then SDS-polyacrylamide electrophoresis was performed, and it was shown by Western blot using an anti-c-myc antibody. As shown in FIG. 54, it was confirmed that a scFvPD-L1 protein having a size of about 35 kDa in the form to which TOM7 was fused was expressed. In this case, lane M in FIG. 54 shows a protein molecular weight marker, and lane 1 shows that it was transfected into an animal cell CHO, and the cells was crushed, and then SDS-polyacrylamide electrophoresis was performed, and then it was confirmed by Western blot using an anti-c-myc antibody.

### III. Preparation of Modified Mitochondria to which Fusion Protein was Bound

### Example 9. Preparation of Modified Mitochondria

The following experiment was conducted to confirm whether the fluorescent protein fused with the mitochondrial outer membrane binding site binds to the outer membrane of the mitochondria. First, the mitochondria were isolated from mesenchymal stem cells derived from umbilical cord (UC-MSCs) by centrifugation method. Thereafter, they were stained with MitoTracker CMXRos Red. They were mixed with the recombinant protein TOM70-(GGGGS)3-UB-GFP purified from E. coli in the above and incubated at ambient temperature for about 30 minutes.

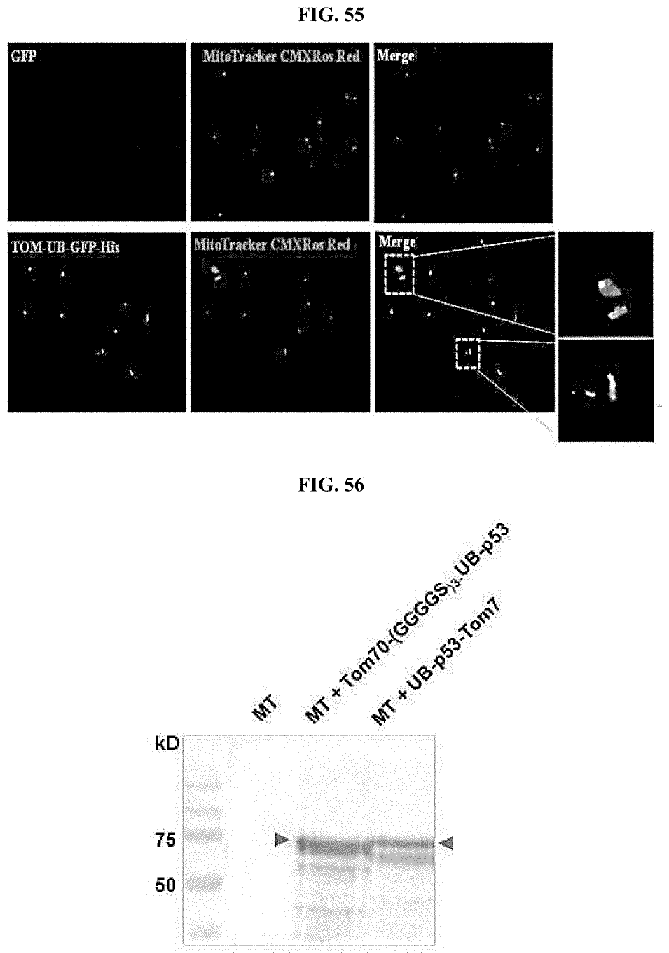

Thereafter, the unreacted protein was removed by centrifugation and washed twice with PBS buffer solution. Thereafter, the fluorescent protein in the form bound to the mitochondria was observed using a fluorescence microscope. As a control group, the purified GFP protein that does not comprise a mitochondrial outer membrane binding site was used. As a result, it was confirmed that the fluorescent protein fused with the mitochondrial outer membrane binding site (TOM70-(GGGGS)3-UB-GFP) was located in the same place as the mitochondria of mesenchymal stem cells derived from umbilical cord (UC-MSC) (FIG. 55a, FIG. 55b).

### Example 10. Confirmation of Ability of Recombinant Protein p53 to Bind to Foreign Mitochondrial Outer Membrane

The mitochondria that had been isolated from mesenchymal stem cells derived from umbilical cord using centrifugation method were mixed with the purified recombinant protein TOM70-(GGGGS)3-UB-p53 or UB-p53-TOM7, and were allowed to be bound at a ratio of 1:1 under a reaction condition of at 4° C. for 1 hour. As a control group, the mitochondria that were not mixed with the protein were used. The binding ability between mitochondria and p53 was confirmed through a Western blot experiment method (FIG. 56).

First, the mitochondria and the p53 protein were bound, and then centrifugation was performed at 13,000 rpm for 10 minutes to obtain the mitochondria or the mitochondria to which p53 was bound in the form of a precipitate. The protein that was not bound to the mitochondria was removed through a PBS washing process twice, and the washed precipitate was subjected to protein electrophoresis (SDS-PAGE) and then Western Blot. Rabbit anti-p53 antibody was used as a primary antibody, and anti-rabbit IgG HRP was used as a secondary antibody. The band was confirmed at the same position as a size of 60 kDa, which is a molecular weight expected in the experimental group for mitochondria that did bind to TOM70-(GGGGS)3-UB-p53 or UB-p53-TOM7, compared to the control group for mitochondria alone that did not bind to the protein (FIG. 56).

### IV. Confirmation of Activity of Modified Mitochondria to which Active Protein was Bound

### Example 11. Isolation and Intracellular Injection of Foreign Mitochondria

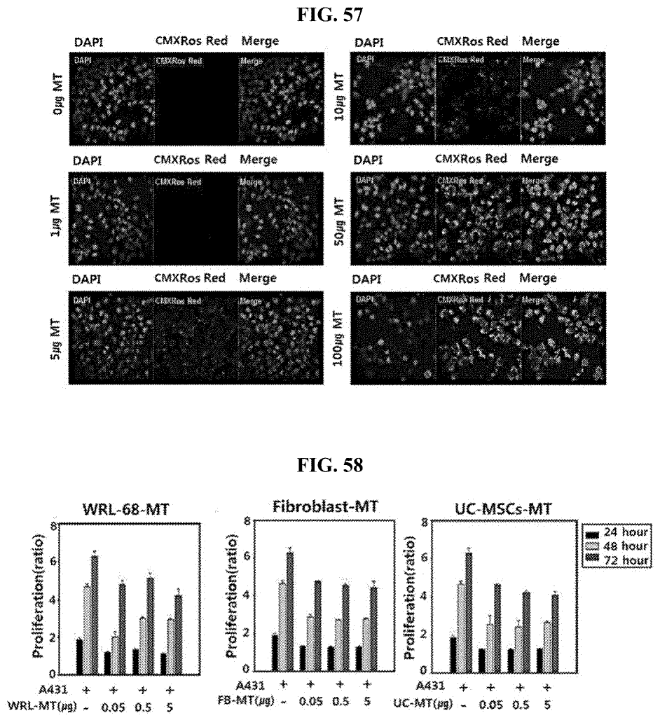

The mitochondria were isolated from mesenchymal stem cells derived from umbilical cord (UC-MSCs) using centrifugation method. The isolated mitochondria were stained with Mitotracker CMX Ros, and the concentration and total amount of the isolated mitochondria was confirmed by BCA quantification method, and 0 ug, 1 ug, 5 ug, 10 ug, 50 ug, 100 ug of mitochondria were injected into SNU-484 cells, a gastric cancer cell line, using centrifugation method. As a result of the experiment, it was confirmed that the degree of mitochondria injected into the cells was concentration-dependent on the amount of mitochondria by a fluorescence microscope (FIG. 57).

### Example 12. Confirmation of Influence of Normal Mitochondria on Cancer Cells

The following experiment was conducted to investigate how mitochondria derived from normal cells have an influence on the proliferation of cancer cells and ROS production. First, liver cells (WRL-68), fibroblasts, and mesenchymal stem cells derived from umbilical cord (UC-MSCs) were selected as mitochondria donor cells. The mitochondria were isolated from the cells by a centrifugation fractionation method, respectively. The cancer cell used as a mitochondria recipient cell was a skin epidermal cancer cell, A431 cell line. In this case, the mitochondria were delivered into the skin epidermal cancer cells using centrifugal force according to the concentration (see Korean Patent Appln. No. 10-2017-0151526).

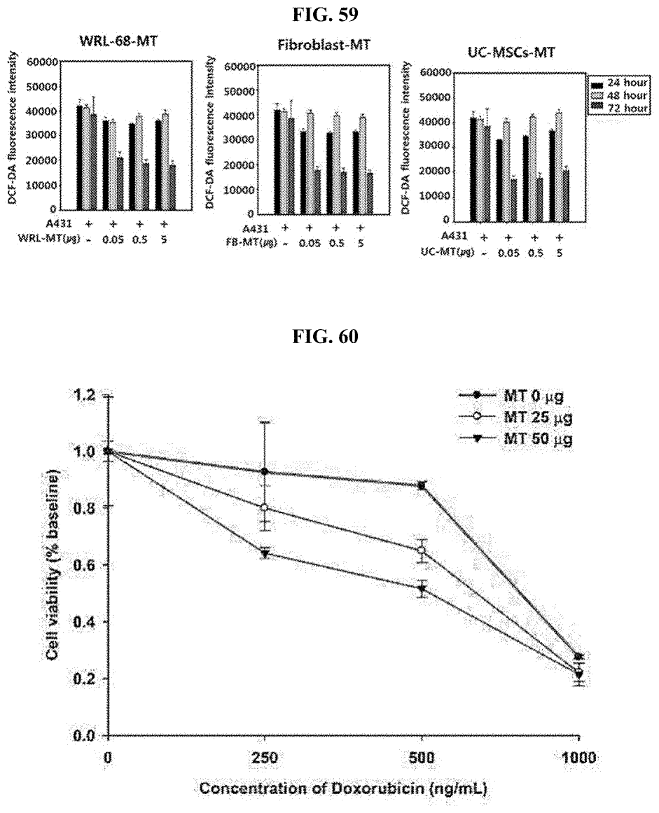

After 24, 48, and 72 hours after introduction, the proliferation of skin epidermal cancer cells and the production of reactive oxygen species (ROS) were observed. As a result, it was confirmed that when mitochondria obtained from normal cells from various origins were injected into cancer cells, there was an effect of inhibiting the proliferation of cancer cells depending on the concentration. In addition, it was confirmed that ROS production in cancer cells was inhibited depending on the concentration of normal mitochondria (FIGS. 58 and 59).

### Example 13. Confirmation of Influence of Normal Mitochondria on Drug Resistance

It was investigated how to influence on drug resistance, the expression of an antioxidant gene, cancer metastasis (metastasis), which are features of cancer cells, when mitochondria derived from normal cells were injected into cancer cells, by the following methods. First, normal liver cells (WRL-68) were set as mitochondria donor cells, and the mitochondria were isolated from the cells by centrifugation fractionation method, and the mitochondria were used. HepG2 cells, a liver cancer cell line, were used as cancer cells used as mitochondria recipient cells. The mitochondria were delivered into the liver cancer cells using centrifugal force according to the concentration, and then it was confirmed that as a result of observation of the drug resistance to doxorubicin, an anticancer agent, cancer cell lines that received mitochondria showed higher drug sensitivity (FIG. 60).

### Example 14. Confirmation of Influence of Normal Mitochondria on Antioxidant Effect

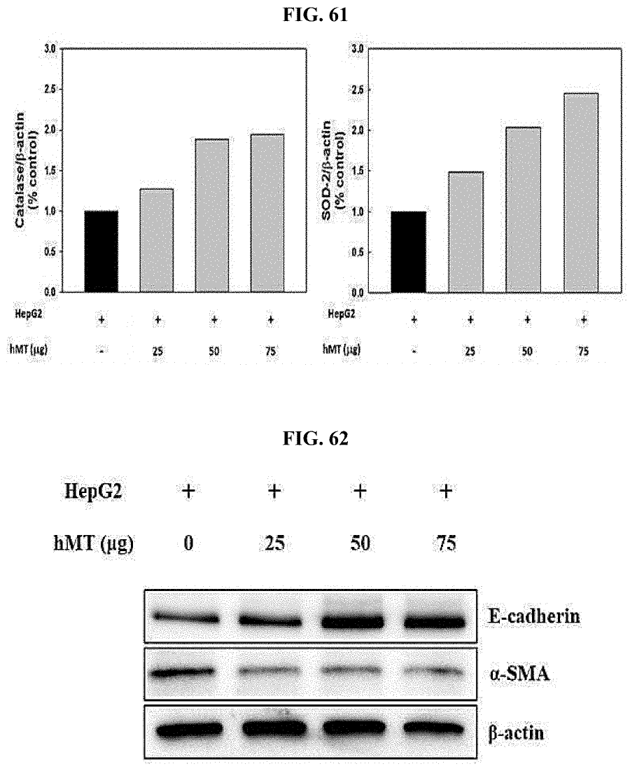

As the mitochondria isolated from normal cells were injected into HepG2 cells, a liver cancer cell line, according to the concentration, it was confirmed that the expression of enzyme catalase, an antioxidant protein, and SOD-2 (superoxide dismutase-2) genes in cancer cells were increased (FIG. 61).

### Example 15. Confirmation of Influence of Normal Mitochondria on Cancer Cell Metastasis

In relation to metastasis, it was confirmed whether there was the expression of α-smooth muscle actin (α-SMA) gene, one of the genes involved in EMT (epithelial to mesenchymal transition). In this case, it was found that, in the case of liver cancer cells that received mitochondria, the expression of α-SMA protein was significantly reduced depending on the concentration of mitochondria, compared to liver cancer cells that did not receive mitochondria. On the contrary, it was found that the E-cadherin protein, one of the cell adhesion proteins, was increased depending on the concentration of mitochondria (FIG. 62). It was confirmed that the changes of proteins known to be involved in the cancer metastasis are made by normal mitochondria injected into the cancer cells, and thus also influence the metastasis of cancer cells.

### Example 16. Confirmation of Loading of Recombinant Protein p53 on Foreign Mitochondrial Outer Membrane and Injection into Cells

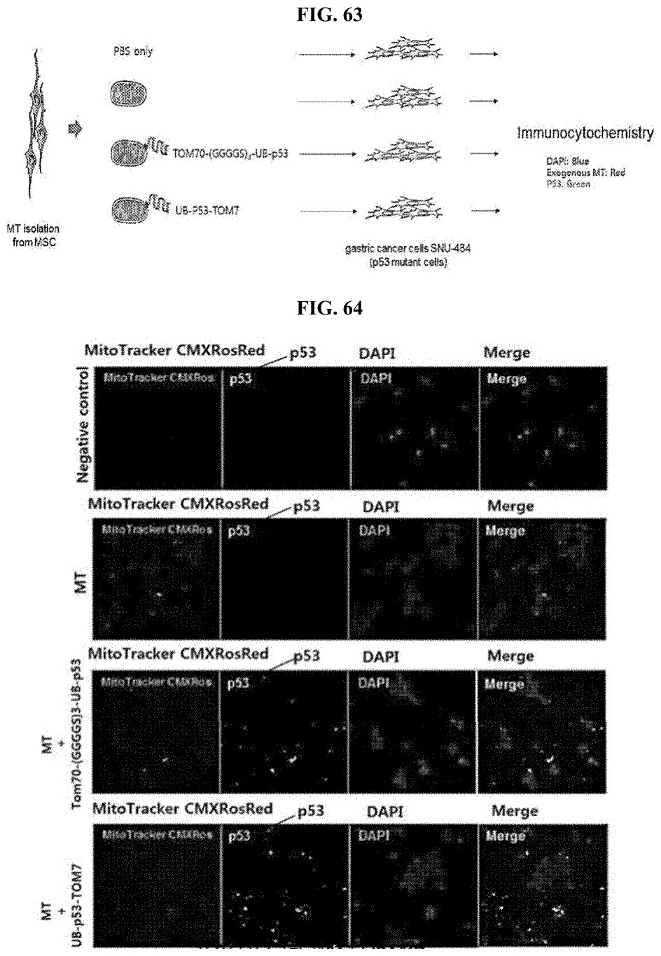

The mitochondria were isolated from mesenchymal stem cells derived from umbilical cord using centrifugation method, and then were stained with Mitotracker CMX Ros, and were mixed with the purified recombinant protein TOM70-(GGGGS)3-UB-p53 or UB-p53-TOM7, and were incubated at a ratio of 1:1 under a reaction condition of at 4° C. for 1 hour, and then were centrifuged to remove the unreacted proteins, and then were washed twice with buffer solution PBS, and then the mitochondria in the form to which p53 protein was bound were injected into SNU-484 cells, a gastric cancer cell line, by centrifugation method (FIG. 63). In this case, a control group was set to a group that did not use mitochondria and a group that used mitochondria alone. After one day of culture, the p53 protein loaded on the foreign mitochondria injected into the cells was observed with a fluorescence microscope using immunocytochemistry (ICC).

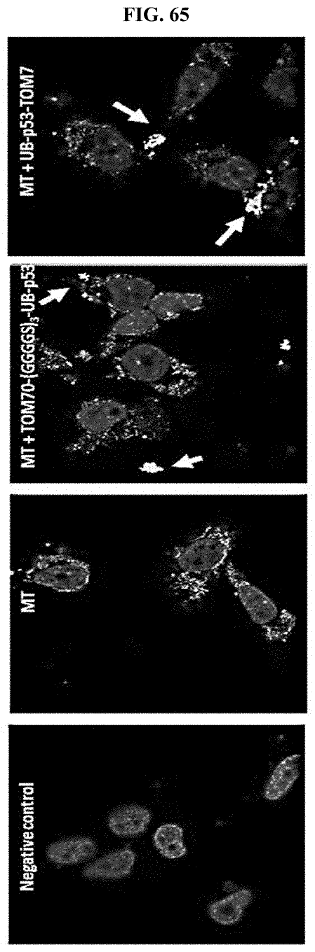

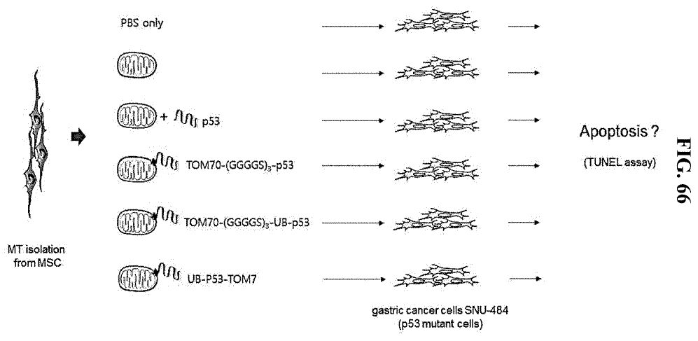

Rabbit anti-p53 antibody was used as a primary antibody, and Goat anti-rabbit IgG Alexa Fluor 488 was used as a secondary antibody. As a result, it was confirmed that TOM70-(GGGGS)3-UB-p53 (green stained) or UB-p53-TOM7 (green stained) protein loaded on the foreign mitochondria (red stained) was located in the cytoplasm in the cells that were injected along with the foreign mitochondria during injection into the cells (FIG. 64, 200 magnification and FIG. 65, 400 magnification). As a result, it was found that the recombinant protein was easily injected into the cell via the mitochondria.

### Example 17. Confirmation of Activity of p53 Loaded Mitochondria in Cancer Cell Line

### Example 17.1. Confirmation of Apoptosis Ability of p53 Loaded Foreign Mitochondria Injected into Cells Using Gastric Cancer Cell Line

The mitochondria isolated from mesenchymal stem cells derived from umbilical cord using centrifugation method were mixed with the recombinant protein TOM70-(GGGGS)3-UB-p53 or UB-p53-TOM7 that was purified from E. coli, and were allowed to be bound at a ratio of 1:1 under a reaction condition of at 4° C. for 1 hour. As a control group, the UB-p53 protein that does not comprise TOM70 and TOM70-(GGGGS)3-p53 that does not comprise ubiquitin were used. The proteins unbound were removed by centrifugation and PBS washing process, and the mitochondria to which proteins were bound were injected into a gastric cancer cell line SNU-484, which lacks p53 ability due to the variation of p53 gene, by centrifugation (FIG. 66). After one day of culture, the fixation was performed with 4% paraformaldehyde for 1 hour, and then the permeabilization of cells was induced using permeabilization solution (0.1% sodium citrate buffer comprising 0.1% Triton-X-100, pH 7.4), and reacted with TUNEL solution (In situ cell death detection kit, TMR RED, Roche) at 37° C. for 1 hour.

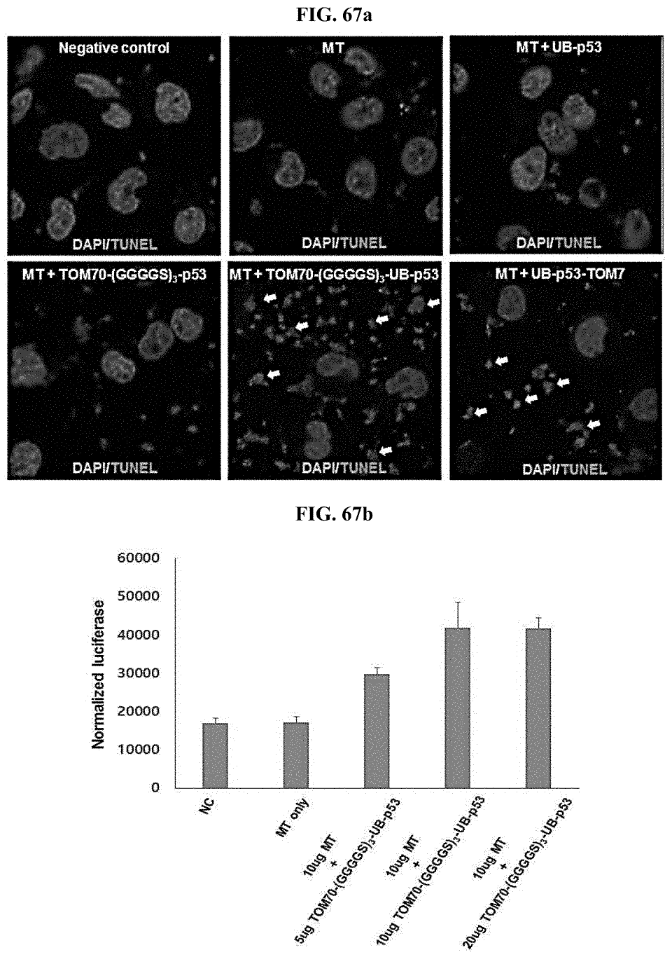

In the TUNEL analysis method, the portion where the fragmentation of nucleic acid (DNA fragmentation) occurred is stained in red color, indicating that apoptosis occurs. Compared to the control group, in the cells injected with the mitochondria to which TOM70-(GGGGS)3-ub-p53 or p53-TOM7 was bound, a large amount of red stained portion was found, unlike the control group, indicating that apoptosis occurred by the mitochondria to which TOM70-(GGGGS)3-UB-p53 or UB-p53-TOM7 was bound. In particular, it was confirmed that more apoptosis occurred in the mitochondria to which the protein in the form of TOM70-(GGGGS)3-UB-p53 was bound (FIG. 67a).

### Example 17.2. Confirmation of Apoptosis Ability of p53 Loaded Foreign Mitochondria to which Luciferase was Bound

In order to confirm whether the biological activity of the delivered TOM70-(GGGGS)3-UB-p53 protein in a recipient cell was maintained after the TOM70-(GGGGS)3-UB-p53 protein in the form bound to the mitochondria obtained in Example 5.2. above was delivered into the recipient cells, a cell-based analysis using a reporter gene was performed. Since the p53 protein is a transcription factor, a gene in which the base sequence RRRCWWGYYY (wherein R represents G or A, W represents A or T, and Y represents C or T) to which the p53 transcription factor can bind is repeated 6 times was synthesized with the following sequence. The base sequence of P53-promter-S is as follows (5′-GGG CAT GCT CGG GCA TGC CCG GGC ATG CTC GGG CAT GCC CGG GCA TGC TCG GGC ATG CCC-3′)(SEQ ID NO: 91), and the base sequence of P53-promter-AS is as follows (5′-GGG CAT GCC CGA GCA TGC CCG GGC ATG CCC GAG CAT GCC CGG GCA TGC CCG AGC ATG CCC-3′)(SEQ ID NO: 92).

5 ug of the synthesized gene P53-promter-S and 5 ug of the synthesized gene P53-promter-AS were incubated at 70° C. for 20 minutes to promote the synthesis of double helix gene, and then the phosphorylation reaction was induced using a polynucleotide T4 kinase enzyme. The double helix gene in which the phosphorylation was induced was inserted into a pGL3 vector cleaved by the restriction enzyme Sma I, and a gene in which the base sequence (RRRCWWGYYY) to which the p53 transcription factor can bind is repeated 6 times was allowed to be bound to luciferase, a reporter gene, to prepare the plasmid p6xp53-Luc. The plasmid p6xp53-Luc and the plasmid pRSVb-gal, a beta-galactosidase expression vector, were transformed into HEK293 cells, human renal cells, by lipofectamine method.

Subsequently, after 6 hours, the HEK293 cells were treated with a combination in which 10 ug of the mitochondria and 5 ug, 10 ug, and 20 ug of the TOM70-(GGGGS)3-UB-p53 protein was bound, respectively. In this case, as a control group, the cells were treated with 10 ug of the mitochondria to which PBS or the p53 protein was bound, respectively. The treated cells were cultured for 18 hours, and then the luciferase activity was measured and analyzed. In this case, in order to correct the efficiency of transformation, the luciferase value divided by the value obtained by measuring the activity of beta-galactosidase was determined as a corrected luciferase value.

It was confirmed that the luciferase value was increased in the cells treated with a combination in which 10 ug of the mitochondria and 5 ug, 10 ug, and 20 ug of the TOM70-(GGGGS)3-UB-p53 protein was bound, respectively. Thus, it was confirmed that the p53 protein entered into the cells and exhibited the activity (FIG. 67b).

### Example 18. Confirmation of Ability of RKIP Loaded Foreign Mitochondria Injected into Cells to Reduce Metastasis of Cancer Cell Line

The mitochondria isolated from mesenchymal stem cells derived from umbilical cord using centrifugation method were mixed with the purified recombinant protein TOM70-(GGGGS)3-UB-RKIP, and were allowed to be bound at a ratio of 1:1 under a reaction condition of at 4° C. for 1 hour. The mitochondria to which the protein was bound were injected by centrifugation into the breast cancer cell line MDA-MB-231, which is known to have increased metastasis ability due to a decrease in RKIP protein.

In order to confirm the ability of metastasis of cancer cells, a cell invasion assay using a transwell plate was performed. The transwell upper-chamber having a pore size of 8 μm was coated with matrigel for 30 minutes at 37° C. As a test group, MDA-MB-231 cells injected with mitochondria alone and MDA-MB-231 cells injected with mitochondria to which RKIP protein was bound were used. Each cell at 1×10⁵ cells was placed in a transwell upper chamber containing serum-free medium, and a medium comprising 10% bovine serum was placed in a lower-chamber. After culturing at 37° C. for 12 hours, the fixation was performed with 4% paraformaldehyde for 1 hour, and then the cells that passed through matrigel were stained with 1% crystal violet.

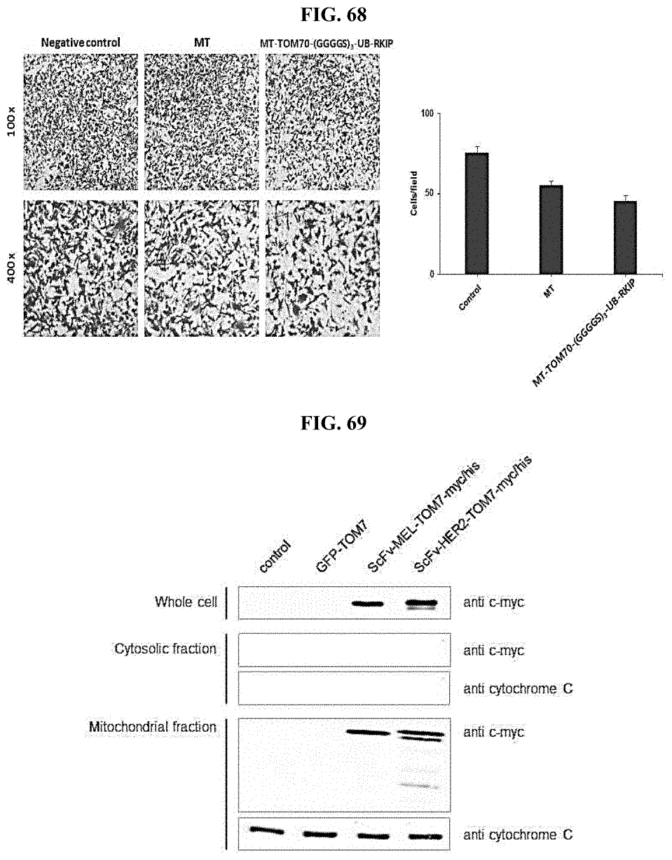

As a result of observation under a microscope, the cells stained in purple were observed in the membrane below the upper-chamber, and this can be said to be a process in which metastasis of cells occurred. It was confirmed that the cells stained in purple were reduced in the experimental group treated with mitochondria alone and the experimental group treated with mitochondria to which RKIP was bound, compared to the control group that was treated with nothing. Four parts were randomly selected, and then the number of stained cells was measured and was plotted on the graph (FIG. 68).

### IV. Confirmation of Delivery Rate of Modified Mitochondria to which Target Targeting Protein was Bound

### Example 19. Confirmation of Intracellular Expression of Single Chain Variable Fragment (ScFv) Antibody for Targeting Cancer Cells and Confirmation of Binding with Mitochondria in Cells

In order to express pCMV-ScFv-HER2-TOM7 or pCMV-ScFv-MEL-TOM7 or pCMV-ScFv-PD-L1-TOM7 in animal cells, the DNA was transfected into CHO cells using Lipofectamine LTX and PLUS or Lipofectamine 2000. GFP-TOM7 DNA was used as a control group. In order to confirm that they are expressed in a cell and binds to mitochondria in the same cell, cytosol and mitochondria were isolated from the transfected cells using centrifugation method and adjusted to the same protein amount using a BCA assay, and then PAGE electrophoresis was performed, and then the results were observed by Western blot. Monoclonal c-myc antibody was used as a primary antibody, and Anti-mouse IgG HRP was used as a secondary antibody.

The bands of ScFv-HER2-TOM7 or ScFv-MEL-TOM7 proteins were identified at the expected size of 35 kDa. Based on that all were identified in the mitochondrial layer, it could be expected that the transfected and expressed proteins were bound to mitochondria in cells by TOM7 (FIG. 69).

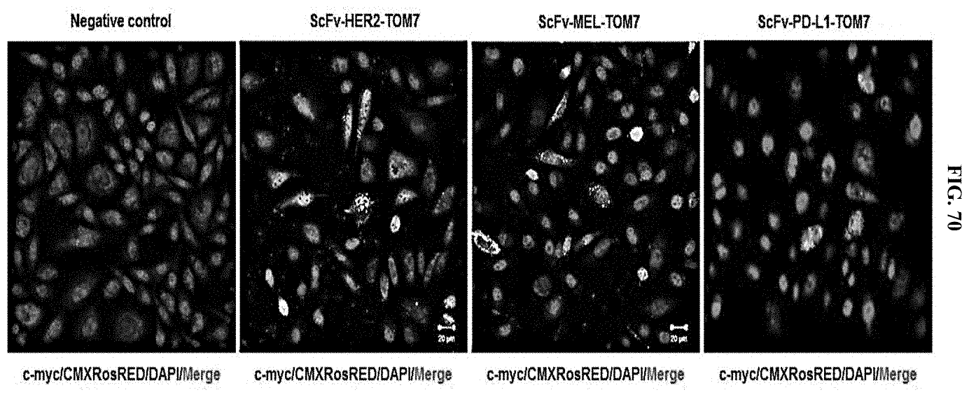

Next, in order to confirm the binding of the target protein expressed in a cell to the mitochondria in the same cell, the ScFv-HER2-TOM7, ScFv-MEL-TOM7 or ScFv-PD-L1-TOM7 protein expressed in the cell was observed with a fluorescence microscope using an immunocytochemistry (ICC) experimental method. Monoclonal c-myc antibody was used as a primary antibody, and Goat anti-mouse IgG Alexa Fluor 488 was used as a secondary antibody. The mitochondria in the cell were stained with Mitotracker CMX Ros. As a result, it was confirmed that the expressed ScFv-HER2-TOM7, ScFv-MEL-TOM7 or ScFv-PD-L1-TOM7 proteins were colocalized with the mitochondria and were bound to the mitochondria in the cell (FIGS. 70 and 71).

### Example 20. Isolation of Mitochondria to which Single Chain Variable Fragment Antibody for Targeting Cancer Cells was Bound and Comparison of Injection of Mitochondria in Gastric Cancer Cell Line

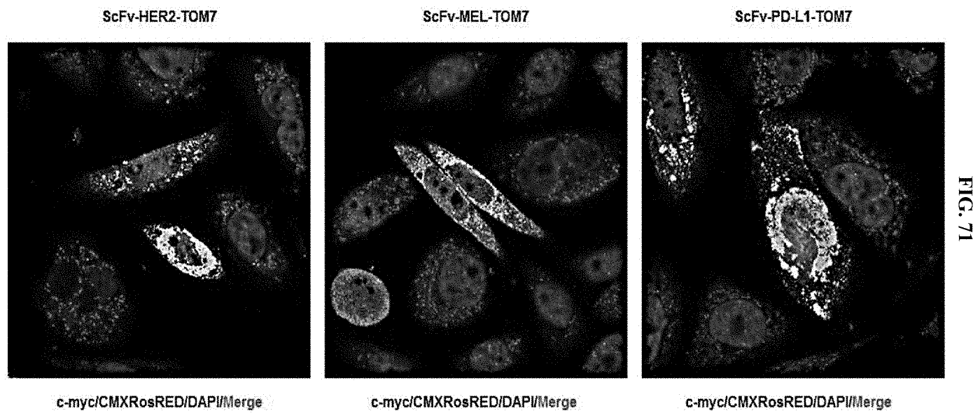

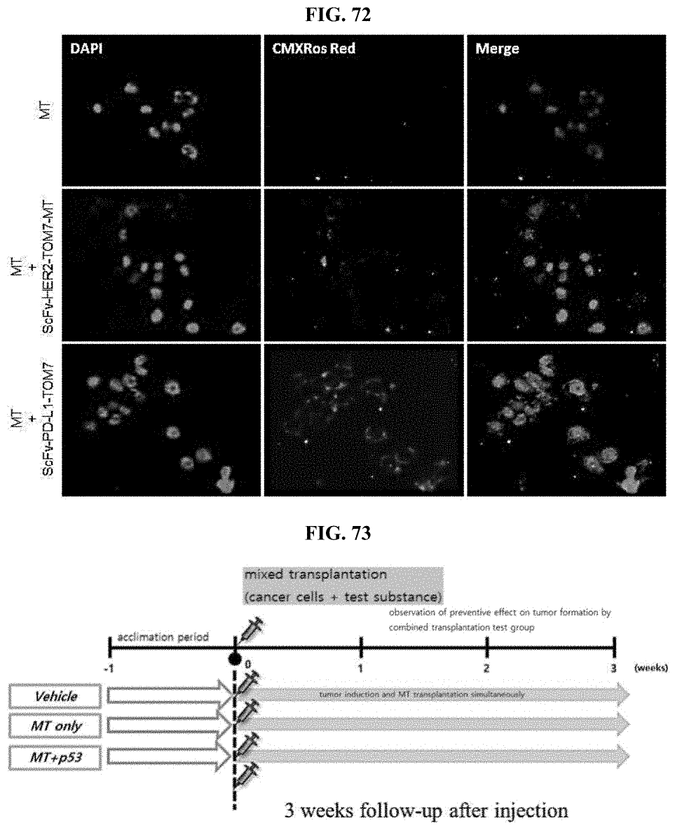

The mitochondria were isolated from CHO cells into which pCMV-ScFv-HER2-TOM7 or pCMV-ScFv-PD-L1-TOM7 was transfected. As a control group, the mitochondria of CHO cells which were not transformed were isolated and used. The mitochondria isolated from each cell were stained with Mitotracker CMX Ros. SNU-484, a gastric cancer cell line, was treated with the same amount of mitochondria, and the next day, the degree of mitochondria injected into the cells were compared and confirmed using a fluorescence microscope. It was confirmed that, compared to the control group, the mitochondria to which ScFv-HER2-TOM7 or ScFv-PD-L1-TOM7 was bound were injected into cancer cells more than the mitochondria obtained from the control group (FIG. 72). Therefore, it was found that the mitochondria to which the target protein was bound is more easily injected into cancer cells when using mitochondria alone.

### VI. Confirmation of In Vivo Activity of Modified Mitochondria to which Active Protein was Bound

### Example 21. Construction of Xenograft Model (SNU-484) and Administration of Test Substance

### Example 21.1. Preparation of Cancer Cells

On the day of the experiment, SNU-484 cell line, a gastric cancer cell line, was prepared to be 5×10⁶ cells per mouse. The medium of the cells was removed, and then PBS was added to wash the cells. The cells were dissociated using a Trypsin-EDTA solution, and then the cells were placed in a 50 mL tube, and washed twice with PBS buffer solution, and then 20 mL of PBS was added, and the number of cells and viability were measured. Based on the measured number of cells, the number of cells was adjusted to be 5×10⁶ cells per mouse, and the cells were prepared by dividing them into groups. The volume to be transplanted per mouse was adjusted to the same amount of 100 μL. As a control group, 100 μL of a cancer cell alone group was prepared.

### Example 21.2. Preparation of Test Substance

The mitochondria isolated from umbilical cord blood mesenchymal stem cells as described above were prepared for the transplantation at an amount of 50 μg per mouse based on the protein concentration. In the case of a group to which mitochondria alone were administered, the mitochondria were prepared by mixing well with 100 μL of PBS in which cancer cells were mixed. In the case of the modified mitochondria group, the TOM70-(GGGGS)3-UB-p53 protein was mixed together in a concentration ratio of 1:1 with the amount of mitochondria prepared in the Eppendorf tube before mixing with cancer cells, and was stood at ambient temperature for 1 hour. After the reaction time was over, the supernatant was removed after centrifugation at 20,000×g for 10 minutes, and the pellet of the mitochondria (MT+TOM70-(GGGGS)3-UB-p53) to which a protein was bound was obtained. It was washed twice using PBS buffer solution, and then the mitochondria (MT+TOM70-(GGGGS)3-UB-p53) to which p53 protein was bound were prepared by mixing well with 100 μL of PBS in which cancer cells were mixed.

### Example 21.3. Preparation of Experimental Animal and Transplantation of Test Substance

For the transplantation sample prepared by the groups, matrigel (BD) was added at the same amount as that of PBS and lightly mixed with the cells to prepare 200 μL of test substance per mouse. In this case, all operations were performed on ice. For the model construction, Balb/c nude mice (female, 7-week old) were purchased from RAONBIO, and anesthetized by the inhalation of isoflurane for the transplantation of cancer cells, and then the right back area (on the basis of animal) was sterilized with an alcohol swab. Thereafter, 200 μL was administered subcutaneously to the right back area of the experimental animal using a 1 mL syringe containing the injection solution. After administration, the weight of the animal and the size of the tumor were measured twice a week, and the analysis of the results proceeded while observing up to 3 weeks (FIG. 73).

### Example 21.4. Confirmation of Tumor Formation

The volume of the tumor was calculated by measuring the long axis length and short axis length of the tumor and applying them to the following equation.

long axis X short axis X short axis X 0.5=tumor volume (mm\hat{ }3) <Mathematical equation 1>

### Example 21.5. Observation of Physiological and Morphological Change

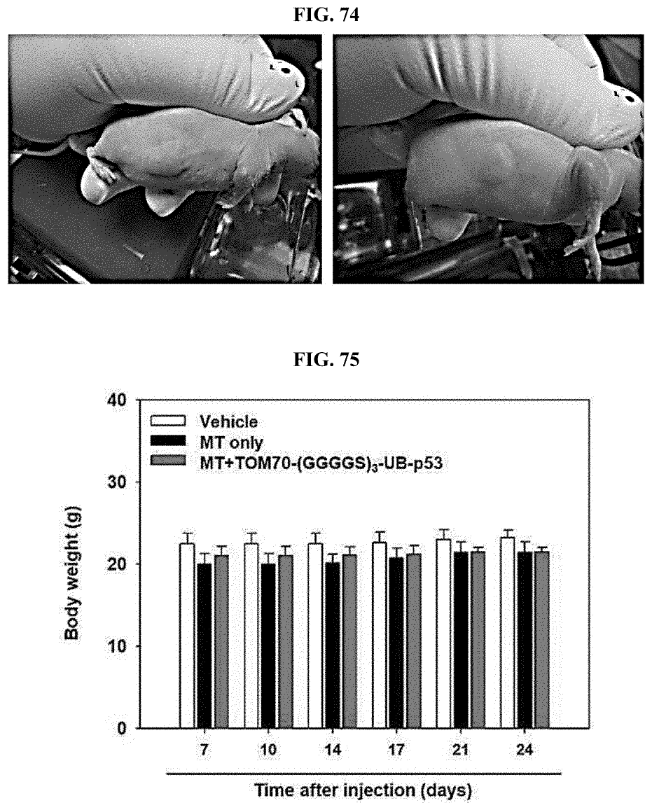

In order to observe the physiological and morphological change of mice by administration of anticancer candidates, the changes in body weight and the tumor size were measured twice a week from the time of administration of cancer cells and test substances (FIG. 74).

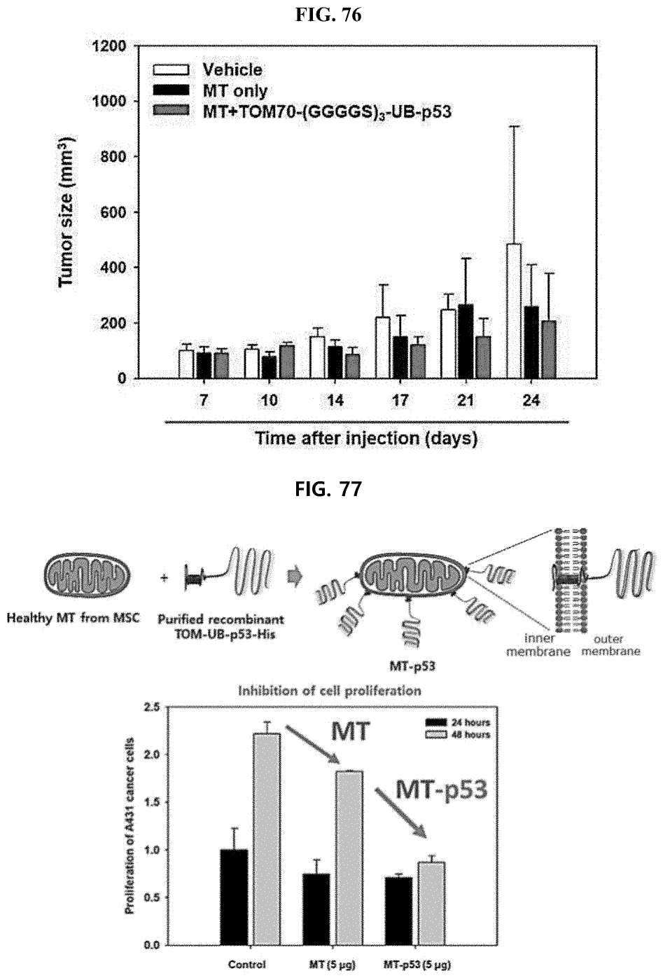

The weight of the mouse was measured using a scale, and the change by group was analyzed using the values measured twice a week (FIG. 75). It was confirmed that there was no significant difference in the change in body weight for 3 weeks between the group into which mitochondria were not injected, the group to which mitochondria were administered alone, and the group into which modified mitochondria were injected. The size of the tumor was calculated by measuring the length of the long axis (length) and short axis (width) of the tumor using a caliper, and then applying them to the equation of Mathematical equation 1 above. The change by group was analyzed using the values measured twice a week (FIG. 76). It was found that the size of the tumor was significantly increased over time in the group that was not treated with mitochondria, whereas in the case of mice that were administered with mitochondria, the increase in the size of the tumor slowed down over time. In addition, it was confirmed that the increase in the size of the tumor was significantly lowered in the group that was administered with mitochondria on which p53 protein was loaded, compared to the group that was administered with mitochondria alone (FIG. 76).

### Example 22. Confirmation of Effect of Modified Mitochondria on Inhibiting Proliferation of Skin Cancer Cells

The mitochondria obtained above to which p53 was bound were delivered to A431 cells, which are skin cancer cells, by centrifugation method, and then the proliferation of A431 cells was observed. In this case, physiological saline was used as a control group, and an equivalent amount of mitochondria to which p53 protein was not fused was used as a control test group. It was confirmed that the mitochondria on which p53 protein, a protein inducing apoptosis, was loaded can significantly inhibit the proliferation of A431 cells, compared to the control group and the group in which only mitochondria were used (FIG. 76).

### V. Confirmation of Activity of Isolated Mitochondria

### Example 23. Confirmation of Function of Isolated Mitochondria: ATP Content

In order to isolate the intracellular mitochondria from mesenchymal stem cells derived from umbilical cord (UC-MSCs), homogenization was performed using a syringe to break the cells, and then continuous centrifugation was performed to obtain the mitochondria. In order to confirm the function of the isolated mitochondria, the mitochondria protein concentration of the isolated mitochondria was quantified through a BCA assay to prepare 5 μg of the mitochondria. The amount of ATP in the mitochondria was confirmed using a CellTiter-Glo luminescence kit (Promega, Madison, Wis.).

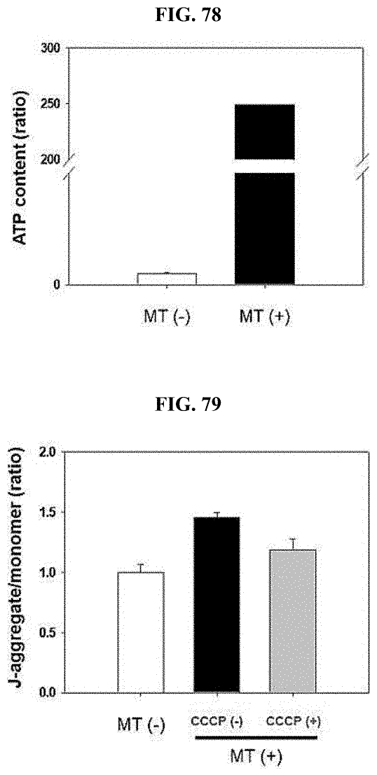

The prepared mitochondria were mixed in 100 ul of PBS, and then prepared in a 96 well plate, and compared to 100 ul of PBS that did not contain mitochondria as a control group. 100 μL of the test solution that was included in the kit was added in the same manner, and reacted and mixed well for 2 minutes in a stirrer, and then reacted at ambient temperature for 10 minutes, and then the amount of ATP was measured using a Luminescence microplate reader. It was confirmed that ATP was increased when mitochondria was included compared to the control group, and the function of mitochondria was confirmed (FIG. 78).

### Example 24. Confirmation of Function of Isolated Mitochondria: Membrane Potential

In order to confirm the membrane potential of the isolated mitochondria, JC-1 dye (molecular probes, cat no. 1743159) dye was used. The prepared mitochondria were mixed in 50 μL of PBS, and then prepared in a 96 well plate. PBS (50 μL) group that did not contain mitochondria as a control group and CCCP (R&D systems, CAS 555-60-2) treatment group were prepared. CCCP, an ionophore of mitochondria, inhibits mitochondrial function by depolarization of the mitochondrial membrane potential. The CCCP group was reacted with the isolated mitochondria at 50 μM for 10 minutes at room temperature.

Thereafter, it was reacted with JC-1 dye (2 μM) in the same manner, and then the absorbance was measured using a property having a different spectrum according to the concentration generated by a change in the membrane potential. At low concentrations, it exists as a monomer and exhibits green fluorescence, and at high concentrations, dye aggregates (J-aggregate) to exhibit red fluorescence. The mitochondrial membrane potential was analyzed by calculating the ratio of green absorbance to red absorbance. After the reaction was completed, the mitochondria membrane potential was measured using a fluorescence microplate reader (Monomer: Ex 485/Em 530, J-aggregate: Ex 535/Em 590). The results are shown in FIG. 79.

### Example 25. Confirmation of Degree of Damage of Isolated Mitochondria Through Confirmation of mROS Production

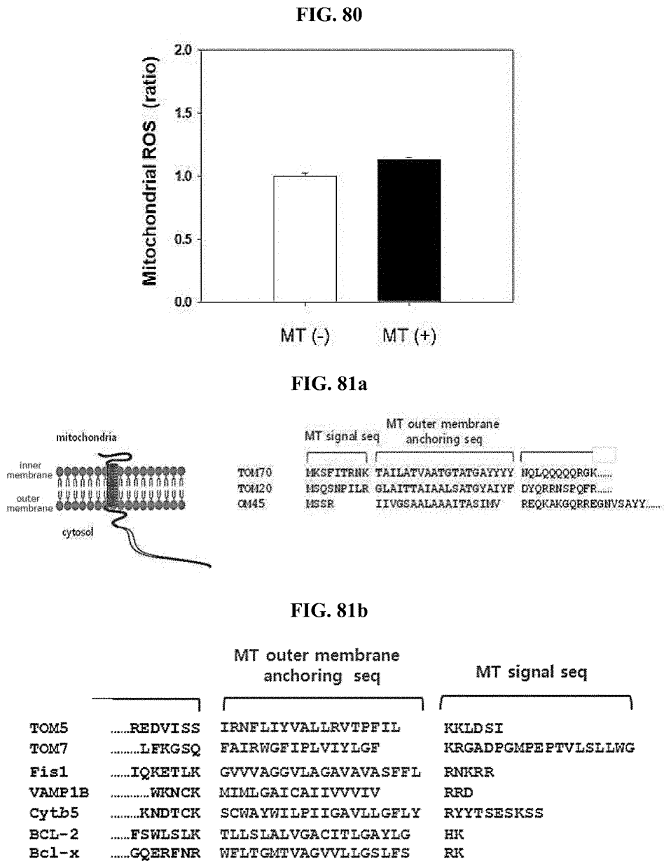

In order to confirm whether 5 μg of mitochondria prepared as described above is damaged, a MitoSOX red indicator (Invitrogen, cat no. M36008) dye capable of analyzing mitochondrial reactive oxygen species in the isolated mitochondria was used. The prepared mitochondria were mixed in 50 μL of PBS, and then prepared in a 96 well plate, and compared to 50 μL of PBS that did not contain mitochondria as a control group. The MitoSOX red dye was mixed in 50 μL of PBS to be a concentration of 10 μM, and placed in a 96-well plate (final concentration 5 μM), and then reacted in a 37° C., CO₂ incubator for 20 minutes. After the reaction was completed, the amount of ROS in mitochondria was measured using a microplate reader (Ex 510/Em 580). The results are shown in FIG. 80.

### VI. Confirmation of Dissociation of Desired Protein Bound to Mitochondrial Outer Membrane Protein Outside and Inside Cells

### Example 26. Confirmation of Dissociation of Desired Protein Bound to Mitochondrial Outer Membrane Protein Outside Cells

In order to obtain a desired protein in a free form when the active protein bound with the mitochondria was injected into the cell, a fusion protein (TOM70-UB-p53 or TOM-UB-GFP) in the form in which ubiquitin protein was inserted between the mitochondrial outer membrane protein and the desired protein was prepared from E. coli. In order to confirm whether ubiquitin sequence was cleaved by UBP1, a ubiquitin cleaving enzyme, the recombinant fusion protein TOM70-UB-p53 was reacted with the UBP1 enzyme at 37° C. for 1 hour.

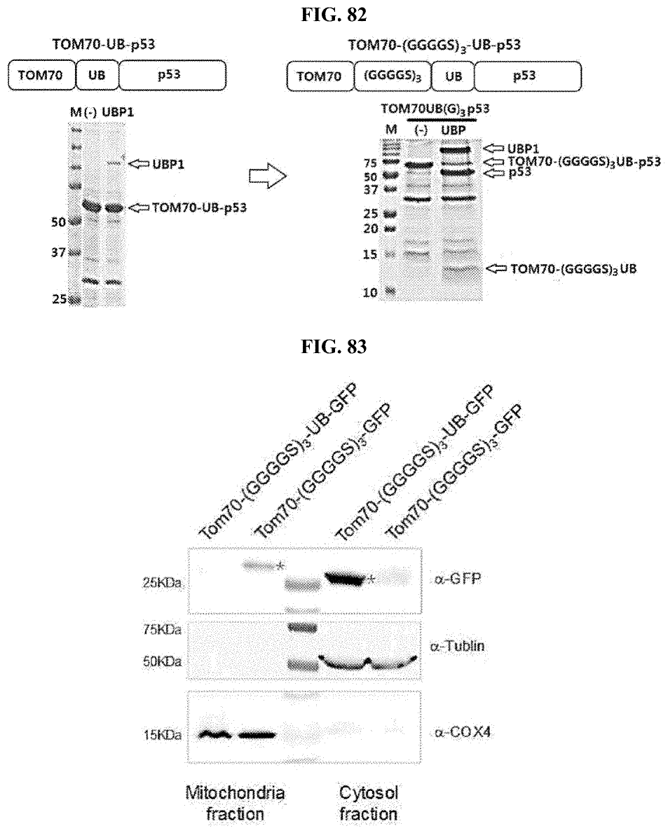

Thereafter, as a result of the analysis by SDS-PAGE electrophoresis, it was confirmed that the dissociation of the ubiquitin protein from the fusion protein did not occur at all by UBP1. This was considered to be an interference phenomenon of the mitochondrial outer membrane protein structurally, and thus, a linker protein composed of the amino acid glycine and serine was inserted between the mitochondrial outer membrane protein and the ubiquitin protein, and a new fusion protein (TOM70-(GGGGS)3-UB-p53 or TOM70-(GGGGS)3-UB-GFP) was obtained by purification from E. coli, and then reacted with UBP1 enzyme at 37° C. for 1 hour as described above. As a result, it was confirmed through SDS-PAGE electrophoresis that the 3′ end of ubiquitin was cleaved by UBP1 enzyme, and only p53 protein was dissociation as expected (FIG. 82).

### Example 26. Confirmation of Dissociation of Desired Protein Bound to Mitochondrial Outer Membrane Protein Inside Cells

When the fusion protein (TOM70-(GGGGS)3-UB-p53 or TOM70-(GGGGS)3-UB-GFP) obtained in the above example enters the cell in a state of being bound to mitochondria, it was observed whether the active protein was dissociated by the ubiquitin cleaving enzyme present in the cell. First, the mitochondria obtained from umbilical cord blood mesenchymal cells and the fusion protein TOM70-(GGGGS)3-UB-GFP were reacted for 1 hour in a microtube to allow to be bound, and then the unbound fusion protein was removed by centrifugation and then washed twice with a PBS buffer solution. In this case, the fusion protein (TOM70-(GGGGS)3-GFP) from which ubiquitin was removed was used as a control group.

Thereafter, the protein bound to the mitochondria was injected into MDA-MB-231 cells, a breast cancer cell line, by centrifugation method. After one day, MDA-MB-231 cells were crushed and fractionated into a mitochondrial part and a cytosolic part, respectively, using differentiated gravity. As a result of analysis by SDS-PAGE electrophoresis and Western blot analysis, it was found that in the case of the fusion protein in which ubiquitin was included, GFP proteins dissociated from mitochondrial outer membrane protein, a linker protein and ubiquitin were mostly detected in a cytosolic part, and it was found that in the case of the fusion protein from which ubiquitin was removed, GFP proteins in the form to which mitochondrial outer membrane protein and a linker protein were bound were mostly detected in a mitochondrial fractional part (FIG. 83).

As a result, it was found that when the mitochondrial outer membrane protein-linker-ubiquitin-active protein bound to the mitochondria was injected into the cells, the ubiquitin and active protein connection site was cleaved, and the dissociated active protein was released to the cytoplasm, and it was found that through this, mitochondria can be used as a delivery vehicle as one of methods for effectively delivering a useful protein into cells.

1. A modified mitochondria in which a foreign protein is bound to the outer membrane of the mitochondria, wherein the foreign protein is a fusion protein comprising a mitochondria anchoring peptide and a desired protein capable of functioning inside and outside the cell.

2. The modified mitochondria according to claim 1, wherein the mitochondria are isolated from eukaryotic cells, tissues, or platelets.

3.-5. (canceled)

6. The modified mitochondria according to claim 1, wherein the foreign protein is bound to the outer membrane of the mitochondria by a mitochondria anchoring peptide.

7. The modified mitochondria according to claim 6, wherein the mitochondria anchoring peptide comprises an N terminal region or a C terminal region of a protein present in the mitochondrial membrane protein.

8. The modified mitochondria according to claim 7, characterized in that the N terminal region or the C terminal region of the protein present in the mitochondrial membrane protein is located on the outer membrane of the mitochondria.

9. The modified mitochondria according to claim 7, characterized in that the protein present in the mitochondrial membrane protein is any one selected from the group consisting of TOM20, TOM70, OM45, TOM5, TOM6, TOM7, TOM22, Fis1, Bcl-2, Bcl-x and VAMP1B.

10. The modified mitochondria according to claim 7, characterized in that the anchoring peptide comprises an N terminal region of any one selected from the group consisting of TOM20, TOM70 and OM45.

11. The modified mitochondria according to claim 7, characterized in that the mitochondria anchoring peptide comprises a C terminal region of any one selected from the group consisting of TOM5, TOM6, TOM7, TOM22, Fis1, Bcl-2, Bcl-x and VAMP1B.

12. (canceled)

13. The modified mitochondria according to claim 1, wherein the desired protein is any one selected from the group consisting of an active protein exhibiting an activity in a cell, a protein present in a cell, and a protein having the ability to bind to a ligand or receptor present in a cell membrane.

14. The modified mitochondria according to claim 13, wherein the desired protein is any one selected from the group consisting of p53, Granzyme B, Bax, Bak, PDCD5, E2F, AP-1(Jun/Fos), EGR-1, Retinoblastoma(RB), phosphatase and tensin homolog(PTEN), E-cadherin, Neurofibromin-2(NF-2), poly[ADP-ribose] synthase 1(PARP-1), BRCA-1, BRCA-2, Adenomatous polyposis coli(APC), Tumor necrosis factor receptor-associated factor(TRAF), RAF kinase inhibitory protein(RKIP), p16, KLF-10, LKB1, LHX6, C-RASSF, DKK-3PD1, Oct3/4, Sox2, Klf4, and c-Myc.

15. The modified mitochondria according to claim 1, wherein the foreign protein is a desired protein bound to the N terminal region of TOM20, TOM70 or OM45.

16. The modified mitochondria according to claim 15, wherein the foreign protein is bound in the following order:
N terminal-N terminal region of TOM20, TOM70 or OM45-desired protein-C terminal.

17. The modified mitochondria according to claim 16, wherein the foreign protein further comprises an amino acid sequence recognized by a proteolytic enzyme in eukaryotic cells, or ubiquitin or a fragment thereof between the anchoring peptide and the desired protein.

18. The modified mitochondria according to claim 17, wherein the ubiquitin fragment comprises the C terminal Gly-Gly of an amino acid sequence of SEQ ID NO: 71, and comprises 3 to 75 amino acids consecutive from the C terminal.

19. The modified mitochondria according to claim 17, wherein the foreign protein further comprises a linker between a desired protein and ubiquitin or a fragment thereof.

20.-22. (canceled)

23. The modified mitochondria according to claim 13, wherein the protein having the ability to bind to a ligand or receptor present in a cell membrane is a ligand or receptor present on the surface of a tumor cell.

24. The modified mitochondria according to claim 23, wherein the ligand or receptor present on the surface of a tumor cell is any one selected from the group consisting of CD19, CD20, melanoma antigen E(MAGE), NY-ESO-1, carcinoembryonic antigen(CEA), mucin 1 cell surface associated(MUC-1), prostatic acid phosphatase(PAP), prostate specific antigen(PSA), survivin, tyrosine related protein 1(tyrp1), tyrosine related protein 1(tyrp2), Brachyury, Mesothelin, Epidermal growth factor receptor(EGFR), human epidermal growth factor receptor 2(HER-2), ERBB2, Wilms tumor protein(WT1), FAP, EpCAM, PD-L1, ACPP, CPT1A, IFNG, CD274, FOLR1, EPCAM, ICAM2, NCAM1, LRRC4, UNC5H2 LILRB2, CEACAM, Nectin-3, or a combination thereof.

25. The modified mitochondria according to claim 1, wherein the foreign protein is bound to a C terminal region of any one selected from the group consisting of TOM5, TOM6, TOM7, TOM22, Fis1, Bcl-2, Bcl-x and VAMP1B.

26. The modified mitochondria according to claim 25, wherein the foreign protein is bound in the following order:
N terminal-desired protein-C terminal region of any one selected from the group consisting of TOM5, TOM6, TOM7, TOM22, Fis1, Bcl-2, Bcl-x and VAMP1B-C terminal.

27. The modified mitochondria according to claim 26, wherein the foreign protein further comprises a linker between the desired protein and the C terminal region of any one selected from the group consisting of TOM5, TOME, TOM7, TOM22, Fis1, Bcl-2, Bcl-x and VAMP1B.

28.-30. (canceled)

31. A pharmaceutical composition comprising a modified mitochondria according claim 1 as an active ingredient.

32. The pharmaceutical composition according to claim 31, wherein the pharmaceutical composition is for the prevention or treatment of cancer.

33. The pharmaceutical composition according to claim 32, wherein the cancer is any one selected from the group consisting of gastric cancer, liver cancer, lung cancer, colorectal cancer, breast cancer, prostate cancer, ovarian cancer, pancreatic cancer, cervical cancer, thyroid cancer, larynx cancer, acute myeloid leukemia, brain tumor, neuroblastoma, retinoblastoma, head and neck cancer, salivary gland cancer and lymphoma.

34. A method of delivering a protein to a cell, comprising administering a modified mitochondria comprising a foreign protein capable of functioning inside and outside the cell, thereby resulting in intracellular and extracellular delivery of the foreign protein.

35. The method according to claim 34, wherein the foreign protein comprises a mitochondrial outer membrane anchoring peptide, and is bound to the outer membrane of the mitochondria by the outer membrane anchoring peptide, and is delivered inside and outside the cell.

36. A fusion protein comprising a mitochondrial outer membrane anchoring peptide and a desired protein capable of functioning inside and outside the cell, wherein the mitochondrial outer membrane anchoring peptide is any one selected from the group consisting of TOM20, TOM70, OM45, TOM5, TOM6, TOM7, TOM22, Fis1, Bcl-2, Bcl-x and VAMP1B.

37. The fusion protein according to claim 36, wherein the mitochondrial outer membrane anchoring peptide comprises an N terminal or C terminal sequence of a protein present in the outer membrane of mitochondria.

38. (canceled)

39. The fusion protein according to claim 36, wherein the desired protein is any one selected from the group consisting of p53, Granzyme B, Bax, Bak, PDCD5, E2F, AP-1(Jun/Fos), EGR-1, Retinoblastoma(RB), phosphatase and tensin homolog(PTEN), E-cadherin, Neurofibromin-2(NF-2), poly[ADP-ribose] synthase 1(PARP-1), BRCA-1, BRCA-2, Adenomatous polyposis coli(APC), Tumor necrosis factor receptor-associated factor(TRAF), RAF kinase inhibitory protein(RKIP), p16, KLF-10, LKB1, LHX6, C-RASSF, DKK-3PD1, Oct3/4, Sox2, Klf4, and c-Myc.

40. The fusion protein according to claim 36, wherein when the mitochondrial outer membrane anchoring peptide is TOM20, TOM70 or OM45, the mitochondrial outer membrane anchoring peptide and the desired protein are bound from the N terminal to the C terminal.

41. The fusion protein according to claim 36, wherein when the mitochondrial outer membrane anchoring peptide is any one selected from the group consisting of TOM5, TOM6, TOM7, TOM22, Fis1, Bcl-2, Bcl-x and VAMP1B, the desired protein and the mitochondrial outer membrane anchoring peptide are bound from the N terminal to the C terminal.

42. The fusion protein according to claim 36, wherein the fusion protein further comprises ubiquitin or a fragment thereof between the mitochondrial outer membrane anchoring peptide and the desired protein.

43. The fusion protein according to claim 36, wherein the fusion protein further comprises an amino acid sequence recognized by a proteolytic enzyme in eukaryotic cells between the mitochondrial outer membrane anchoring peptide and the desired protein.

44.-45. (canceled)

46. A fusion protein comprising a target targeting protein having the ability to bind to a ligand or receptor present in a cell membrane and a mitochondrial outer membrane anchoring peptide, wherein the mitochondrial outer membrane anchoring peptide is any one selected from the group consisting of TOM5, TOM6, TOM7, TOM22, Fis1, Bcl-2, Bcl-x and VAMP1B.

47. (canceled)

48. The fusion protein according to claim 46, wherein the target targeting protein having the ability to bind to a ligand or receptor present in a cell membrane and the mitochondrial outer membrane anchoring peptide are bound from the N terminal to the C terminal.

49. The fusion protein according to claim 48, wherein the target targeting protein having the ability to bind to a ligand or receptor present in a cell membrane is an antibody or a fragment thereof.

50. The fusion protein according to claim 49, wherein the fragment of the antibody is any one selected from the group consisting of Fab, Fab′, scFv and F (ab)2.

51.-53. (canceled)


SEQUENCE LISTING (95 sequences)

SEQ ID NO: 1 - DNA, 39 bp, Artificial Sequence
  T2p53 primer
aaaaaaccgcggtggtgaggagccgcagtcagatcctag

SEQ ID NO: 2 - DNA, 35 bp, Artificial Sequence
  Xp53 primer
aaaaaactcgagtgagtctgagtcaggcccttctg

SEQ ID NO: 3 - DNA, 1186 bp, Artificial Sequence
  nucleotides sequence coding p53
ccgcggtggtgaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 4 - DNA, 35 bp, Artificial Sequence
  NdeUB primer
ggattccatatgcaacttttcgtcaaaactctaac

SEQ ID NO: 5 - DNA, 27 bp, Artificial Sequence
  T2UB primer
atgaccaccgcggagtctcaacaccaa

SEQ ID NO: 6 - DNA, 1460 bp, Artificial Sequence
  nucleotides sequence coding UB-p53
atgggcagcagccatcatcatcatcatcacagcagcggcctggtgccgcgcggcagccatatgcaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtgaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgatag

SEQ ID NO: 7 - DNA, 59 bp, Artificial Sequence
  NdeTOM70 primer
gaattccatatgaaaagttttataactcggaataaaactgcaattttcgcaactgttgc

SEQ ID NO: 8 - DNA, 38 bp, Artificial Sequence
  TOM70-AS primer
ggtgcatactactattatcaaacttttcgtcaaaactc

SEQ ID NO: 9 - DNA, 36 bp, Artificial Sequence
  TOM70UB-S primer
ggctacgtatttatttccaacttttcgtcaaaactc

SEQ ID NO: 10 - DNA, 24 bp, Artificial Sequence
  T2UB-AS primer
ggcaccaccgcggagtctcaacac

SEQ ID NO: 11 - DNA, 1515 bp, Artificial Sequence
  nucleotides sequence coding TOM70-UB-p53
atgaaaagttttataactcggaataaaactgcaattttcgcaactgttgctgctacgggaaccgctattggtgcatactactattatcaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtgaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacctcgagcaccaccaccaccaccactag

SEQ ID NO: 12 - DNA, 59 bp, Artificial Sequence
  TOM70(G)3-AS primer
gcccccggatcctccacccccgcttccgccacctccataatagtagtatgcaccaatag

SEQ ID NO: 13 - DNA, 32 bp, Artificial Sequence
  (G)3UB-S primer
ggtggaggatccgggggcgcggaagccaaatc

SEQ ID NO: 14 - DNA, 32 bp, Artificial Sequence
  Xp53(noT) primer
aaaaaactcgaggtctgagtcaggcccttctg

SEQ ID NO: 15 - DNA, 1560 bp, Artificial Sequence
  nucleotides sequence coding TOM70-(GGGGS)3-UB- p53
atgaaaagttttataactcggaataaaactgcaattttcgcaactgttgctgctacgggaaccgctattggtgcatactactattatggaggtggcggaagcgggggtggaggatccgggggcggcggaagccaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtgaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacctcgagcaccaccaccaccaccactag

SEQ ID NO: 16 - DNA, 51 bp, Artificial Sequence
  B(G)3p53
ggtggaggatccgggggcggcggaagcgaggagccgcagtcagatcctagc

SEQ ID NO: 17 - DNA, 1335 bp, Artificial Sequence
  nucleotides sequence coding TOM70-(GGGGS)3-p53
atgaaaagttttataactcggaataaaactgcaattttcgcaactgttgctgctacgggaaccgctattggtgcatactactattatggaggtggcggaagcgggggtggaggatccgggggcggcggaagcgaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacctcgagcaccaccaccaccaccactag

SEQ ID NO: 18 - DNA, 32 bp, Artificial Sequence; identical to SEQ ID NO: 14
  Xp53(noT) primer

SEQ ID NO: 19 - DNA, 36 bp, Artificial Sequence
  XTOM7 primer
aaaaaactcgagtttgccattcgctggggctttatc

SEQ ID NO: 20 - DNA, 38 bp, Artificial Sequence
  LTOM7 primer
aaaaaagtcgacttatccccaaagtaggctcaaaacag

SEQ ID NO: 21 - DNA, 1578 bp, Artificial Sequence
  nucleotides sequence coding UB-p53-TOM7
atgggcagcagccatcatcatcatcatcacagcagcggcctggtgccgcgcggcagccatatgcaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtgaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgtccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacctcgagtttgccattcgctggggctttatccctcttgtgatttacctgggatttaagaggggtgcagatcccggaatgcctgaaccaactgttttgagcctactttggggatag

SEQ ID NO: 22 - DNA, 35 bp, Artificial Sequence
  Rp53 primer
aaaaaagaattcatggtctgagtcaggcccttctg

SEQ ID NO: 23 - DNA, 1266 bp, Artificial Sequence
  nucleotides sequence coding p53-myc/His
atggaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacctcgagtctagagggcccttcgaacaaaaactcatctcagaagaggatctgaatatgcataccggtcatcatcaccatcaccattga

SEQ ID NO: 24 - DNA, 51 bp, Artificial Sequence
  T2GZMB primer
aaaaaaccgcggtggtatcatcgggggacatgaggcacatgaggccaagcc

SEQ ID NO: 25 - DNA, 38 bp, Artificial Sequence
  XGZMB(noT) primer
aaaaaactcgaggtagcgtttcatggttttctttatcc

SEQ ID NO: 26 - DNA, 691 bp, Artificial Sequence
  nucleotides sequence coding GranzymeB
ccgcggtggtatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctac

SEQ ID NO: 27 - DNA, 1065 bp, Artificial Sequence
  nucleotides sequence coding TOM70-(GGGGS)3-UB-Granzyme B
atgaaaagttttataactcggaataaaactgcaattttcgcaactgttgctgctacgggaaccgctattggtgcatactactattatggaggtggcggaagcgggggtggaggatccgggggcggcggaagccaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagcaccaccaccaccaccactag

SEQ ID NO: 28 - DNA, 1083 bp, Artificial Sequence
  nucleotides sequence coding UB-Granzyme B-TOM7
atgggcagcagccatcatcatcatcatcacagcagcggcctggtgccgcgcggcagccatatgcaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagtttgccattcgctggggctttatccctcttgtgatttacctgggatttaagaggggtgcagatcccggaatgcctgaaccaactgttttgagcctactttggggatag

SEQ ID NO: 29 - DNA, 39 bp, Artificial Sequence
  T2RKIP primer
aaaaaaccgcggtggtccggtggacctcagcaagtggtc

SEQ ID NO: 30 - DNA, 41 bp, Artificial Sequence
  XRKIP(noT) primer
aaaaaactcgagcttcccagacagctgctcgtacagtttgg

SEQ ID NO: 31 - DNA, 568 bp, Artificial Sequence
  nucleotides sequence coding RKIP
ccgcggtggtccggtggacctcagcaagtggtccgggcccttgagcctgcaagaagtggacgagcagccgcagcacccactgcatgtcacctacgccggggcggcggtggacgagctgggcaaagtgctgacgcccacccaggttaagaatagacccaccagcatttcgtgggatggtcttgattcagggaagctctacaccttggtcctgacagacccggatgctcccagcaggaaggatcccaaatacagagaatggcatcatttcctggtggtcaacatgaagggcaatgacatcagcagtggcacagtcctctccgattatgtgggctcggggcctcccaagggcacaggcctccaccgctatgtctggctggtttacgagcaggacaggccgctaaagtgtgacgagcccatcctcagcaaccgatctggagaccaccgtggcaaattcaaggtggcgtccttccgtaaaaagtatgagctcagggccccggtggctggcacgtgttaccaggccgagtgggatgactatgtgcccaaactgtacgagcagctgtctgggaag

SEQ ID NO: 32 - DNA, 942 bp, Artificial Sequence
  nucleotides sequence coding TOM70-(GGGGS)3-UB-RKIP
atgaaaagttttataactcggaataaaactgcaattttcgcaactgttgctgctacgggaaccgctattggtgcatactactattatggaggtggcggaagcgggggtggaggatccgggggcggcggaagccaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtccggtggacctcagcaagtggtccgggcccttgagcctgcaagaagtggacgagcagccgcagcacccactgcatgtcacctacgccggggcggcggtggacgagctgggcaaagtgctgacgcccacccaggttaagaatagacccaccagcatttcgtgggatggtcttgattcagggaagctctacaccttggtcctgacagacccggatgctcccagcaggaaggatcccaaatacagagaatggcatcatttcctggtggtcaacatgaagggcaatgacatcagcagtggcacagtcctctccgattatgtgggctcggggcctcccaagggcacaggcctccaccgctatgtctggctggtttacgagcaggacaggccgctaaagtgtgacgagcccatcctcagcaaccgatctggagaccaccgtggcaaattcaaggtggcgtccttccgtaaaaagtatgagctcagggccccggtggctggcacgtgttaccaggccgagtgggatgactatgtgcccaaactgtacgagcagctgtctgggaagctcgagcaccaccaccaccaccactag

SEQ ID NO: 33 - DNA, 42 bp, Artificial Sequence
  T2PTEN primer
aaaaaaccgcggtggtacagccatcatcaaagagatcgttag

SEQ ID NO: 34 - DNA, 36 bp, Artificial Sequence
  XPTEN(noT) primer
aaaaaactcgaggacttttgtaatttgtgtatgctg

SEQ ID NO: 35 - DNA, 1216 bp, Artificial Sequence
  nucleotides sequence coding PTEN
ccgcggtggtacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtc

SEQ ID NO: 36 - DNA, 1590 bp, Artificial Sequence
  nucleotides sequence coding TOM70-(GGGGS)3-UB- PTEN
atgaaaagttttataactcggaataaaactgcaattttcgcaactgttgctgctacgggaaccgctattggtgcatactactattatggaggtggcggaagcgggggtggaggatccgggggcggcggaagccaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtcctcgagcaccaccaccaccaccactag

SEQ ID NO: 37 - DNA, 739 bp, Artificial Sequence
  nucleotides sequence coding scFvHER2
ccgcggtggtgaagtgcaacttgttgagagtggcggaggactggtccaaccgggcggttcacttaggctctcatgtgcagcttcagggttcacgttcacggactatacaatggactgggtgaggcaagcccctggtaagggactggagtgggttgctgacgttaaccctaattccggtgggtccatctacaaccagcgattcaagggacgatttactctttcagtcgacagaagcaaaaacaccctctacctccagatgaactccttgcgggcagaggatacagcggtctactattgcgcgagaaacttgggaccaagcttctacttcgactactgggggcaaggaacgcttgttacggtttcaagcggaggtggaggaagtggaggtggcggttccggcggtggcggttcagatatacagatgacccaatcacccagttctcttagcgcgtctgtaggcgacagggtaaccataacctgcaaggcgtcccaggacgtgtcaattggagttgcctggtatcagcaaaaacctgggaaagctccgaagctcctgatttacagcgcatcttaccgatatactggtgtcccttcaaggttcagtggcagtggatctgggacagactttacgcttactatcagcagtctgcaacctgaggatttcgcgacctactactgtcagcagtattacatctatccgtacacgttcggtcaaggtacaaaggtagaaataaaacgcact

SEQ ID NO: 38 - DNA, 1125 bp, Artificial Sequence
  nucleotides sequence coding UB-scFvHER2-TOM7
atgggcagcagccatcatcatcatcatcacagcagcggcctggtgccgcgcggcagccatatgcaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtgaagtgcaacttgttgagagtggcggaggactggtccaaccgggcggttcacttaggctctcatgtgcagcttcagggttcacgttcacggactatacaatggactgggtgaggcaagcccctggtaagggactggagtgggttgctgacgttaaccctaattccggtgggtccatctacaaccagcgattcaagggacgatttactctttcagtcgacagaagcaaaaacaccctctacctccagatgaactccttgcgggcagaggatacagcggtctactattgcgcgagaaacttgggaccaagcttctacttcgactactgggggcaaggaacgcttgttacggtttcaagcggaggtggaggaagtggaggtggcggttccggcggtggcggttcagatatacagatgacccaatcacccagttctcttagcgcgtctgtaggcgacagggtaaccataacctgcaaggcgtcccaggacgtgtcaattggagttgcctggtatcagcaaaaacctgggaaagctccgaagctcctgatttacagcgcatcttaccgatatactggtgtcccttcaaggttcagtggcagtggatctgggacagactttacgcttactatcagcagtctgcaacctgaggatttcgcgacctactactgtcagcagtattacatctatccgtacacgttcggtcaaggtacaaaggtagaaataaaacgcacttttgccattcgctggggctttatccctcttgtgatttacctgggatttaagaggggtgcagatcccggaatgcctgaaccaactgttttgagcctactttggggatag

SEQ ID NO: 39 - DNA, 38 bp, Artificial Sequence
  RscFvHER2 primer
aaaaaagaattcatggaagtgcaacttgttgagagtgg

SEQ ID NO: 40 - DNA, 35 bp, Artificial Sequence
  XTOM7(noT) primer
aaaaaactcgagtccccaaagtaggctcaaaacag

SEQ ID NO: 41 - DNA, 924 bp, Artificial Sequence
  nucleotides sequence coding scFvHER2-TOM7-myc/ His
atggaagtgcaacttgttgagagtggcggaggactggtccaaccgggcggttcacttaggctctcatgtgcagcttcagggttcacgttcacggactatacaatggactgggtgaggcaagcccctggtaagggactggagtgggttgctgacgttaaccctaattccggtgggtccatctacaaccagcgattcaagggacgatttactctttcagtcgacagaagcaaaaacaccctctacctccagatgaactccttgcgggcagaggatacagcggtctactattgcgcgagaaacttgggaccaagcttctacttcgactactgggggcaaggaacgcttgttacggtttcaagcggaggtggaggaagtggaggtggcggttccggcggtggcggttcagatatacagatgacccaatcacccagttctcttagcgcgtctgtaggcgacagggtaaccataacctgcaaggcgtcccaggacgtgtcaattggagttgcctggtatcagcaaaaacctgggaaagctccgaagctcctgatttacagcgcatcttaccgatatactggtgtcccttcaaggttcagtggcagtggatctgggacagactttacgcttactatcagcagtctgcaacctgaggatttcgcgacctactactgtcagcagtattacatctatccgtacacgttcggtcaaggtacaaaggtagaaataaaacgcacttttgccattcgctggggctttatccctcttgtgatttacctgggatttaagaggggtgcagatcccggaatgcctgaaccaactgttttgagcctactttggggactcgagtctagagggcccttcgaacaaaaactcatctcagaagaggatctgaatatgcataccggtcatcatcaccatcaccattga

SEQ ID NO: 42 - DNA, 760 bp, Artificial Sequence
  nucleotides sequence coding scFvMEL
ccgcggtggtacggacattgtgatgacccagtctcaaaaattcatgtccacatcagtaggagacagggtcagcgtcacctgcaaggccagtcagaatgtggatactaatgtagcctggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggaggcaccaagctggagatcaaaggctccaccagcggcagcggtaagccaggctccggcgaaggcagcaccaaaggcgaagtgaaggttgaggagtctggaggaggcttggtgcaacctggaggatccatgaaactctcctgtgttgtctctggattcactttcggtaattactggatgaactgggtccgccagtctccagagaaggggcttgagtggattgcagaaattagattgaaatccaataattttgcaagatattatgcggagtctgtgaaagggaggttcaccatctcaagagatgattccaaaagtagtgtctacctgcaaatgatcaacctaagagctgaagatactggcatttattactgtaccagttatggtaactacgttgggcactattttgaccactggggccaaggcaccactctcaccgtctccctttgggga

SEQ ID NO: 43 - DNA, 1137 bp, Artificial Sequence
  nucleotides sequence coding UB-scFvMEL-TOM7
atgggcagcagccatcatcatcatcatcacagcagcggcctggtgccgcgcggcagccatatgcaaatcttcgtcaaaactctaacagggaagactataaccctagaggttgaaccatccgacactattgaaaacgtcaaagctaaaattcaagataaagaaggtatccctccggatcagcagagattgatttttgctggtaagcaactagaagatggtagaaccttgtctgactacaacatccaaaaggaatctactcttcacttggtgttgagactccgcggtggtacggacattgtgatgacccagtctcaaaaattcatgtccacatcagtaggagacagggtcagcgtcacctgcaaggccagtcagaatgtggatactaatgtagcctggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggaggcaccaagctggagatcaaaggctccaccagcggcagcggtaagccaggctccggcgaaggcagcaccaaaggcgaagtgaaggttgaggagtctggaggaggcttggtgcaacctggaggatccatgaaactctcctgtgttgtctctggattcactttcggtaattactggatgaactgggtccgccagtctccagagaaggggcttgagtggattgcagaaattagattgaaatccaataattttgcaagatattatgcggagtctgtgaaagggaggttcaccatctcaagagatgattccaaaagtagtgtctacctgcaaatgatcaacctaagagctgaagatactggcatttattactgtaccagttatggtaactacgttgggcactattttgaccactggggccaaggcaccactctcaccgtctcctcatttgccattcgctggggctttatccctcttgtgatttacctgggatttaagaggggtgcagatcccggaatgcctgaaccaactgttttgagcctactttgggga

SEQ ID NO: 44 - DNA, 36 bp, Artificial Sequence
  RscFvMEL primer
aaaaaagaattcatgaaaacaagtaacccaggagtg

SEQ ID NO: 45 - DNA, 939 bp, Artificial Sequence
  nucleotides sequence coding scFvMEL-TOM7-myc/ His
atgacggacattgtgatgacccagtctcaaaaattcatgtccacatcagtaggagacagggtcagcgtcacctgcaaggccagtcagaatgtggatactaatgtagcctggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggaggcaccaagctggagatcaaaggctccaccagcggcagcggtaagccaggctccggcgaaggcagcaccaaaggcgaagtgaaggttgaggagtctggaggaggcttggtgcaacctggaggatccatgaaactctcctgtgttgtctctggattcactttcggtaattactggatgaactgggtccgccagtctccagagaaggggcttgagtggattgcagaaattagattgaaatccaataattttgcaagatattatgcggagtctgtgaaagggaggttcaccatctcaagagatgattccaaaagtagtgtctacctgcaaatgatcaacctaagagctgaagatactggcatttattactgtaccagttatggtaactacgttgggcactattttgaccactggggccaaggcaccactctcaccgtctcctcatttgccattcgctggggctttatccctcttgtgatttacctgggatttaagaggggtgcagatcccggaatgcctgaaccaactgttttgagcctactttggggactcgagtctagagggcccttcgaacaaaaactcatctcagaagaggatctgaatatgcataccggtcatcatcaccatcaccattga

SEQ ID NO: 46 - DNA, 750 bp, Artificial Sequence
  nucleotides sequence coding scFvPD-L1
atggacatcgtgatgagccagtctcccagcagcctggctgtgtctgctggggagaaggtgaccatgtcctgcaagagctcccagtccctgctgaacagccgcaccaggaagaactacctggcctggtaccagcagaagccaggccagagccccaagctcctcatctactgggccagcacccgggagagcggggtgcctgaccgcttcactggaagtggcagcggcacagacttcaccctgaccatcagctctgtgcaggccgaggacctggcagtgtactactgccagcaaagctatgatgtggtgacatttggagctggcaccaagctggagctgaagggaggtggcggaagcgggggtggaggatccgggggcggcggaagccaggtccaggtgcagcagagcggggctgagctggccgagcccggggcctctgtgaagatgagctgcaaggcttctggctacatcttcaccagctactggatgcactggctcaagcagaggcctgggcaggggctggagtggatcggctatatcaaccccagcagtgactacaatgaatattctgagaagttcatggacaaagccaccctgactgctgacaaggccagcaccaccgcctacatgcagctgatcagcctgacctcagaggacagcgctgtgtactactgtgcccggagcggctggctggtgcacggggactattattttgattattggggccagggcaccacactgacagtgagcagc

SEQ ID NO: 47 - DNA, 948 bp, Artificial Sequence
  nucleotides sequence coding scFvPD-L1-TOM7-myc/ His
atggacatcgtgatgagccagtctcccagcagcctggctgtgtctgctggggagaaggtgaccatgtcctgcaagagctcccagtccctgctgaacagccgcaccaggaagaactacctggcctggtaccagcagaagccaggccagagccccaagctcctcatctactgggccagcacccgggagagcggggtgcctgaccgcttcactggaagtggcagcggcacagacttcaccctgaccatcagctctgtgcaggccgaggacctggcagtgtactactgccagcaaagctatgatgtggtgacatttggagctggcaccaagctggagctgaagggaggtggcggaagcgggggtggaggatccgggggcggcggaagccaggtccaggtgcagcagagcggggctgagctggccgagcccggggcctctgtgaagatgagctgcaaggcttctggctacatcttcaccagctactggatgcactggctcaagcagaggcctgggcaggggctggagtggatcggctatatcaaccccagcagtgactacaatgaatattctgagaagttcatggacaaagccaccctgactgctgacaaggccagcaccaccgcctacatgcagctgatcagcctgacctcagaggacagcgctgtgtactactgtgcccggagcggctggctggtgcacggggactattattttgattattggggccagggcaccacactgacagtgagcagcctcgagtttgccattcgctggggctttatccctcttgtgatttacctgggatttaagaggggtgcagatcccggaatgcctgaaccaactgttttgagcctactttggggactcgagtctagagggcccttcgaacaaaaactcatctcagaagaggatctgaatatgcataccggtcatcatcaccatcaccattga

SEQ ID NO: 48 - protein, 395 aa, Artificial Sequence
  amino acid sequence of p53
RGGEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 49 - protein, 488 aa, Artificial Sequence
  amino acid sequence of UB-p53
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 50 - protein, 504 aa, Artificial Sequence
  amino acid sequence of TOM70-UB-p53
MKSFITRNKTAIFATVAATGTAIGAYYYYQIFVKTLTGKTITLEVEPSDTIENVKAKIQDKEGIPPDQQRLIFAGKQLEDGRTLSDYNIQKESTLHLVLRLRGGEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLEHHHHHH

SEQ ID NO: 51 - protein, 519 aa, Artificial Sequence
  amino acid sequence of TOM70-(GGGGS)3-UB-p53
MKSFITRNKTAIFATVAATGTAIGAYYYYGGGGSGGGGSGGGGSQIFVKTLTGKTITLEVEPSDTIENVKAKIQDKEGIPPDQQRLIFAGKQLEDGRTLSDYNIQKESTLHLVLRLRGGEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLEHHHHHH

SEQ ID NO: 52 - protein, 444 aa, Artificial Sequence
  amino acid sequence of TOM70-(GGGGS)3-p53
MKSFITRNKTAIFATVAATGTAIGAYYYYGGGGSGGGGSGGGGSEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLEHHHHHH

SEQ ID NO: 53 - protein, 525 aa, Artificial Sequence
  amino acid sequence of UB-p53-TOM7
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVHVCACPGRDRRTEEENLRKKGEPHHELPPGSTKRALSNNTSSSPQPKKKPLDGEYFTLQIRGRERFEMFRELNEALELKDAQAGKEPGGSRAHSSHLKSKKGQSTSRHKKLMFKTEGPDSDLEFAIRWGFIPLVIYLGFKRGADPGMPEPTVLSLLWG

SEQ ID NO: 54 - protein, 421 aa, Artificial Sequence
  amino acid sequence of p53-myc/His
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLESRGPFEQKLISEEDLNMHTGHHHHHH

SEQ ID NO: 55 - protein, 230 aa, Artificial Sequence
  amino acid sequence of GranzymeB
RGGIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 56 - protein, 354 aa, Artificial Sequence
  amino acid sequence of TOM70-(GGGGS)3-UB- Granzyme B
MKSFITRNKTAIFATVAATGTAIGAYYYYGGGGSGGGGSGGGGSQIFVKTLTGKTITLEVEPSDTIENVKAKIQDKEGIPPDQQRLIFAGKQLEDGRTLSDYNIQKESTLHLVLRLRGGIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYLEHHHHHH

SEQ ID NO: 57 - protein, 360 aa, Artificial Sequence
  amino acid sequence of UB-Granzyme B-TOM7
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYLEFAIRWGFIPLVIYLGFKRGADPGMPEPTVLSLLWG

SEQ ID NO: 58 - protein, 189 aa, Artificial Sequence
  amino acid sequence of RKIP
RGGPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 59 - protein, 313 aa, Artificial Sequence
  amino acid sequence of TOM70-(GGGGS)3-UB-RKIP
MKSFITRNKTAIFATVAATGTAIGAYYYYGGGGSGGGGSGGGGSQIFVKTLTGKTITLEVEPSDTIENVKAKIQDKEGIPPDQQRLIFAGKQLEDGRTLSDYNIQKESTLHLVLRLRGGPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGKLEHHHHHH

SEQ ID NO: 60 - protein, 405 aa, Artificial Sequence
  amino acid sequence of PTEN
RGGTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV

SEQ ID NO: 61 - protein, 529 aa, Artificial Sequence
  amino acid sequence of TOM70-(GGGGS)3-UB-PTEN
MKSFITRNKTAIFATVAATGTAIGAYYYYGGGGSGGGGSGGGGSQIFVKTLTGKTITLEVEPSDTIENVKAKIQDKEGIPPDQQRLIFAGKQLEDGRTLSDYNIQKESTLHLVLRLRGGTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKVLEHHHHHH

SEQ ID NO: 62 - protein, 246 aa, Artificial Sequence
  amino acid sequence of scFvHER2
RGGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRT

SEQ ID NO: 63 - protein, 374 aa, Artificial Sequence
  amino acid sequence of UB-scFvHER2-TOM7
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTFAIRWGFIPLVIYLGFKRGADPGMPEPTVLSLLWG

SEQ ID NO: 64 - protein, 307 aa, Artificial Sequence
  amino acid sequence of scFvHER2-TOM7-myc/His
MEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSGGGGSGGGGSGGGGSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTFAIRWGFIPLVIYLGFKRGADPGMPEPTVLSLLWGLESRGPFEQKLISEEDLNMHTGHHHHHH

SEQ ID NO: 65 - protein, 251 aa, Artificial Sequence
  amino acid sequence of scFvMEL
RGGTDIVMTQSQKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKGSTSGSGKPGSGEGSTKGEVKVEESGGGLVQPGGSMKLSCVVSGFTFGNYWMNWVRQSPEKGLEWIAEIRLKSNNFARYYAESVKGRFTISRDDSKSSVYLQMINLRAEDTGIYYCTSYGNYVGHYFDHWGQGTTLTVSS

SEQ ID NO: 66 - protein, 378 aa, Artificial Sequence
  amino acid sequence of UB-scFvMEL-TOM7
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGTDIMTQSQKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKGSTSGSGKPGSGEGSTKGEVKVEESGGGLVQPGGSMKLSCVVSGFTFGNYWMNWVRQSPEKGLEWIAEIRLKSNNFARYYAESVKGRFTISRDDSKSSVYLQMINLRAEDTGIYYCTSYGNYVGHYFDHWGQGTTLTVSSFAIRWGFIPLVIYLGFKRGADPGMPEPTVLSLLWG

SEQ ID NO: 67 - protein, 312 aa, Artificial Sequence
  amino acid sequence of scFvMEL-TOM7-myc/His
MTDIVMTQSQKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKGSTSGSGKPGSGEGSTKGEVKVEESGGGLVQPGGSMKLSCVVSGFTFGNYWMNWVRQSPEKGLEWIAEIRLKSNNFARYYAESVKGRFTISRDDSKSSVYLQMINLRAEDTGIYYCTSYGNYVGHYFDHWGQGTTLTVSSFAIRWGFIPLVIYLGFKRGADPGMPEPTVLSLLWGLESRGPFEQKLISEEDLNMHTGHHHHHH

SEQ ID NO: 68 - protein, 250 aa, Artificial Sequence
  amino acid sequence of scFvPD-L1
MDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCQQSYDVVTFGAGTKLELKGGGGSGGGGSGGGGSQVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSS

SEQ ID NO: 69 - protein, 315 aa, Artificial Sequence
  amino acid sequence of scFvPD-L1-TOM7-myc/His
MDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCQQSYDVVTFGAGTKLELKGGGGSGGGGSGGGGSQVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSSLEFAIRWGFIPLVIYLGFKRGADPGMPEPTVLSLLWGLESRGPFEQKLISEEDLNMHTGHHHHHH

SEQ ID NO: 70 - DNA, 15 bp, Artificial Sequence
  linker
ggggsggggsggggs

SEQ ID NO: 71 - protein, 75 aa, Artificial Sequence
  amino acid sequence of ubiquitin
QLFVKTLTGKTVTLEVESSDTIDNVKSKIQDKEGIPPDQQRLIFAGKQLEDGRTLSDYNIQKESTLHLVLRLRGG

SEQ ID NO: 72 - DNA, 225 bp, Artificial Sequence
  nucleotides sequence coding ubiquitin
caacttttcgtcaaaactctaacagggaagactgtaaccctagaggttgaatcttccgacactattgacaacgtcaaaagtaaaattcaagataaagaaggtatccctccggatcagcagagattgatttttgctggtaagcaactagaagatggtagaaccttgtctgactacaacatccaaaaggaatctactcttcacttggtgttgagactccgcggtggt

SEQ ID NO: 73 - protein, 400 aa, Artificial Sequence
  amino acid sequence of p53
EEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLEHHHHHH

SEQ ID NO: 74 - DNA, 1203 bp, Artificial Sequence
  nucleotides sequence coding p53
gaggagccgcagtcagatcctagcgtcgagccccctctgagtcaggaaacattttcagacctgtggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacctcgagcaccaccaccaccaccactag

SEQ ID NO: 75 - protein, 29 aa, Artificial Sequence
  TOM70 of S. cerevisiae
MKSFITRNKTAIFATVAATGTAIGAYYYY

SEQ ID NO: 76 - protein, 60 aa, Artificial Sequence
  TOM70 of Homo sapiens
MAASKPVEAAVVAAAVPSSGSGVGGGGTAGPGTGGLPRWQLALAVGAPLLLGAGAIYLWS

SEQ ID NO: 77 - protein, 29 aa, Artificial Sequence
  TOM20 of S. cerevisiae
MSQSNPILRGLAITTAIAALSATGYAIYF

SEQ ID NO: 78 - protein, 24 aa, Artificial Sequence
  TOM20 of Homo sapiens
MVGRNSAIAAGVCGALFIGYCIYF

SEQ ID NO: 79 - protein, 22 aa, Artificial Sequence
  OM45 of S. cerevisiae
MSSRIIVGSAALAAAITASIMV

SEQ ID NO: 80 - protein, 23 aa, Artificial Sequence
  TOM5 of S. cerevisiae
AAYVAAFLWVSPMIWHLVKKQWK

SEQ ID NO: 81 - protein, 24 aa, Artificial Sequence
  TOM5 of Homo sapiens
IRNFLIYVALLRVTPFILKKLDSI

SEQ ID NO: 82 - protein, 41 aa, Artificial Sequence
  TOM7 of S. cerevisiae
ILTLTHNVAHYGWIPFVLYLGWAHTSNRPNFLNLLSPLPSV

SEQ ID NO: 83 - protein, 35 aa, Artificial Sequence
  TOM7 of Homo sapiens
FAIRWGFIPLVIYLGFKRGADPGMPEPTVLSLLWG

SEQ ID NO: 84 - protein, 55 aa, Artificial Sequence
  TOM22 of S. cerevisiae
LAWTLTTTALLLGVPLSLSILAEQQLIEMEKTFDLQSDANNILAQGEKDAAATAN

SEQ ID NO: 85 - protein, 60 aa, Artificial Sequence
  TOM22 of Homo sapiens
AALWIGTTSFMILVLPVVFETEKLQMEQQQQLQQRQILLGPNTGLSGGMPGALPSLPGKI

SEQ ID NO: 86 - protein, 25 aa, Artificial Sequence
  Fis1 of S. cerevisiae
GVVVAGGVLAGAVAVASFFLRNKRR

SEQ ID NO: 87 - protein, 31 aa, Artificial Sequence
  Fis1 of Homo sapiens
GLVGMAIVGGMALGVAGLAGLIGLAVSKSKS

SEQ ID NO: 88 - protein, 25 aa, Artificial Sequence
  Bcl-2 alpha of Homo sapiens
LSLKTLLSLALVGACITLGAYLGHK

SEQ ID NO: 89 - protein, 19 aa, Artificial Sequence
  VAMP1 of S. cerevisiae
MIMLGAICAIIVVVIVRRD

SEQ ID NO: 90 - protein, 22 aa, Artificial Sequence
  VAMP1 of Homo sapiens
MMIMLGAICAIIVVVIVIYFFT

SEQ ID NO: 91 - DNA, 60 bp, Artificial Sequence
  P53-promter-S
gggcatgctcgggcatgcccgggcatgctcgggcatgcccgggcatgctcgggcatgccc

SEQ ID NO: 92 - DNA, 60 bp, Artificial Sequence
  P53-promter-AS
gggcatgcccgagcatgcccgggcatgcccgagcatgcccgggcatgcccgagcatgccc

SEQ ID NO: 93 - protein, 4 aa, Artificial Sequence
  Synthetic fragment of ubiquitin
LRGG

SEQ ID NO: 94 - protein, 5 aa, Artificial Sequence
  Synthetic fragment of ubiquitin
RLRGG

SEQ ID NO: 95 - protein, 6 aa, Artificial Sequence
  Synthetic fragment of ubiquitin
LRLRGG